(19) United States
(12) Patent Application Publication
(10) Pub. No.: US 2025/0144153 A1
(43) Pub. Date: May 8, 2025

(54) METHODS AND COMPOSITIONS FOR TREATING DRY EYE, TEAR HYPEROSMOLARITY-INDUCED PATHOLOGICAL CHANGES, AND OTHER OCULAR CONDITIONS

(71) Applicant: MAM Holdings of West Florida, L.L.C., Palm Harbor, FL (US)

(72) Inventor: Carl Randall Harrell, Tarpon Springs, FL (US)

(21) Appl. No.: 18/770,542

(22) Filed: Jul. 11, 2024

Related U.S. Application Data
(60) Provisional application No. 63/526,628, filed on Jul. 13, 2023.

(51) Int. Cl. A61K 35/50; A61K 9/00; A61K 9/08; A61K 35/28; A61K 38/17; A61P 27/04; A61P 27/06; A61P 37/06

(52) U.S. Cl. CPC A61K 35/50; A61K 9/0048; A61K 9/08; A61K 35/28; A61K 38/1709; A61P 27/04; A61P 27/06; A61P 37/06

(57) ABSTRACT

The present disclosure is directed to compositions, formulations, and methods for treating diseases of the eye and other disorders. Specifically, d-MAPPS compositions, which may include d-MAPPS solutions (e.g., in liquid form and/or administered as eye drops), in addition to macromolecular proteins, growth factors, mesenchymal stem cells (MSC), can be used for topical application to the eye and treatment of, for instance, tear hyperosmolarity (TH), dry eye, dry eye disease, dry eye discomfort, tear hyperosmolarity, and tear hyperosmolarity-induced pathological changes in the eyes of patients. The d-MAPPS solutions can contain mesenchymal stem cells (MSC), MSC-derived exosomes (MSC-Exos), one or more MSC-sourced growth factors, and/or immunoregulatory proteins. In some embodiments, the d-MAPPS solutions can include a sterile de-cellularized human amniotic fluid (D-HAF). In embodiments, the d-MAPPS solutions are amenable to long-term storage without the loss of biological potency.

### CROSS-REFERENCE TO RELATED APPLICATION

This application claims priority to U.S. Provisional Application No. 63/295,423, filed Jul. 13, 2023, which is hereby incorporated by reference in its entirety.

### STATEMENT REGARDING FEDERALLY SPONSORED RESEARCH

This invention was made without government support or grants awarded by the National Institutes of Health. The government has no rights in the invention.

### FIELD OF THE DISCLOSURE

The present disclosure relates to methods and composition for treating ocular conditions, including, but not limited to, tear hyperosmolarity and dry eye disease. Various embodiments of the disclosure relate to methods and compositions for the prevention, management, and treatment of various ophthalmic diseases, ocular injuries, and other disorders. In certain embodiments, therapeutic compositions may include, for example, macromolecular proteins, growth factors, mesenchymal stem cells (MSC), and various immunoregulatory biomolecules.

### BACKGROUND

A primary function of the mammalian cornea and its surrounding structures is to moisten the eye. When the lubricated state of the eye is impaired, the subject may suffer from various ocular conditions, including, but not limited to, tear hyperosmolarity (THO) and/or dry eye disease (referred to interchangeably herein as DE or DED). Such conditions are often common, multifactorial diseases of the lacrimal system and ocular surface, and are characterized by a deficiency in quality and/or quantity of the tear fluid. Dehydration of moisture from the eye of the subject gives rise to various discomforts related to ocular dryness as well as burning and scratching sensations.

An even more serious consequence of a dry eye condition is the loss of visual acuity, which if not corrected, may result in permanent damage. In fact, dry eye disease may act to degrade the exposed ocular surface and may cause a complete breakdown of corneal tissues. In an extreme case, this may necessitate a corneal transplant. In advanced cases, decreased tear secretion or altered tear composition leads to tear film instability/imbalance which, in DE patients, can result in the abnormally rapid breakup of the tear film. The lives of people with DE are negatively impacted due to consistent pain, redness and/or dryness of the eyes. Typically, artificial tears and over-the counter eye drops are used by patients to sooth eye irritation and to lubricate the eyes. The majority of these treatment options provide minimal relief for limited duration and require several daily reapplications.

These treatment options provide varying degrees of relief with limited capacity to modify the underlying disease state. Human amniotic membrane (HAM) has been used efficaciously to treat specific eye surface injuries and maladies. However, the use of HAM often involves the skills of a physician and additional expense to patients. Additionally, these procedures usually impose severe vision impairment during treatment as the amniotic membrane is non-transparent. Ultimately, the benefits of the procedure last only as long as the membrane is in place, so the procedure is not particularly useful for chronic conditions such as dry eye, dry eye discomfort, and tear hyperosmolarity, which is an important step in the development, progression, and aggravation of dry eye discomfort.

Mesenchymal stem cells (“MSC” or “MSCs”) are self-renewable, multipotent stem cells that regulate innate and/or adaptive immune responses in various human tissues. For instance, MSCs play a role in responding to tissue injury and reducing inflammation. Moreover, due to their immunosuppressive properties, MSCs have therapeutic potential in alleviating various diseases (e.g., ophthalmic diseases, ocular diseases, autoimmune diseases, and specific cancers). MSCs may originate from different sources (e.g., bone marrow, amniotic fluid, placental tissue, etc.) and contain a variety of biological compounds (e.g., carbohydrates, proteins and peptides, lipids, lactate, pyruvate, electrolytes, enzymes, hormones, and various growth factors).

Tear hyperosmolarity, dysfunction of ion channel proteins and eye inflammation are mainly responsible for the development and progression of DED. Alterations in the structure and/or function of ion channel receptors and consequent hyperosmolarity of the tears represent the initial step in development and progression of DED. Hyperosmolarity triggers the activation of the ion channel-dependent signaling pathways in corneal epithelial cells and eye-infiltrated immune cells, leading to the activation of transcriptional factors which enhance expression of genes that regulate production of inflammatory cytokines, resulting in the generation of potent inflammatory response in the eyes of DED patients. Long-lasting and un-treated, detrimental immune response, in turn, causes modifications in the structure and/or function of ion channel proteins which leads to the tear hyperosmolarity and generates a vicious circle in the eyes of DED patients, aggravating DED-related signs and symptoms. Accordingly, suppression immune cell-driven eye inflammation and alleviation of tear hyperosmolarity through the modulation of ion channels in the eyes of DED patients hold promise for developing new therapeutic strategies for managing DED. Herewith, we summarized current knowledge about the molecular mechanisms which are responsible for the inflammation-induced modification of ion channels leading to the generation of tear hyperosmolarity that elicit immune cell dysfunction in the eyes of DED patients. We also emphasized therapeutic potential of newly designed immunomodulatory and hypo-osmotic solution, d-MAPPS®, which can stabilize the tear film, enhance natural cytokine communication and suppress detrimental immune response, opening new avenues for addressing tear hyperosmolarity-dependent immune cell-driven inflammation in the eyes of DED patients, potentially revolutionizing DED management.

The multifactorial nature of DED encompasses a number of interconnected underlying pathologies, including loss of homeostasis, instability and hyperosmolarity of the tears, and the induction and propagation of detrimental inflammatory responses in the eyes, which finally results in the development of neurosensory dysfunction and visual disruption. Dryness, grittiness, scratchiness, discomfort, inflammation, burning, watering, ocular fatigue, pain, and decreased functional visual acuity are common symptoms of DED. Eye dysfunction drastically attenuates patients' quality of life. Accordingly, a better understanding of the pathogenic processes that regulate the development and progression of DED is crucially important for the establishment of new and more effective DED-related treatment approaches, which would significantly improve the quality of life of DED patients. Since the process of osmoregulation, which guards the ocular surface epithelia and maintains normal vision, is affected when the osmolarity of the tears is greater than that of the epithelial cells, tear hyperosmolarity (THO) is considered an initial, important step in the development, progression, and aggravation of DED. In order to delineate the role of THO in the pathogenesis of DED, in this review article, we summarize current knowledge related to the molecular mechanisms responsible for the development of THO-induced pathological changes in the eyes of DED patients, and we briefly discuss the therapeutic potential of hypo-osmotic eye drops in DED treatment.

Dry eye disease (DED) is a common eye condition affecting over half the world's population. This multifactorial disorder, which affects both the lacrimal system and the ocular surface, is characterized by a deficiency in the quality and/or quantity of the tear fluid. DED has two primary subtypes: evaporative dry eye (EDE), caused by excessive evaporation of tear fluid; and aqueous tear-deficient dry eye (ADDE), characterized by the lacrimal glands' inefficiency or failure to produce tears. In clinical settings, DED is often seen as a “combination” or “hybrid” form of these two types, where each type gains certain clinical characteristics from the other, initiating and worsening its pathology.

Many factors, including aging, eye injury, meibomian gland dysfunction (MGD), surgical interventions in the eye, poor nutrition, environmental allergens and chemical hazards, can cause dysfunction of ion channel proteins and may generate detrimental immune response in the eye, resulting in the development and progression of DED. Tear hyperosmolarity, (caused by alterations in ion channels) and ocular inflammation (caused by enhanced activation of immune cells and massive production of inflammatory cytokines) create a vicious circle, leading to the aggravation of all main DED-related signs and symptoms (ocular discomfort, pain, and vision problems).

Accordingly, many DED-related experimental and clinical studies focused on the examination of ion channel-dependent effects on the development of DED-associated pathology. Additionally, delineation of signaling pathways which are elicited in eye-infiltrated immune cells during DED progression, paved the way for the design of new immunoregulatory therapeutic agents which were able to suppress detrimental immunity in the eye, attenuating DED-related signs and symptoms. Therefore, the disclosure herein emphasizes therapeutic potential of newly designed immunomodulatory and hypo-osmotic solution, d-MAPPS®, which can stabilize the tear film, enhance natural cytokine communication and suppress detrimental immune response, opening new avenues for addressing tear hyperosmolarity-dependent immune cell-driven inflammation in the eyes of DED patients, potentially revolutionizing DED management. In view of the foregoing, there is a significant need for compositions and formulations that can be used for the management of various eye diseases, injuries and disorders, and that are affordable, readily accessible, and easy to use for both clinician and patient. In particular, there is a need for compositions and/or formulations that provide for the clinical use of d-MAPPS®, MSCs and/or MSC-derived products (e.g., exosomes, growth factors, immunoregulatory proteins, etc.) in preventing, managing, and/or treating various eye diseases

### SUMMARY

It is to be understood that both the following summary and the detailed description are exemplary and explanatory and are intended to provide further explanation of the invention as claimed. Neither the summary nor the description that follows is intended to define or limit the scope of the invention to the particular features mentioned in the summary or in the description.

In certain embodiments, the disclosed embodiments may include one or more of the features described herein.

Embodiments of the present disclosure are directed towards compositions, formulations, and methods for using one or more types of mesenchymal stem cells (“MSC” or “MSCs”), and/or products derived therefrom (e.g., exosomes or growth factors derived therefrom), for preventing, managing, and/or treating various ophthalmic and ocular conditions and/or diseases (e.g., tear hyperosmolarity (THO), dry eye discomfort, tear hyperosmolarity, tear hyperosmolarity-induced pathological changes in the eyes of patients suffering from dry eye discomfort, dry eye disease (DED)). Specifically described herein is an MSC-derived biological product d-MAPPS™ regenerative biologics platform technology (“d-MAPPS”), which contains large numbers of MSC-sourced growth factors, anti-inflammatory cytokines and chemokines. Notably, the acronym d-MAPPS in d-MAPPS™ regenerative biologics platform technology stands for “derived Multiple Allogeneic Proteins Paracrine Signaling”. In vitro, d-MAPPS™ regenerative biologics platform technology efficiently inhibits proliferation of activated human peripheral blood mononuclear cells (“pbMNCs”). d-MAPPS™ regenerative biologics platform technology further suppresses production of inflammatory cytokines and promotes secretion of immunosuppressive factors in pbMNCs. In addition, d-MAPPS™ regenerative biologics platform technology favors development of tolerogenic and regulatory phenotype in activated monocytes and lymphocytes, indicating its potential for therapeutic use in various diseases, including one or more of the eye diseases described herein (e.g., THO, dry eye discomfort, tear hyperosmolarity, tear hyperosmolarity-induced pathological changes in the eyes of patients suffering from dry eye discomfort, DED, as well as other diseases (e.g., various cancers).

In at least one embodiment, d-MAPPS™ regenerative biologics platform technology includes immunostimulatory molecules (e.g., IL-27 and CXCL16) that enhance T-cell driven immune responses. In some embodiments, a method for prevention and treatment of a disease (e.g., one or more eye conditions and/or diseases including, but not limited to, THO) is disclosed, including altering the response of endogenous immune cells in the subject provided, comprising administering to the subject an effective amount of d-MAPPS™ regenerative biologics platform technology, thereby altering the response of endogenous immune cells (e.g., dendritic cells, macrophages, natural killer cells, T cells, and the like) in the subject. In embodiments, administration of effective amount of d-MAPPS™ regenerative biologics platform technology improves one or more symptoms of one or more eye diseases in the subject. In some embodiments, d-MAPPS™ regenerative biologics platform technology may be administered in combination with one or more agents selected from the group consisting of d-MAPPS-associated MSCs, placenta tissue-derived MSCs, antimicrobial agents, analgesic agents, local anesthetic agents, anti-inflammatory agents, anti-oxidant agents, immunosuppressant agents, anti-allergenic agents, enzyme cofactors, essential nutrients, growth factors, and combinations thereof.

In some embodiments, a pharmaceutical composition comprising d-MAPPS™ regenerative biologics platform technology (also referred to herein as “d-MAPPS pharmaceutical composition”) is disclosed. Such d-MAPPS pharmaceutical compositions may be formulated in various formulations, including, for example, as one or more liquid solutions (also referred to herein as “d-MAPPS solution” or “d-MAPPS solutions”). At least one such d-MAPPS solution is suitable for administration to the eyes of a subject as eye drops. Other formulations (e.g., gels, solids) are possible. The d-MAPPS pharmaceutical compositions (e.g., one or more d-MAPPS solutions) may comprise, for instance, one or more types of MSCs, one or more types of exosomes derived from one or more types of MSCs (“MSC-Exos”) (e.g., exosomes generated ex vivo from mesenchymal stem cells, wherein the mesenchymal stem cells may be, for instance, placental tissue-derived mesenchymal stem cells), one or more MSC-sourced growth factors and/or immunoregulatory proteins, and/or sterile de-cellularized human amniotic fluid (D-HAF). Such exosomes may be used as a delivery vehicle for one or more MSC-sourced biological molecules (e.g., anti-tumorigenic miRNAs, messenger RNAs (mRNAs), enzymes, cytokines, chemokines, growth factors, immunomodulatory factors, small-molecule drugs, proteins, and combinations thereof. The d-MAPPS pharmaceutical compositions (e.g., one or more d-MAPPS solutions) may also comprise one or more osmoprotectants to further address, remedy, and/or alleviate THO. The d-MAPPS pharmaceutical compositions (e.g., one or more d-MAPPS solutions) may further comprise one or more pharmaceutically acceptable excipients. The d-MAPPS pharmaceutical compositions (e.g., one or more d-MAPPS solutions) may also comprise one or more agents selected from the group consisting of adjuvants, antioxidants, anti-inflammatory agents, growth factors, neuroprotective agents, antimicrobial agents, local anesthetics, and combinations thereof.

In at least one example, the d-MAPPS pharmaceutical composition (e.g., one or more d-MAPPS solutions) includes a formation for topical application to the eye for the treatment of THO, DED, dry eye discomfort, tear hyperosmolarity, and/or other induced pathological changes in the eyes of patients suffering from dry eye discomfort. Further conditions, diseases, and/or injuries that may be treated include Sjogren's syndrome, cataracts, burns, and injuries to the eye tissues. The aforementioned composition may, in some instances, contain a human amniotic fluid formulation. Such human amniotic fluid formulation may be a specifically formulated, sterile filtered de-cellularized human amniotic fluid. The d-MAPPS pharmaceutical composition (e.g., one or more d-MAPPS solutions) may be applied directly to the eye(s), preferably as a liquid ocular solution, much like a common liquid eye drops, lubricant, or gel. The d-MAPPS pharmaceutical composition (e.g., one or more d-MAPPS solutions) can alleviate or prevent at least one symptom of a number of ocular injuries and diseases, including dry eye disease, dry eye discomfort, tear hyperosmolarity, tear hyperosmolarity-induced pathological changes in the eyes of patients suffering from dry eye discomfort, Sjogren's syndrome, and burns or injuries, corneal neovascular disorders, corneal opacities (including corneal haze), and prolonged redness and inflammation of the eye(s).

In the specific, non-limiting example of DED, decreased tear secretion or altered tear composition leads to tear film instability/imbalance which, in DED patients, results in the abnormally rapid breakup of the tear film after blinking. Numerous structural changes in epithelial cells and mucin-producing goblet cells develop as a consequence of exposition of these cells to the hyperosmolar tears. Tear hyperosmolarity can cause and/or induce oxidative stress, disruption of DNA repair systems, and DNA damage, particularly in the cells of the ocular surface and lacrimal system. This can result in, for instance, cell apoptosis. An injury of the lacrimal glands may result in decreased tear secretion, enabling the creation of a positive feedback loop that leads to DED progression and aggravation. Eye drops containing d-MAPPS (e.g., one or more d-MAPPS solutions) can alleviate THO and restore tear homeostasis at the corneal surface. Such drops can therefore break the aforementioned positive feedback loop and relieve eye pain, irritation, discomfort, and vision disturbance in DED patients. In at least one embodiment, the d-MAPPS solution is a hypotonic solution enriched with osmoprotectants that address the hyperosmolarity of a tear film. These osmoprotectants help support tear stability and assist in relieving eye dryness in DED patients.

In at least one embodiment, a d-MAPPS pharmaceutical composition (e.g., a d-MAPPS solutions) may include a sterile de-cellularized human amniotic fluid (D-HAF), preferably diluted with a pharmaceutically accepted carrier, and typically administered using a standard eye dropper apparatus. D-HAF contains over 300 human growth factors. D-HAF is devoid of amniotic stem cells and elements of micronized membrane or chorion particles. The dilution ratio of the D-HAF is dependent on the severity of the disorder or injury. For example, early to moderate dry eye or chronic redness, surface inflammation and, intraocular inflammation may be best treated with a low concentration, whereas Sjogren's syndrome, severe Dry Eye, a corneal neovascular disorder, or corneal opacity will typically utilize a higher concentration of D-HAF. Daily applications of a d-MAPPS pharmaceutical composition (e.g., one or more d-MAPPS solutions containing D-HAF) can deliver a sustainable level of beneficial growth factors.

D-HAF is prepared from human amniotic fluid (“AF”) from which the amniotic stem cells and particulate matter have been removed. In at least one embodiment, the process includes separating the cells from the AF using centrifugation and utilizing a series of filtration devices to remove all remaining cells and bioburden. Each lot is tested for bioburden and is certified sterile to contain <1 harmful organisms. The purified fluid is sterilized without the use of harsh terminal irradiation, e-beam, or Ethylene Oxide (EO).

Methods for treating or preventing an ocular disease, disorder, or injury of the eye using one or more of the described d-MAPPS pharmaceutical compositions (e.g., one or more d-MAPPS solutions) are described. In some embodiments, the aforementioned compositions are administered with a pharmaceutically acceptable carrier. In some embodiments, such compositions are administered as a solution, suspension, ointment, or gel, with or without an implant. In some embodiments, the disorders associated with the eye that are suitable for treatment include THO, THO-induced changes in patients suffering from dry eye discomfort, DED, dry eye discomfort, ocular burns, tears or injury to the eye or associated structures, corneal neovascular disorders, corneal opacities (including corneal haze), ocular blast injuries, eye infections, eye surgeries, drug-induced eye conditions, and prolonged redness and inflammation of the eye. In some embodiments, the disorders to be treated include amoebic keratitis, fungal keratitis, bacterial keratitis, viral keratitis, onchorcercal keratitis, bacterial keratoconjunctivitis, viral keratoconjunctivitis, corneal dystrophic diseases, Fuchs' endothelial dystrophy, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, anterior uveitis, posterior uveitis (including toxoplasmosis), pan-uveitis, an inflammatory disease of the vitreous or retina, endophthalmitis prophylaxis and treatment, macular edema, macular degeneration, age related macular degeneration, proliferative and non-proliferative diabetic retinopathy, hypertensive retinopathy, an autoimmune disease of the retina, primary and metastatic intraocular melanoma, other intraocular metastatic tumors, open angle glaucoma, closed angle glaucoma, pigmentary glaucoma, and combinations thereof. Other disorders include injury, burns, or abrasion of the cornea, cataracts and age related degeneration of the eye or vision associated therewith.

Methods for treating, or preventing a disease, disorder, or injury of the eye using one or more d-MAPPS pharmaceutical compositions (e.g., one or more d-MAPPS solutions) in combination with one or more therapeutic, prophylactic or diagnostic agents are also described. In some embodiments, one or more d-MAPPS pharmaceutical compositions (e.g., one or more d-MAPPS solutions) is administered prior to, in conjunction with, subsequent to, or alternation with treatment with one or more therapeutic, prophylactic or diagnostic agents. In some embodiments, the one or more therapeutic, prophylactic or diagnostic agents are selected from the group consisting of an anti-glaucoma agent, an anti-angiogenesis agent, an anti-infective agent, an anti-inflammatory agent, an analgesic agent, a local anesthetic, a growth factor, an immunosuppressant agent, an anti-allergic agent, an anti-oxidant, a cytokine, and combinations thereof. In some embodiments, the one or more diagnostic agents include paramagnetic molecules, fluorescent compounds, magnetic molecules, and radionuclides, x-ray imaging agents, contrast media.

These and further and other objects and features of the invention are apparent in the disclosure, which includes the above and ongoing written specification, as well as the drawings.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 shows various non-limiting effects of specific hormones on tear osmolarity, according to at least one embodiment of the disclosure.

FIG. 2 shows various non-limiting effects of tear hyperosmolarity, according to at least one embodiment of the disclosure.

FIG. 3 shows various non-limiting effects of the generation and/or activation of Th1 and/or Th17 cells, according to at least one embodiment of the disclosure.

FIG. 4 shows various non-limiting ways that tear hyperosmolarity can modulate the phenotype and/or function of neutrophils, natural killer cells, and/or natural killer T cells, according to at least one embodiment of the disclosure.

FIG. 5 shows various non-limiting processes by which tear hyperosmolarity initiates and/or aggravates dry eye disease, according to at least one embodiment of the disclosure.

### DETAILED DESCRIPTION

The present invention is more fully described below with reference to the accompanying figures. The following description is exemplary in that several embodiments are described (e.g., by use of the terms “preferably,” “for example,” or “in one embodiment”); however, such should not be viewed as limiting or as setting forth the only embodiments of the present invention, as the invention encompasses other embodiments not specifically recited in this description, including alternatives, modifications, and equivalents within the spirit and scope of the invention. Further, the use of the terms “invention,” “present invention,” “embodiment,” and similar terms throughout the description are used broadly and not intended to mean that the invention requires, or is limited to, any particular aspect being described or that such description is the only manner in which the invention may be made or used. Additionally, the invention may be described in the context of specific applications; however, the invention may be used in a variety of applications not specifically described.

The embodiment(s) described, and references in the specification to “one embodiment,” “an embodiment,” “an example embodiment,” etc., indicate that the embodiment(s) described may include a particular feature, structure, or characteristic. Such phrases are not necessarily referring to the same embodiment. When a particular feature, structure, or characteristic is described in connection with an embodiment, persons skilled in the art may affect such feature, structure, or characteristic in connection with other embodiments whether or not explicitly described.

In the several figures, like reference numerals may be used for like elements having like functions even in different drawings. The embodiments described, and their detailed construction and elements, are merely provided to assist in a comprehensive understanding of the invention. Thus, it is apparent that the present invention can be carried out in a variety of ways and does not require any of the specific features described herein. Also, well-known functions or constructions are not described in detail since they would obscure the invention with unnecessary detail. Any signal arrows in the drawings/figures should be considered only as exemplary, and not limiting, unless otherwise specifically noted. Further, the description is not to be taken in a limiting sense, but is made merely for the purpose of illustrating the general principles of the invention, since the scope of the invention is best defined by the appended claims.

It will be understood that, although the terms “first,” “second,” etc. may be used herein to describe various elements, these elements should not be limited by these terms. These terms are only used to distinguish one element from another. Purely as a non-limiting example, a first element could be termed a second element, and, similarly, a second element could be termed a first element, without departing from the scope of example embodiments. As used herein, the term “and/or” includes any and all combinations of one or more of the associated listed items. As used herein, “at least one of A, B, and C” indicates A or B or C or any combination thereof. As used herein, the singular forms “a,” “an,” and “the” are intended to include the plural forms as well, unless the context clearly indicates otherwise. It should also be noted that, in some alternative implementations, the functions and/or acts noted may occur out of the order as represented in at least one of the several figures. Purely as a non-limiting example, two figures shown in succession may in fact be executed substantially concurrently or may sometimes be executed in the reverse order, depending upon the functionality and/or acts described or depicted.

As used herein, ranges are used herein in shorthand, so as to avoid having to list and describe each and every value within the range. Any appropriate value within the range can be selected, where appropriate, as the upper value, lower value, or the terminus of the range.

Unless indicated to the contrary, numerical parameters set forth herein are approximations that can vary depending upon the desired properties sought to be obtained. At the very least, and not as an attempt to limit the application of the doctrine of equivalents to the scope of any claims, each numerical parameter should be construed in light of the number of significant digits and ordinary rounding approaches.

The words “comprise,” “comprises,” and “comprising” are to be interpreted inclusively rather than exclusively. Likewise, the terms “include,” “including,” and “or” should all be construed to be inclusive, unless such a construction is clearly prohibited from the context. The terms “comprising” or “including” are intended to include embodiments encompassed by the terms “consisting essentially of” and “consisting of.” Similarly, the term “consisting essentially of” is intended to include embodiments encompassed by the term “consisting of.” Although having distinct meanings, the terms “comprising,” “having,” “containing,” and “consisting of” may be replaced with one another throughout the description of the invention.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Terms such as, among others, “about,” “approximately,” “approaching,” or “substantially,” mean within an acceptable error for a particular value or numeric indication as determined by one of ordinary skill in the art, which depends in part on how the value is measured or determined. The aforementioned terms, when used with reference to a particular non-zero value or numeric indication, are intended to mean plus or minus 10% of that referenced numeric indication. As an example, the term “about 4” would include a range of 3.6 to 4.4. All numbers expressing dimensions, velocity, and so forth used in the specification are to be understood as being modified in all instances by the term “about.” Accordingly, unless indicated to the contrary, the numerical parameters set forth herein are approximations that can vary depending upon the desired properties sought to be obtained. At the very least, and not as an attempt to limit the application of the doctrine of equivalents to the scope of any claims, each numerical parameter should be construed in light of the number of significant digits and ordinary rounding approaches.

“Typically” or “optionally” means that the subsequently described event or circumstance may or may not occur, and that the description includes instances where said event or circumstance occurs and instances where it does not.

Wherever the phrase “for example,” “such as,” “including” and the like are used herein, the phrase “and without limitation” is understood to follow unless explicitly stated otherwise.

### Definitions

The following is a non-exhaustive and non-limiting list of terms used herein and their respective definitions.

The terms “agent” or “active agent,” which are used interchangeably herein, refer to a physiologically or pharmacologically active substance that acts locally and/or systemically in a subject's body. An “agent” or “active agent” is a compound or substance that is administered to an individual for the treatment (e.g., therapeutic agent, cancer therapeutic agent, and the like), prevention (e.g., prophylactic agent), or diagnosis (e.g., diagnostic agent) of a disease or disorder. Such agents may also include therapeutics that prevent or alleviate symptoms, such as, for instance, symptoms associated with one or more eye disorders or treatments for such disorders. “Ophthalmic drug” or “ophthalmic active agent,” as used herein, refers to an agent that is administered to a patient to alleviate, delay onset of, and/or prevent one or more symptoms of a disease or disorder of the eye, or a diagnostic agent useful for imaging or otherwise assessing the eye.

The term “administering” or “administration” refers to providing or giving a subject one or more agents and/or formulations, such as one or more d-MAPPS pharmaceutical compositions, either alone or in conjunction with any other compound and/or agent (including, e.g., prophylactic or therapeutic agents), by any effective route. Exemplary routes of administration include, but are not limited to, injection (such as, e.g., subcutaneous, subdermal, intramuscular, intradermal, intraperitoneal, intracerebroventricular, intraosseous, intratumoral, intraprostatic, and intravenous), transdermal, intranasal, oral, vaginal, rectal, and inhalation.

The term “amniotic factor” generally refers to one or more compounds naturally present in the amniotic fluid. These include, for example, carbohydrates, proteins and peptides (e.g., enzymes, hormones), lipids, metabolic substrates and products (e.g., lactate, pyruvate), and electrolytes.

The term “antigen” refers to a compound, composition, and/or substance that can stimulate the production of antibodies or an immune response in a subject, including compositions that are injected or absorbed into a subject. An “antigen” may react with the products of specific humoral and/or cellular immunity, including, for example, those induced by heterologous antigens.

The term “biocompatible” or “biologically compatible,” as used herein, generally refers to materials that are, along with any metabolites or degradation products thereof, generally non-toxic to the recipient, and do not cause any significant adverse effects to the recipient. Generally speaking, biocompatible materials are materials which do not elicit a significant inflammatory or immune response when administered to a patient or subject.

The term “biodegradable polymer,” as used herein, generally refers to a polymer that will degrade or erode by enzymatic action and/or hydrolysis under physiologic conditions to smaller units or chemical species that are capable of being metabolized, eliminated, or excreted by the subject. The degradation time is a function of polymer composition, morphology, such as porosity, particle dimensions, and environment.

The term “cancer” refers to a class of diseases or conditions in which abnormal cells divide without control and can invade nearby tissues. A malignant cancer is one in which a group of tumor cells display one or more of uncontrolled growth (e.g., division beyond normal limits), invasion (e.g., intrusion on and destruction of adjacent tissues), and/or metastasis (e.g., spread to other locations in the subject's body via lymph or blood). As used herein, the terms “metastasis” or “metastasize” refer to the spread of cancer from one part of the body to another. A tumor formed by cells that have spread is called a “metastatic tumor” or a “metastasis.” The metastatic tumor contains cells that are similar to those in the original tumor (i.e., the tumor at the primary site of tumor growth). A “cancer cell” or “tumor cell” refers to an individual cell of a cancerous growth or tissue. A “tumor” refers generally to a swelling or lesion formed by an abnormal growth of cells, which may be benign, pre-malignant, or malignant. Most cancers form tumors, but some, e.g., leukemia, and some blood cancers, do not necessarily form tumors. For those cancers that form tumors, the terms “cancer,” “cancer cell,” “tumor,” and “tumor cell” are used interchangeably. The amount of a tumor in a given subject is the “tumor burden,” which can be measured as the number, volume, and/or weight of the tumor.

The term “combination therapy” refers to the administration of different compounds, agents, and/or individual therapies in a sequential and/or simultaneous manner. Individual elements of a “combination therapy” may be administered at different times and/or by different routes, but act in combination to provide a beneficial effect on the subject.

The term “compound” refers to a substance formed from one or more chemical elements, arranged together in any proportion or structural arrangement. The one or more chemical elements may be either naturally occurring and/or non-naturally occurring. As used herein, the term “biological compound” refers to a compound of biological origin and/or having one or more effects on a subject's local and/or systemic biological functions. Accordingly, “compounds” or “biological compounds” include, as non-limiting examples, various proteins (e.g., growth factors, hormones, enzymes), nucleic acids, and pharmaceutical products (e.g., drugs, prodrugs). The term “drug” generally refers to a medicine or other substance that has a physiological effect when introduced into a subject. The term “prodrug” generally refers to a biologically and/or chemically inactive compound that can be metabolized by a subject to produce a drug.

The terms “decrease,” “lower,” “lessen,” “reduce,” and “abate,” which are used interchangeably herein, refer generally to the ability of a compound, formulation, or therapy (including those disclosed herein) to produce, elicit, and/or cause a lesser physiological response (e.g., downstream effects) compared to the response caused by a respective control compound, formulation, or therapy. A “decrease” or “reduced” amount is typically a “statistically significant” amount, and may include a decrease that is, for instance, 1.1, 1.2, 1.5, 2, 3, 4, 5, 6, 7, 8, 9, 10, 15, 20, or 30 or more times (e.g., 500, 1000 times) (including all integers and decimal points in between and above 1, e.g., 1.5, 1.6, 1.7. 1.8, etc.).

The term “dendritic cell” refers to a type of specialized antigen-presenting cell (“APC”) involved in innate and/or adaptive immunity. Dendritic cells may also be referred to herein as “DC” or “DCs.” Dendritic cells may be present in the tumor microenvironment, and these are referred to as “tumor-associated dendritic cells” (“tDC” or “tDCs”).

The terms “effective amount” or “therapeutically effective amount,” which are used interchangeably herein, refer to the amount of an agent (e.g., including one or more d-MAPPS pharmaceutical compositions described herein) that is sufficient to effect beneficial or desired therapeutic result, including clinical results. An “effective amount” may vary depending upon one or more of: the subject and disease condition being treated, the sex, weight and age of the subject, the severity of the disease condition, the manner of administration, the ability of one or more formulations to elicit a desired response in the subject, and the like. The beneficial therapeutic effect can include, but is not limited to, enablement of diagnostic determinations; prevention of disease or tumor formation; amelioration of a disease, symptom, disorder, and/or pathological condition; reducing or preventing the onset of a disease, symptom, disorder, and/or pathological condition; and generally counteracting a disease, symptom, disorder, and/or pathological condition. The term “effective amount” includes an amount that is effective to “treat” a subject (e.g., a patient or individual), including an amount effective to alleviate, delay onset of, and/or prevent one or more symptoms, particularly of a disease or disorder of the eye. When a therapeutic amount is indicated, the precise amount of one or more formulations described in the present disclosure to be administered can be determined by a physician, based on, for instance, considerations such as individual differences in age, weight, extent of the disease or disorder, and/or condition of the subject (individual).

The terms “enhance,” “induce,” “induction,” and “increase,” which are used interchangeably herein, refer generally to the ability of a compound, formulation, or therapy (including those disclosed herein) to produce, elicit, and/or cause a greater physiological response (e.g., downstream effects) compared to the response caused by a respective control compound, formulation, or therapy. An “enhanced” or “increased” amount is typically a “statistically significant” amount, and may include an increase that is, for instance, 1.1, 1.2, 1.5, 2, 3, 4, 5, 6, 7, 8, 9, 10, 15, 20, or 30 or more times (e.g., 500, 1000 times) (including all integers and decimal points in between and above 1, e.g., 1.5, 1.6, 1.7. 1.8, etc.).

The term “growth factor” refers to any compound (e.g., one or more groups of proteins or hormones) that stimulate cellular growth. Generally, growth factors play an important role in promoting cellular differentiation and cell division, and they occur in a wide range of organisms, including humans.

The term “immune cell” refers to any cell of the immune system that has one or more effector functions (e.g., cytotoxic cell killing activity, secretion of cytokines, induction of antibody-dependent cell-mediated cytotoxicity (ADCC), and/or induction of complement-dependent cytotoxicity (CDC)).

The terms “immunologic,” “immunological,” or “immune” response, which are used interchangeably herein, refer to the development of a beneficial humoral (i.e., antibody-mediated) and/or a cellular (e.g., mediated by immune cells, such as antigen-specific T cells, or their secretion products) response directed against an antigen and/or immunogen in a specific subject. Such a response can be an active response induced by administration of an antigen and/or immunogen, or a passive response induced by administration of antibodies or primed T-cells. A cellular immune response is elicited by the presentation of polypeptide epitopes in association with Class I or Class II major histocompatibility complex (MHC) molecules to activate antigen-specific CD4+ healer T cells and/or cos+ cytotoxic T cells. The response may also involve, for instance, activation of monocytes, macrophages, natural killer (NK) cells, basophils, dendritic cells, astrocytes, microglia cells, eosinophils, and/or other components of innate immunity. The presence of a cell-mediated immunological response can be determined by proliferation assays (e.g., CD4+ T cells) or cytotoxic T lymphocyte (CTL) assays. The relative contributions of humoral and cellular responses to the protective or therapeutic effect of an antigen and/or immunogen can be distinguished by, for example, separately isolating antibodies and T cells from an immunized syngeneic animal and measuring the protective or therapeutic effect in a second subject.

The term “implant,” as generally used herein, refers to a polymeric device or element that is structured, sized, or otherwise configured to be implanted, preferably by injection or surgical implantation, in a specific region of the body so as to provide therapeutic benefit by releasing one or more therapeutic, prophylactic or diagnostic agents over an extended period of time at the site of implantation. For example, intraocular implants are polymeric devices or elements that are structured, sized, or otherwise configured to be placed in the eye, preferably by injection or surgical implantation, and to treat one or more diseases or disorders of the eye by releasing one or more therapeutic, prophylactic or diagnostic agents over an extended period. Intraocular implants are generally biocompatible with physiological conditions of an eye and do not cause adverse side effects. Generally, intraocular implants may be placed in an eye without disrupting vision of the eye.

The term “ionizing radiation” refers to radiation, traveling as a particle or electromagnetic wave, that carries sufficient energy to detach electrons from atoms or molecules, thereby ionizing an atom or a molecule. Generally, ionizing radiation is made up of energetic subatomic particles, ions, or atoms moving at high speeds and electromagnetic waves on the high-energy end of the electromagnetic spectrum. Radiation has been demonstrated to induce adaptive immune responses to mediate tumor regression. In addition, the induction of type I interferons (“IFNs”) by radiation is essential for the function of CD8+ T cells. Radiation induces cell stress and causes excess deoxyribonucleic acid (DNA) breaks, indicating that the nucleic acid-sensing pathway likely accounts for the induction of type I IFNs upon radiation. Type I IFN responses in DCs dictate the efficacy of antitumor radiation. In contrast, chemotherapeutic agents and anti-human epidermal growth factor receptor 2 (HER2) antibody treatments have been demonstrated to depend on a distinct immune mechanism to trigger adaptive immune responses. In general, therapeutic radiation-mediated antitumor immunity depends on a proper cytosolic DNA sensing pathway. In at least one embodiment, one or more agents, formulations, and/or methods (e.g., including one or more types of MSCs, either alone or in conjunction with one or more other agents) described herein is administered in combination with radiation therapy.

The term “macrophage” refers to a type of white blood cell of the immune system that engulfs and digests cellular debris, foreign substances, microbes, cancer cells, and the like. These phagocytes include various subtypes (e.g., histiocytes, Kupffer cells, alveolar macrophages, microglia, and others), but all are part of the mononuclear phagocyte system. Besides phagocytosis, macrophages play a critical role in both innate and adaptive immunity by recruiting other endogenous immune cells (e.g., lymphocytes). For example, they are important as antigen presenters to T cells. In humans, dysfunctional macrophages can cause severe diseases (e.g., chronic granulomatous disease) that result in frequent infections. Beyond increasing inflammation and stimulating the immune system, macrophages also play an important anti-inflammatory role and can decrease immune reactions through the release of various compounds (e.g., cytokines). Macrophages that encourage inflammation may be termed “M1 macrophages” because they have the so-called “M1 phenotype,” whereas those that decrease inflammation and encourage tissue repair may be termed “M2 macrophages” because they have the so-called “M2 phenotype.”

The term “mean particle size,” as used herein, generally refers to the statistical mean particle size (diameter) of the particles in a population of particles. The diameter of an essentially spherical particle may refer to the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering.

The term “microparticle,” as used herein, generally refers to a particle having a diameter, such as an average diameter, from about 1 micron to about 100 microns, preferably from about 1 micron to about 50 microns, more preferably from about 1 to about 30 microns. The microparticles can have any shape. Microparticles having a spherical shape are generally referred to as “microspheres.”

The term “molecular weight” generally refers to the relative average chain length of a bulk polymer or protein, unless otherwise specified. In practice, molecular weights can be estimated or characterized using various methods including, for example, gel permeation chromatography (GPC) or capillary viscometry. GPC molecular weights are reported as the weight-average molecular weight (MW), as opposed to the number-average molecular weight (MN). Capillary viscometry provides estimates of molecular weight as the inherent viscosity determined from a dilute polymer solution using a particular set of concentration, temperature, and solvent conditions.

The term “nanoparticle,” as used herein, generally refers to a particle having a diameter, such as an average diameter, from about 10 nanometers (nm) up to but not including about 1 micron, preferably from 100 nm to about 1 micron. The particles can have any shape. Nanoparticles having a spherical shape are generally referred to as “nanospheres.”

The term “parenteral administration” refers to a type of administration by any method other than through the digestive tract or non-invasive topical or regional routes. As a non-limiting example, parenteral administration may include administration to a subject via intravenous, intradermal, intraperitoneal, intrapleural, intratracheal, intraarticular, intrathecal, intramuscular, subcutaneous, subjunctival, injection, and/or infusion.

The term “peptide” refers to a polymer of amino acid residues. The amino acid residues may be naturally occurring and/or non-naturally occurring. The terms “polypeptide,” “peptide,” and “protein” are used interchangeably herein. The terms apply to, for instance, amino acid polymers of one or more amino acid residues, an artificial chemical mimetic of a corresponding naturally occurring amino acid, naturally occurring amino acid polymers, and non-naturally occurring amino acid polymers.

The term “pharmaceutically acceptable,” as used herein, refers to compounds, carriers, excipients, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “subject,” “individual,” or “patient,” which are used interchangeably herein, refer to a vertebrate, such as a mammal (e.g., a human). Mammals include, but are not limited to, murines (e.g., mice), simians, humans, farm animals, sport animals, and pets. In at least one embodiment, the subject is a non-human mammal, such as a monkey or other non-human primate, mouse, rat, rabbit, guinea pig, pig, goat, sheep, dog, cat, horse, or cow. In at least one example, the subject has a tumor, such as a cancer, that can be treated using one or more agents, formulations, and/or methods (e.g., including one or more d-MAPPS pharmaceutical compositions, either alone or in conjunction with one or more other agents) disclosed herein. In at least an additional example, the subject is a laboratory animal/organism, such as, for example, a mouse, rabbit, guinea pig, or rat. In at least a further example, a subject includes, for instance, farm animals, domestic animals and/or pets (e.g., cats, dogs). In at least a still further example, a subject is a human patient that has one or more eye disorders, has been diagnosed with an eye disorder, and/or is at risk of having an eye disorder. A “patient” can specifically refer to a subject that has been diagnosed with a particular disease, condition, and/or indication that can be treated with refers to a subject that has been diagnosed with a particular indication that can be treated with one or more agents, formulations, and/or methods (e.g., including one or more d-MAPPS pharmaceutical compositions, either alone or in conjunction with one or more other agents) disclosed herein.

The term “topical administration” refers to a type of non-invasive administration to the skin, orifices, and/or mucosa of a subject. Topical administrations can be administered locally; that is, they are capable of providing a local effect in the region of application without systemic exposure. Topical formulations can, however, provide one or more systemic effects via, e.g., adsorption into the blood stream of the individual. Routes of topical administration include, but are not limited to, cutaneous and transdermal administration, buccal administration, intranasal administration, intravaginal administration, intravesical administration, ophthalmic administration, pulmonary administration, and rectal administration.

The terms “treating,” “treatment,” and “therapy” refer, either individually or in any combination, to any success or indicia of success in the attenuation or amelioration of an injury, disease, symptom, disorder, pathology, and/or condition, and/or pathological condition, including any objective or subjective parameter such as, for instance, abatement, remission, diminishing of symptoms or making the condition more tolerable to the patient, slowing the rate of degeneration or decline, making the final point of degeneration less debilitating, improving a subject's physical or mental well-being, and/or prolonging the length of survival. Treatment does not necessarily indicate complete eradication or cure of the injury, disease, symptom, disorder, pathology, and/or condition, and/or pathological condition, or any associated symptom(s) thereof. The treatment may be assessed by one or more objective or subjective parameters, including, for example, the results of a physical examination, blood and other clinical tests (e.g., imaging), and the like. In at least one example, treatment with the disclosed one or more agents, formulations, and/or methods (e.g., including one or more d-MAPPS pharmaceutical compositions, either alone or in conjunction with one or more other agents) results in a clinical improvement in one or more eye diseases in a subject. Further, unless otherwise noted, technical terms are generally used according to conventional usage. Aspects of the disclosed methods employ, unless indicated specifically to the contrary, conventional methods of chemistry, biochemistry, organic chemistry, molecular biology, microbiology, recombinant DNA techniques, genetics, immunology, and/or cell biology, many of which are described below solely for the purpose of illustration. Such techniques are explained fully in technical literature sources. General definitions of common terms in the aforementioned fields, including, for instance, molecular biology, may be found in references such as, e.g., Krebs et al., Lewin's Genes X, Jones & Bartlett Learning (2009) (ISBN 0763766321); Rédei, Encyclopedic Dictionary of Genetics, Genomics, Proteomics and Informatics (3rd ed.), Springer (2008) (ISBN: 1402067532); Ausubel et al., Current Protocols in Molecular Biology, John Wiley and Sons (updated July 2008) (ISBN: 047150338X); Ausubel et al., Short Protocols in Molecular Biology: A Compendium of Methods from Current Protocols in Molecular Biology (2nd ed.), Wiley-Interscience (1989) (ISBN 0471514705); Glover, et al., DNA Cloning: A Practical Approach, Vol. I-II, Oxford University Press (1985) (ISBN 0199634777); Anand et al., Techniques for the Analysis of Complex Genomes, Academic Press (1992) (ISBN 0120576201); Hames et al., Transcription and Translation: A Practical Approach, Oxford University Press (1984) (ISBN 0904147525); Perbal et al., A Practical Guide to Molecular Cloning (2nd ed.), Wiley-Interscience (1988) (ISBN 0471850713); Kendrew et al., Encyclopedia of Molecular Biology, Wiley-Blackwall (1994) (ISBN 0632021829); Meyers et al., Molecular Biology and Biotechnology: A Comprehensive Desk Reference, Wiley-VCH (1996) (ISBN 047118571X); Harlow et al., Antibodies: A Laboratory Manual, Cold Spring Harbor Laboratory Press (1988) (ISBN 0879693746); Coligan et al., Current Protocols in Immunology, Current Protocols (2002) (ISBN 0471522767); Annual Review of Immunology; articles and/or monographs in scientific journals (e.g., Advances in Immunology); and other similar references.

### Dry Eye Disease and Dry Eye Discomfort

Dry eye disease (DED), also known as keratoconjunctivitis sicca or dysfunctional tear syndrome, is a common, multifactorial disease of the lacrimal system and ocular surface. DED is often characterized by a deficiency in the quality and/or quantity of the tear fluid.

Since the ocular surface is highly exposed to environmental hazards, efficient tear production and optimal tear turnover is essential for appropriate eye function. The tear film, lacrimal glands (main and accessory lacrimal glands), meibomian glands, mucin-producing goblet cells, ocular surface secretory cells, lacrimal outflow pathways, and corneal and conjunctival epithelial cells work together and function as a lacrimal functional unit (LFU). For instance, the meibomian glands are oil-producing glands positioned along the edge of the eyelids that create an oily layer outside of the tear film, keeping tears from drying up too quickly. The aforementioned LFU has several functions, including maintenance of tear film, protection of the transparency of the cornea, and protection of the integrity of the ocular surface. Importantly, the LFU is not an isolated system, and it functions in association with the nervous and endocrine systems. Damage to any LFU component and/or development of neural and endocrine disease (e.g., the dysfunction of sensory and motor nerves, hormone imbalance) can destabilize the tear film and lead to the development of dry eye. An unbalanced tear film is not able to provide sufficient nourishment or protection to the ocular surface and, therefore, usually results in permanent damage of the corneal nerve fibers and corneal and conjunctival epithelial cells.

Dry eye disease is often classified into two primary subtypes: aqueous tear-deficient dry eye (ADDE), characterized by the inefficiency or inability of the lacrimal glands to produce tears, and evaporative dry eye (EDE), typically attributed to excessive evaporation of the tear fluid. ADDE may have an autoimmune origin or else can be attributed to a compromise in the LFU integrity. EDE is the more common form of dry eye disease and is frequently associated with meibomian gland dysfunction (MGD). MGD is often characterized by the modification or reduction of tear fluid lipids; as a result, the integrity and quality of the tear fluid may be compromised. Although traditionally dry eye disease has been classified into these two subtypes, there is considerable overlap between them. As such, dry eye disease is most often characterized as a “hybrid” or “mixed” form of these two subtypes, where each subtype adopts some clinical features of the other, initiating and exacerbating its pathology.

The multifactorial nature of dry eye disease involves several inter-related underlying pathologies, including the loss of homeostasis, chronic eye inflammation, and instability and hyperosmolarity of the tears. This can lead to neurosensory dysfunction and visual disturbance. Moreover, these detrimental events can create a “pathological loop” that promotes the progression and aggravation of dry eye disease. DED is usually manifested by dryness, grittiness, scratchiness, soreness, irritation, burning, watering, foreign body sensations, eye fatigue, and/or reduced functional visual acuity. Since significantly impaired performance of vision-dependent daily activities diminishes the quality of life of dry eye disease patients, a better understanding of the pathological steps in dry eye disease pathogenesis is of crucial importance for appropriate dry eye disease treatment.

### Tear Hyperosmolarity (THO) in the Development, Progression, and Aggravation of Dry Eye Disease

Tear hyperosmolarity (also referred to herein as “THO”) plays a crucially vital role in the development and/or progression of DED. However, elevated tear osmolarity is not necessarily associated with aggravation of DED-related signs and symptoms. Further, certain DED patients can exhibit a statistically significant reduction in tear osmolarity after administration of various fatty acids (e.g., omega-3 fatty acids and, in particular, oral re-esterified omega-3 fatty acids). Such administration may result in improved tear breakup times and/or improved omega-3 fatty acid index levels, indicating good absorption. Additionally, these patients may show a significant reduction in Ocular Surface Disease Index (OSDI) symptom scores and matrix metalloproteinase-9 (MMP-9) positivity. Thus, the role of tear osmolarity in the pathogenesis of DED is an important area of investigation, along with the molecular mechanisms responsible for the development of THO-induced pathological changes in the eyes of DED patients.

### Molecular Mechanisms Responsible for THO Development

Generally, tears are a lacrimal gland-sourced salty solution that lubricates and protects various structures and surfaces (e.g., the corneal epithelium and the inner surface of the eyelids) from foreign particles and/or infectious agents. THO can be caused by alterations in tear composition and/or production in cases of poor aqueous tear flow, high tear evaporation, or a combination of these occurrences.

The composition of tears may vary based on their intended purpose. For instance, basal tears contain water, electrolytes, and anti-bacterial proteins (e.g., lysozyme, lactoferrin, and/or lipocalin) which destroy bacterial cell walls and/or bind iron, making it unavailable to any invading pathogens. Emotional tears, on the other hand, contain higher levels of stress hormones and other chemicals (e.g., leucine, enkephalin), which may reduce pain and/or improve mood. Additionally, emotional tears can contain prolactin, which promotes the synthesis of immunoglobulin A (IgA) in plasma cells and enhances anti-bacterial humoral immune responses in the eyes. Alterations in the concentration of electrolytes, proteins, and other solutes in tears can lead to changes in tear osmolarity and can represent one of the first indicators of lacrimal gland dysfunction.

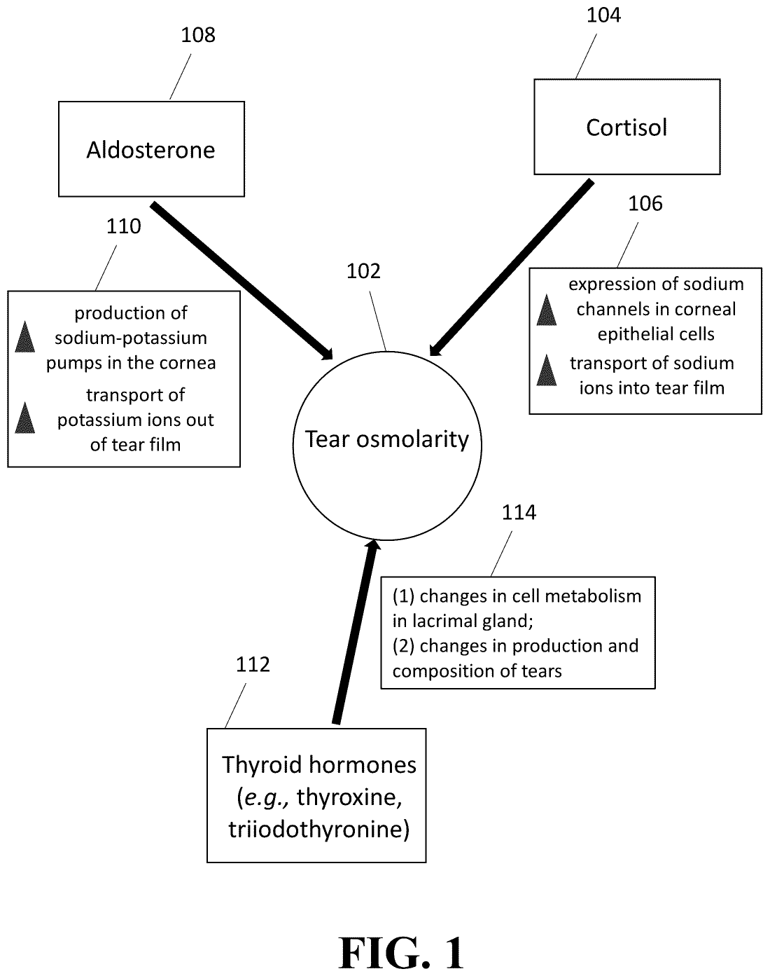

Further, hormones (e.g., cortisol, aldosterone, thyroid hormones), medications (e.g., antihistamines, beta-blockers, diuretics, isotretinoin, anti-cholinergics), and/or environmental factors can affect tear osmolarity and can regulate tear-dependent eye protection. Turning now to FIG. 1, various non-limiting effects of specific hormones on tear osmolarity (block 102) are shown. Cortisol, shown at block 104, can increase tear osmolarity by enhancing the expression of sodium channels in corneal epithelial cells, which can lead to an increase in the transport of sodium ions into the tear film, both shown at block 106. Aldosterone, shown at block 108, can increase tear osmolarity by increasing the production of sodium-potassium pumps in the cornea, which can lead to an increase in the transport of potassium ions out of the tear film, both shown at block 110. Additionally, thyroid hormones (e.g., thyroxine (T4) and triiodothyronine (T3)), shown at block 112, can affect tear osmolarity by affecting the metabolism of cells in the lacrimal gland, which can influence the production and composition of tears, both shown at block 114. Indeed, the increased production of thyroid hormones in overactive thyroid glands can result in increased tear production and decreased tear osmolarity, since the metabolism of cells in the lacrimal gland is increased. Therefore, epiphora, or watery eyes, is frequently observed in patients with hyperthyroidism. On the other hand, patients with hypothyroidism, or an underactive thyroid gland, may have decreased tear production and increased tear osmolarity, since the metabolism of cells in the lacrimal gland is decreased. Since THO can induce pathological changes in the cornea, conjunctiva, and/or lacrimal gland, and can further elicit one or more detrimental immune responses in the eye, THO is considered an important step in the development of DED.

### THO-Related Pathological Changes in the Eyes of DED Patients

Generally, tear osmolarity (e.g., when measured from the lower meniscus) ranges from approximately 300 to 310 milliosmoles (mOsM)/kg in healthy eyes. The interaction of tear production, evaporation, and drainage determines tear osmolarity. However, DED patients, with or without tear volume reduction, tend to have higher evaporation rates than healthy subjects. High evaporation rates can, in turn, eventually result in tear hypertonicity. Although a DED patient may have tear hyperosmolarity as high as 360 mOsM/kg, the current cutoff for diagnosing DED is 316 mOsM/kg.

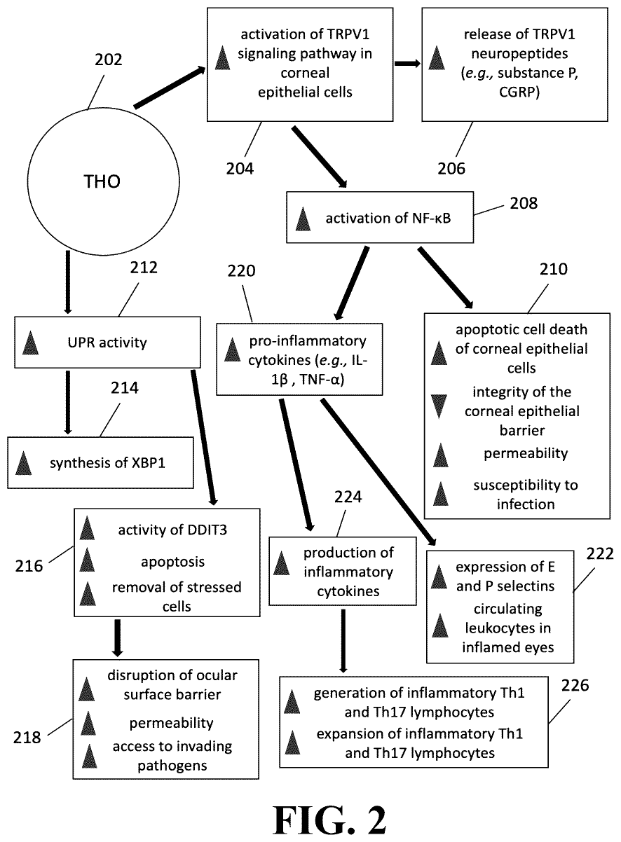

Osmoregulation, which protects the ocular surface epithelia and maintains normal vision, is affected when the osmolarity of the tears is greater than that of the epithelial cells. As a result, THO is considered as an initial, important step in the development, progression, and/or aggravation of DED. Turning now to FIG. 2, various non-limiting effects of THO (shown at block 202) are shown. THO can result in the induction of activation of the transient receptor potential vanilloid 1 (TRPV1) signaling pathway in corneal epithelial cells, shown at block 204. TRPV1 is a cation channel that plays an important role in the regulation of pain and inflammation. THO-dependent activation of TRPV1 promotes the release of neuropeptides (e.g., substance P and calcitonin gene-related peptide (CGRP)), shown at block 206. Such neuropeptides play an important role in the transmission of pain signals. In addition, THO-based induction of TRPV1 signaling in corneal epithelial cells results in the activation of nuclear factor kappa B (NF-κB), shown at block 208. THO-dependent activation of NF-κB in corneal epithelial cells has an important pathogenic role in the development and/or progression of DED. Specifically, by triggering NF-κB signaling, THO induces various effects shown at block 210, including, (1) apoptotic cell death of corneal epithelial cells, (2) disruption of integrity of the corneal epithelial barrier, (3) increased permeability, and/or (4) increased susceptibility to infection.

Accordingly, in line with the above, it is possible for a hyperosmotic environment to cause apoptosis of corneal and conjunctival epithelial cells by inducing dysfunction of the endoplasmic reticulum (ER). The ER is a cellular organelle that contains chaperones and folding enzymes that facilitate the assembly of newly synthesized proteins. This prevents misfolding and aggregation of such newly synthesized proteins. A hyperosmotic microenvironment can affect the trafficking of the secretory cargo in the ER and attenuates its capacity to fold proteins. Accumulation of unfolded proteins activates a stress-related signaling cascade, called unfolded protein response (UPR), which transiently attenuates translation and increases protein folding in the ER of stressed epithelial cells. THO can therefore increase UPR activity, shown at block 212. This increased UPR activity can enhance the synthesis of transcription factor X-box binding protein 1 (XBP1), shown at block 214. XBP 1 directs the expression of chaperones and folding enzymes to restore homeostasis. If THO induces severe ER stress which cannot be attenuated by XBP1, the UPR can increase the activity of DNA-damage-inducible transcript 3 protein (DDIT3), initiating apoptosis and removal of stressed corneal and conjunctival epithelial cells, shown at block 216. The THO-dependent induction of ER-stress-associated programmed death of these cells can therefore result in the disruption of the ocular surface epithelial barrier, increasing permeability and access for invading pathogens in the eyes of DED patients, shown at block 218.

Additionally, the THO-dependent activation of NF-κB in corneal epithelial cells, as shown at block 208, can be responsible for a massive influx of circulating leukocytes in inflamed eyes of DED patients. THO-exposed corneal epithelial cells may highly express adhesion molecules and massively produce pro-inflammatory cytokines (e.g., interleukin (IL)-1β and tumor necrosis factor alpha (TNF-α)), shown at block 220. These cytokines attract circulating immune cells to the inflamed corneas and lacrimal glands of DED patients. Upon binding to their receptors, IL-1β and TNF-α can induce an increased expression of E and P selectins on the membrane of endothelial cells, enabling adhesion, recruitment, and/or influx of circulating leukocytes in inflamed eyes, shown at block 222. Additionally, IL-1β and TNF-α can stimulate enhanced production of inflammatory cytokines in eye-infiltrated immune cells, shown at block 224, thereby creating a “positive inflammatory loop” that leads to the progression of DED. For instance, in dendritic cells (DCs), macrophages, and T lymphocytes, IL-1β and TNF-α can activate several kinases (including, but not limited to, mixed lineage kinase (MLK)-2/-3, transforming growth factor β-activated kinase 1 (TAK1), mitogen-activated protein kinase kinase (MKK)-1, apoptosis signal-regulating kinase 1 (ASK1), MKK7, and Jun N-terminal kinase (JNK)) that phosphorylate and activate transcriptional factors (e.g., c-Jun and ATF2) that translocate to the nucleus to induce enhanced expression of various compounds and/or cytokines (e.g., IL-1β, IL-6, IL-12, IL-23). As shown at block 226, these inflammatory cytokines can induce the generation and expansion of inflammatory Th1 and Th17 lymphocytes, which can play an important pathogenic role in the progression of DED.

### THO-Dependent Activation of T Cell-Driven Detrimental Immune Responses in the Eyes of DED Patients

THO-dependent apoptosis of corneal epithelial cells can disrupt the integrity of the mucosal barrier at the ocular surface, enabling the influx of invading pathogens. Antigens that bypass a THO-injured epithelial barrier can be captured by professional antigen-presenting DCs that reside in the corneal epithelium. THO induces the increased activation and maturation of DCs and promotes their recruitment to inflamed corneas and conjunctivas by, for instance, enhancing the expression of adhesion molecules on epithelial and/or endothelial cells. Accordingly, significantly increased numbers of activated DCs can be observed at the ocular surface in animal models (e.g., in THO mice as compared to healthy controls).

After engulfing invading pathogens, activated DCs process their antigens and present them to naïve CD4+ T lymphocytes in regional lymph nodes. DCs isolated from eye-draining lymph nodes in animal models (e.g., THO mice) may have better antigen-presenting properties than DCs which infiltrated regional lymph nodes of control animals. Further, a higher expression of molecules responsible for the DC-dependent activation of naïve T cells (e.g., co-stimulatory and major histocompatibility complex (MHC) proteins) may be observed on the membranes of THO-primed corneal and conjunctival DCs, indicating that a hyperosmolar microenvironment can promote efficient antigen presentation and optimal activation of T cells. Accordingly, higher number of activated CD4+ T cells may be observed in eye-draining lymph nodes in animal models (e.g., in THO mice as compared to control mice).

In corneal and conjunctival DCs, THO can promote MKK7- and JNK-dependent activation of c-Jun and activating transcription factor 2 (ATF2), which results in enhanced production of various inflammatory cytokines (e.g., IL-12, IL-1β, IL-6, and IL-23). While DC-derived IL-12 can trigger Th1-driven eye inflammation, DC-sourced IL-1β, IL-6, and/or IL-23 may be responsible for the generation of Th17 cell-dependent injury of meibomian and lacrimal glands. Through the production of IL-12, DCs can activate Signal Transducer and Activator of Transcription (STAT)-1 and T-bet transcriptional factors in naïve CD4+ T lymphocytes, inducing their differentiation in effector, IFN-producing Th1 cells. Similarly, by activating STAT-3 and RAR-related orphan receptor gamma T (RORyT) transcriptional factors in naïve CD4+ T cells, DC-derived IL-1β, IL-6, and/or IL-23 can induce the generation and expansion of inflammatory CD4+ Th17 cells.

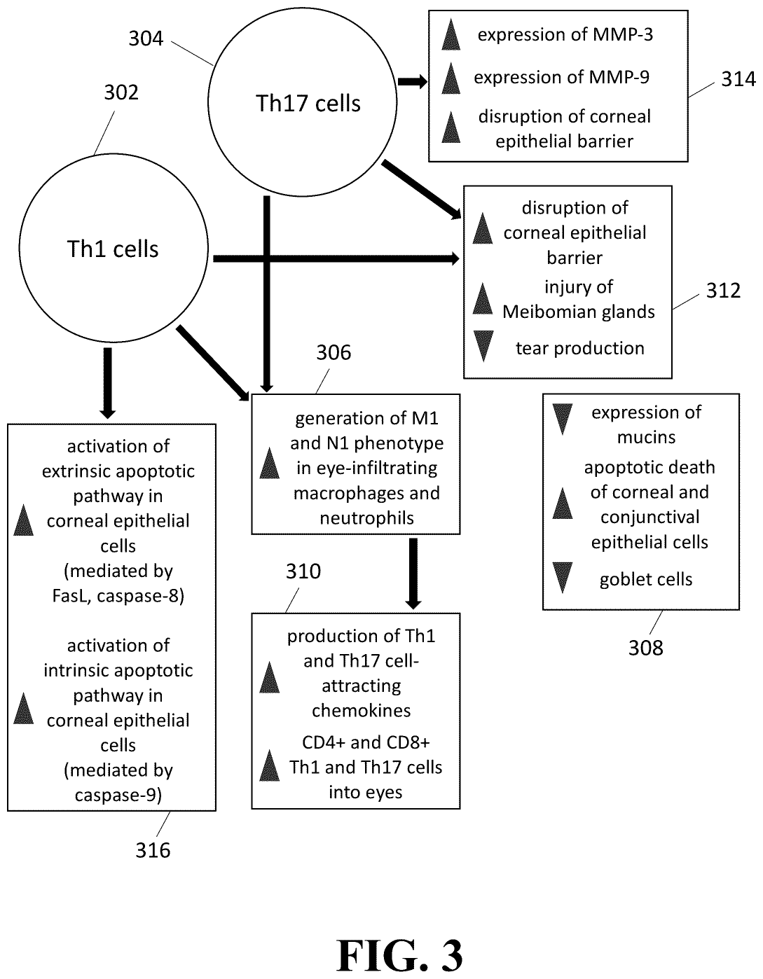

Turning now to FIG. 3, various non-limiting effects of Th1 cells (block 302) and Th17 cells (block 304) are shown. CD4+ Th1 lymphocytes, in an IFN-γ dependent manner, and CD4+ Th17 lymphocytes, in an IL-17 and IL-22-dependent manner, can induce the generation of the inflammatory M1 and N1 phenotype in eye-infiltrating macrophages and neutrophils of DED patients, as shown at block 306. These cells are a valuable source of inflammatory mediators, which contribute to various effects, as shown at block 308, including: (1) reduced expression of mucins, (2) apoptotic death of corneal and conjunctival epithelial cells, and/or (3) loss of goblet cells in inflamed eyes of DED patients. Additionally, M1 macrophages and N1 neutrophils produce Th1 and Th17 cell-attracting chemokines (e.g., C—X—C motif ligand (CXCL)-9/-10, C—C motif ligand (CCL)-20), enabling a large influx of CD4+ and CD8+ Th1 and Th17 cells into the inflamed eyes of DED patients, shown at block 310.

Th1 and Th17 lymphocytes, through the production of inflammatory cytokines (e.g., IFN-γ and IL-17) can also produce a variety of effects in animal models (e.g., in the eyes of DED mice). Non-limiting examples of these effects are shown at block 312 and include: (1) inducing disruption of the corneal epithelial barrier, (2) triggering injury of meibomian glands, and/or (3) reducing tear production. Antibody-dependent neutralization or genetic deletion of IFN-γ and IL-17 can significantly attenuate eye inflammation and can prevent corneal epithelial barrier dysfunction in the eyes of DED mice. This indicates an important pathogenic role for these inflammatory cytokines in the pathogenesis of DED. Th17 cell-derived IL-17 can increase the expression of MMP-3 and MMP-9 which, in turn, induces disruption of the corneal epithelial barrier, shown at block 314.

Th1 cell-sourced IFN-γ can activate both extrinsic and intrinsic apoptotic pathways in the corneal epithelial cells, as shown at block 316. The extrinsic or “death receptor” route is mediated by Fas ligand (FasL) and TNF-related apoptosis-inducing ligand (TRAIL)-dependent activation of caspase-8, and the intrinsic or “mitochondrial” route is mediated by caspase-9. While exogenous administration of IFN-γ may significantly increase caspase-8 and -9-dependent apoptosis of conjunctival epithelial cells, pharmacological inhibition or genetic deletion of IFN-γ can result in the remarkable alleviation of immune cell driven injury of conjunctival epithelial and goblet cells in the eyes of animal models (e.g., DED mice). Similar findings may also hold true for DED patients. Further, IL-17-dependent corneal barrier disturbance may be linked to an increased risk of various complications, including, but not limited to, corneal ulcers, corneal haze, and vision loss. Increased levels of IFN-γ may be linked to conjunctival epithelial squamous metaplasia with progressive goblet cell loss.

Passive transfer experiments can be used to investigate whether detrimental Th1/Th17 cell-driven immune responses, initiated by the disruptive effect of THO, are sufficient to induce DED under various conditions (e.g., mild desiccating stress). Animal models, such as mice, can be divided into different experimental groups, receiving T cells from either THO or control (healthy) animals. In the non-limiting case of mice, the T cells may be taken from, for instance, the spleens and lymph nodes. Passive transfer of T lymphocytes can then be achieved, followed by exposure of the animals to the aforementioned specific conditions (e.g., mild desiccating stress). Such mild desiccating stress can be achieved via, for instance, forced airflow without scopolamine. Under such experimental conditions, it is possible that CD4+ and CD8+ T cells from THO mice can home more efficiently to lymph nodes than their control counterparts. They may also induce strong and potent detrimental immune responses that can cause DED-related clinical signs in recipient animals. The eyes of experimental animals that received T cells from THO mice may exhibit, for instance, (1) a gradual reduction in tear production, and/or (2) damage and increased irregularity of the corneal surface. These pathological changes may not be observed in animals that were injected with T cells from control animals, indicating a potentially important role of THO for the priming of T cell-driven eye inflammation.

It is possible that passive transfer of THO-primed T cells manages to induce DED in recipient animals only upon the depletion of immunosuppressive FoxP3-expressing CD4+ T regulatory cells (Tregs). Counter to inflammatory, IFN-γ and IL-17-producing Th1 and Th17 cells, Tregs produce immunomodulatory factors (e.g., retinoic acid, IL-10, transforming growth factor beta (TGF-β)) that (1) attenuate maturation and antigen-presenting properties of ocular DCs, (2) prevent generation of CD4+ Th1 and Th17 lymphocytes in eye-draining lymph nodes, and/or (3) inhibit production of inflammatory cytokines (e.g., TNF-α and IL-1β) and Th1- and Th17-attracting chemokines (e.g., CXCL9, CXCL10, CCL-20) in N1 neutrophils and M1 macrophages. Such effects may importantly contribute to the creation of an anti-inflammatory microenvironment in the eyes of DED patients. THO impairs immune homeostasis at the ocular surface by, for instance, altering the ratio between inflammatory and regulatory T cells. Additionally, THO can favor the generation of a pro-inflammatory (immunogenic) phenotype, instead of an immunosuppressive (tolerogenic) phenotype, in eye-infiltrated DCs. While immunogenic DCs can induce eye inflammation by promoting activation of Th1 and Th17 cells, tolerogenic DCs can create an immunosuppressive microenvironment in inflamed eyes of DED patients by, for instance, inducing expansion of Tregs. In animal models (e.g., THO mice), there may be a significantly reduced presence of immunosuppressive Tregs, and an increased number of activated, CD69-expressing T lymphocytes, memory (CD62L^{lo} CD44^{hi}) CD4+ T cells, and CD103-expressing CD8+ cytotoxic T cells, in the draining lymph nodes of THO mice as compared to healthy controls. This may indicate that the THO-dependent modulation of DCs' phenotype and function can alter the cellular make-up of eye-draining lymph nodes and promote the progression of T cell-driven eye inflammation.

### THO-Dependent Modulation of Eye-Infiltrated Neutrophils and Natural Killer Cells

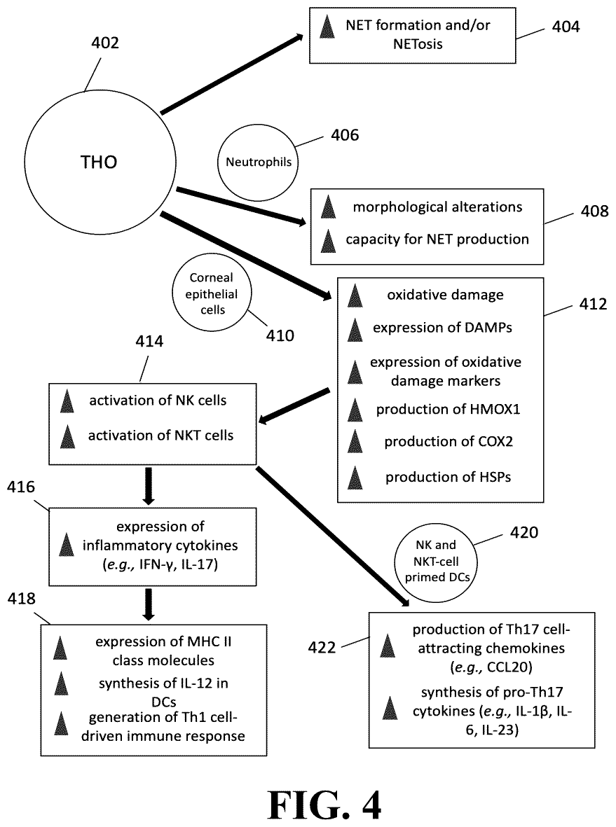

Various lines of evidence have indicated that, in addition to macrophages, DCs, and T cells, THO can modulate the phenotype and/or function of eye-infiltrated neutrophils and natural killer (NK) cells. Non-limiting examples of these are shown in FIG. 4.

Neutrophil extracellular trap (NET) formation and/or NETosis is a process by which neutrophils release their DNA and antimicrobial substances to trap and kill invading pathogens, a process that can occur in the eyes of DED patients. However, excessive NETosis can cause damage to the ocular surface and exacerbate any ongoing inflammation, leading to further tissue damage and a worsening of DED-related symptoms. Indeed, THO, shown at block 402, can promote NET formation and/or NETosis in inflamed eyes, shown at block 404. This can lead to the aggravation of DED. Further, neutrophils 406 exposed to hyperosmolar conditions can have various morphological alterations (e.g., bigger and more rounded nuclei) and an increased capacity for NET production, shown at block 408. Indeed, if isoosmolar conditions are restored, it is possible for NET formation to be significantly reduced. Furthermore, the inclusion of NET formation inhibitors (e.g., staurosporine, anti-2 integrin) can result in a reduction in the quantity of NETs under hyperosmolar conditions.

Since they are an early source of IFN-γ during the induction phase of experimental dry eye illness, natural killer (NK) and natural killer T (NKT) cells have been implicated in the immunopathogenesis of DED. In corneal epithelial cells 410, THO can induce oxidative damage, which can manifest in an increased expression of damage associated molecular patters (DAMPs) and oxidative damage markers, and with an increased production of heme oxygenase-1 (HMOX1), cyclooxygenase-2 (COX2), and/or one or more heat shock proteins (HSPs), all shown at block 412. NK and NKT cells can recognize DAMPs and oxidative stress-related proteins that are expressed on the membranes of THO-exposed corneal epithelial cells. This recognition can trigger the activation of NK and NKT cells, shown at block 414. Such activation can result in an increased production of one or more inflammatory cytokines, shown at block 416. These cytokines include, for instance, IFN-γ and IL-17. NK and NKT cell-sourced IFN-γ may enhance the expression of major histocompatibility complex (MHC) class II molecules and promote the synthesis of IL-12 in DCs, enabling the generation of a Th1 cell-driven immune response in the eyes of DED patients, all shown at block 418. Similarly, NK and NKT-cell primed DCs 420 can exhibit an increased production of Th17 cell-attracting chemokines (e.g., CCL20) and an enhanced synthesis of pro-Th17 cytokines (e.g., IL-1β, IL-6, and IL-23), shown at block 422. Alleviation of THO, as well as depletion of NK and/or NKT cells, can cause one or more of the following effects in DED animals (e.g., DED mice): (1) a significant reduction in the presence of activated DCs in the inflamed eyes of such animals, (2) an alleviation of their antigen-presenting properties, (3) an attenuation of Th1 and/or Th17 cell-driven eye inflammation, (4) downregulation of MMP-3 and/or MMP-9 expression, and (5) reduction in apoptosis of corneal and conjunctival epithelial cells. Such effects confirm the important pathogenic role of THO for NK and NKT cell-driven inflammation in the eyes of DED patients.

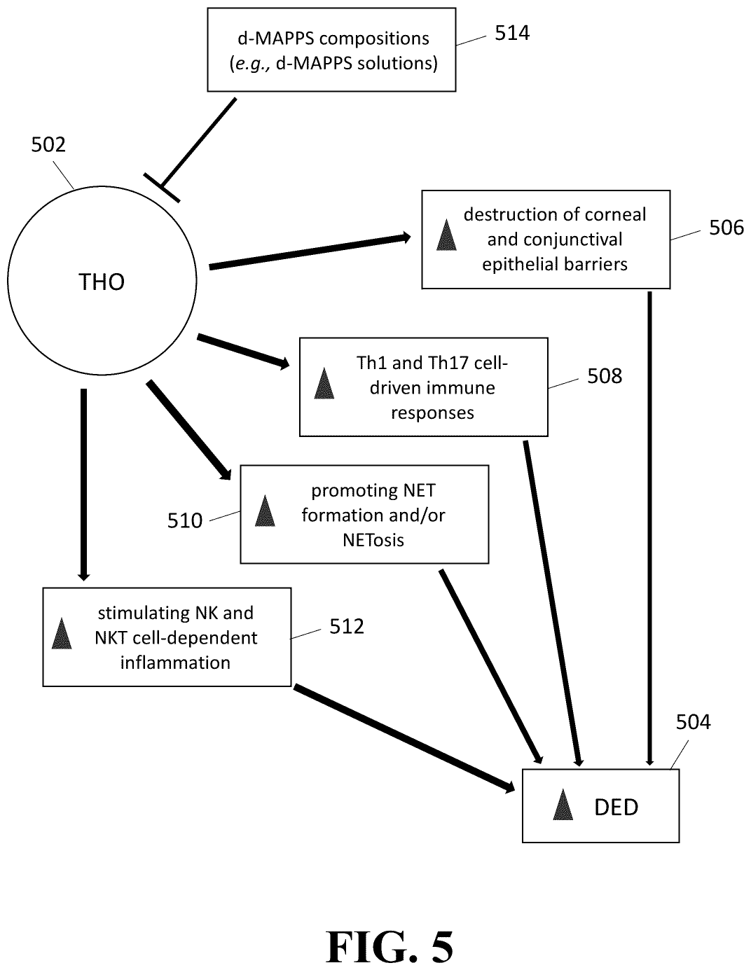

Thus, the above indicates that THO initiates and/or aggravates DED by various processes, non-limiting examples of which are shown in FIG. 5. THO, shown at block 502, can initiate and/or aggravate DED, shown at block 504, by one or more of: (1) inducing the destruction of corneal and conjunctival epithelial barriers, shown at block 506, (2) enhancing Th1 and Th17 cell-driven immune responses, shown at block 508, (3) promoting NET formation and/or NETosis, shown at block 510, and (4) stimulating NK and NKT cell-dependent inflammation (e.g., in the inflamed eyes of DED patients), shown at block 512.

Given that THO plays an important pathogenic role in the development and/or progression of DED, eye drops, alleviation and/or treatment of THO can be achieved using the d-MAPPS compositions (including, for instance, one or more d-MAPPS solutions) described herein, shown at block 514. These compositions can be administered via topical administration of eye-drops (e.g., one or more d-MAPPS solutions described herein) that are designed specifically to address THO and/or DED.

Treatment of Dry Eye and/or Tear Hyperosmolarity Via d-MAPPS Compositions and/or d-MAPPS Solutions

In at least one embodiment, d-MAPPS compositions, including, but not limited to, d-MAPPS solutions, treat DED and/or tear hyperosmolarity. The d-MAPPS compositions may be, for instance, hypoosmotic in nature (e.g., hypoosmotic eye drops). Specifically, the compositions may contain a lower concentration of salt and/or other solutes compared to the tears on the ocular surface, which can help to reduce THO and improve one or more DED symptoms (e.g., dryness, burning, and eye irritation). Without wishing to be bound by theory, the d-MAPPS compositions described herein can, when administered into the eyes of DED patients, provide one or more benefits, including, for instance: (1) helping to restore the natural osmotic balance of the ocular surface, (2) improving tear film function, (3) decreasing inflammation, and (4) decreasing THO-induced damage of the corneal and/or conjunctival epithelial barriers.

In at least one example, one or more d-MAPPS compositions comprise one or more anti-inflammatory agents, one or more immunosuppressive drugs, and/or one or more immunomodulatory drugs. Such agents and/or drugs may further manage and/or treat one or more aspects of DE and/or DED (e.g., managing inflammation, improving DED symptoms, etc.). For instance, since THO can induce apoptosis of the corneal and/or conjunctival epithelial cells by triggering activation of NF-κB signaling pathways in corneal epithelial cells, combined use of NF-κB inhibitors and hypoosmotic ophthalmic solutions (e.g., one or more d-MAPPS compositions described herein) could be used to abrogate DED-related signs and symptoms in the injured and/or inflamed eyes of DED patients. In at least one example, one or more d-MAPPS compositions themselves comprise one or more NF-κB inhibitors.

In at least a further example, one or more d-MAPPS compositions comprise one or more osmoprotectants, which are naturally occurring compatible solutes (e.g., natural water) that can be internalized by cells. This results in a restoration of cell volume and stabilizing proteins. The osmoprotective effect depends on the quantity of osmoprotectants taken up by the cell and the length of retention. For example, glycerol can rapidly and easily enter the cell via the water channel, but also leaves the cells very quickly. Therefore, glycerol's therapeutic effects could be improved if combined with a protectant that acts over a longer period of time.

It should be appreciated that one or more of the d-MAPPS compositions described herein may be in solution form (e.g., the d-MAPPS solutions described herein), and further that such solutions need not be biologics, but can be over-the-counter (OTC) drug products manufactured under current Good Manufacturing Practices (cGMP) and regulated by the Food and Drug Administration (FDA). Accordingly, the d-MAPPS solutions provide safe and effective eye-drops that are immediately ready for clinical use.

### Formulations

In at least one example, one or more d-MAPPS solutions is a bio-engineered biologic product obtained from one or more types of MSCs, such as, for instance, amniotic fluid derived MSCs (AF-MSCs). Such MSCs are collected from healthy human donors. The human donors are tested for various diseases via blood samples given by the donor prior to, or at the time of, collection. Testing is done using United States (U.S.) Food and Drug Administration (FDA) licensed tests for detection of at minimum: Hepatitis B Virus, Hepatitis C Virus, Human Immunodeficiency Virus Types 1/2, and Treponema Pallidum. AF samples are obtained with patient consent and kept at 4° C. until processed. The one or more d-MAPPS solutions are then bio-engineered as an AF-MSC-derived sterile product containing, among other components, AF-MSC-Exos and AF-MSC-derived cytokines and growth factors. The one or more d-MAPPS solutions are manufactured under current Good Manufacturing Practices (cGMP), regulated and reviewed by the FDA. The one or more d-MAPPS solutions are then sterilized to ensure a safe, sterile product.

In at least one example, the one or more d-MAPPS solutions are hypotonic solutions containing one or more osmoprotectants that address and/or remedy the hyperosmolarity of tear film. The one or more osmoprotectants may include a variety of compounds, such as natural water (e.g., undiluted and/or pure water), one or more sugars (e.g., sucrose, trehalose, gentiobiose, melibiose, maltose, turanose, raffinose, stachyose, verbascose, altrose, palatinose, cellobiose) and/or their derivatives, one or more amino acids (e.g., glutamine, proline, alanine, carnitine) and/or their derivatives, one or more polyols (e.g., glycerol, arabitol, inositol, mannitol, sorbitol, maltitol) and/or their derivatives, one or more heterosides (e.g., glucosylglycerol, mannosucrose) and/or their derivatives, glycine betaine, and/or trimethylglycine. The one or more osmoprotectants may also be, for instance, one or more naturally occurring solutes that are compatible with, and can be internalized by, cells (e.g., one or more cells of the eye and/or glands associated with the eye).

Accordingly, one or more tonicity adjusting agents may be added to provide the desired ionic strength and/or to ensure the d-MAPPS solutions are hypotonic. Tonicity-adjusting agents for use include those which display no or only negligible pharmacological activity after administration. Both inorganic and organic tonicity adjusting agents may be used. The d-MAPPS solutions can also include excipients and/or additives. Examples of excipients are surfactants, stabilizers, complexing agents, antioxidants, or preservatives which prolong the duration of use of the d-MAPPS solutions, flavorings, vitamins, or other additives known in the art. Complexing agents include, but are not limited to, ethylenediaminetetraacetic acid (EDTA) or a salt thereof, such as the disodium salt, citric acid, nitrilotriacetic acid and the salts thereof. Preservatives include, but are not limited to, those that protect the solution from contamination with pathogenic particles, including benzalkonium chloride or benzoic acid, or benzoates such as sodium benzoate. Antioxidants include, but are not limited to, vitamins, provitamins, ascorbic acid, vitamin E or salts or esters thereof.

In at least one example, the one or more d-MAPPS solutions include one or more pharmaceutically acceptable salts, such as, for instance, one or more chloride, acetate, and/or citrate salts. Further non-limiting examples of pharmaceutically acceptable salts include, for example, sodium chloride, potassium chloride, calcium chloride (e.g., calcium chloride dihydrate), magnesium chloride (e.g., magnesium chloride hexahydrate), sodium acetate (e.g., sodium acetate trihydrate), and/or sodium citrate (e.g., sodium citrate dihydrate).

In at least one example, the one or more d-MAPPS solutions include one or more physiological buffers, such as a phosphate (e.g., monobasic sodium phosphate, disodium hydrogen phosphate, potassium dihydrogen phosphate).

In at least one example, the one or more d-MAPPS solutions include hyaluronic acid (e.g., crosslinked hyaluronic acid), sodium hyaluronate, chondroitin sulfate, dermatan sulfate, heparin sulfate, keratin sulfate, hydroxylpropylmethylcellulose, recombinant human collagen, and combinations thereof.

In at least one example, the one or more d-MAPPS solutions include one or more stabilizers, which can maintain and/or improve the physical and/or chemical stability of the solutions. The one or more stabilizers may be hydrated in an aqueous solvent. Non-limiting examples of stabilizers include carboxymethylcellulose, hydroxypropylmethyl cellulose, cellulose-based compounds (e.g., hydroxyethyl cellulose), polyvinyl-based compounds (e.g., polyvinyl alcohol, polyvinylpyrrolidone), acrylic compounds (e.g., one or more carbomers), gum compounds (e.g., gellan gum, xanthan gum), and polysaccharides (e.g., hyaluronic acid, sodium hyaluronate, sodium alginate, dextran).

In at least one example, the one or more d-MAPPS solutions include ophthalmically acceptable demulcents, ophthalmically acceptable excipients/emollients, ophthalmically acceptable astringents, and/or ophthalmically acceptable vasoconstrictors. Non-limiting examples of ophthalmically acceptable demulcents include, for instance, carboxymethylcellulose sodium, hydroxyethyl cellulose, hypromellose, methylcellulose, dextran 70, gelatin, glycerin, polyethylene glycol 300, polyethylene glycol 400, polysorbate 80, propylene glycol, polyvinyl alcohol, and povidone. Non-limiting examples of ophthalmically acceptable excipients/emollients include, for instance, anhydrous lanolin, lanolin, light mineral oil, mineral oil, paraffin, petrolatum, white ointment, white petrolatum, white wax, and yellow wax. Non-limiting examples of ophthalmically acceptable astringents include, for instance, zinc sulfate. Non-limiting examples of ophthalmically acceptable vasoconstrictors include, for instance, ephedrine hydrochloride, naphazoline hydrochloride, phenylephrine hydrochloride, and tetrahydrozoline hydrochloride.

In at least one example, the one or more d-MAPPS solutions may take the form of a solution (e.g., a liquid solution, an aqueous solution), a gel (e.g., a viscoelastic gel), and/or a film (e.g., a viscoelastic film).

In at least one example, the one or more d-MAPPS solutions may be balanced and/or buffered. The pH may be, for instance, a physiological pH such as, for instance, about 6.0 to about 8.0, or at any pH and/or pH range therebetween (e.g., pH 7.0). Thus, the pH of the one or more d-MAPPS solutions may be, for example, about 6.5 to about 7.8, about 6.5 to about 7.5, about 6.8 to about 7.6, about 7.0 to about 7.4, about 7.0 to about 7.2, or about 6.8 to about 7.2. The one or more d-MAPPS solutions may further include one or more balanced salt solutions. Non-limiting examples of salts that can be incorporated into the one or more balanced salt solutions include, for instance, sodium chloride, potassium chloride, calcium chloride dehydrate, magnesium chloride hexahydrate, sodium acetate trihydrate, and/or sodium citrate dihydrate. The pH of the balanced salt solution may have a pH of about 6.0 to about 8.0, and the balanced salt solution may include sodium hydroxide and/or hydrochloric acid to adjust pH.

In at least one example, the one or more d-MAPPS solutions may have an osmolality ranging from about 200 mOsm/kg to about 400 mOsm/kg, such as, for instance, 300 mOsm/kg. The one or more d-MAPPS solutions may include one or more buffer solutions (e.g., an infusion buffer solutions). Such buffer solutions may be, for instance, a phosphate buffer solution, a buffer solution containing sodium chloride, and/or a buffer solution containing potassium chloride. Further suitable physiological buffers may be used. The buffer solutions may have a concentration ranging from, for instance, about 0.005 M to about 1.0 M, about 0.01 M to about 1.0 M, about 0.01 M to about 0.5 M, about 0.05 M to about 1.0 M, about 0.1 M to about 0.5 M, or about 0.5 M to about 1.0 M.

In at least one example, the one or more d-MAPPS solutions have a viscosity ranging from about 50,000 milliPascal-seconds (mPa sec) to about 160,000 mPa sec, about 50,000 mPa sec to about 75,000 mPa sec, about 50,000 mPa sec to about 55,000 mPa sec, about 90,000 mPa sec to about 110,000 mPa sec, about 100,000 mPa sec to about 150,000 mPa sec, about 125,000 mPa sec to about 150,000 mPa sec, about 130,000 mPa sec to about 140,000 mPa sec, or about 120,000 mPa sec to about 140,000 mPa sec.

In at least one example, the concentration of one or more MSCs, one or more MSC-Exos, and/or one or more MSC-Exos-derived proteins and/or compounds (e.g., growth factors, cytokines) ranges from about 0.001 mg/mL to about 10 mg/mL, about 0.001 mg/mL to about 1.0 mg/mL, about 0.005 mg/mL to about 0.1 mg/mL, about 0.01 mg/mL to about 1.0 mg/mL, about 0.05 mg/mL to about 2.0 mg/mL, about 0.07 mg/mL to about 2.5 mg/mL, about 0.1 mg/mL to about 3 mg/mL, about 0.5 mg/mL to about 1 mg/mL, about 1 mg/mL to about 6 mg/mL, about 1 mg/mL to about 5 mg/mL, about 1.5 mg/mL to about 4.5 mg/mL, about 2 mg/mL to about 4 mg/mL, about 3 mg/mL to about 5 mg/mL, about 4 mg/mL to about 5 mg/mL, about 2 mg/mL to about 4 mg/mL, about 0.5 mg/mL to about 2.5 mg/mL, about 1 mg/mL to about 2 mg/mL, about 3.5 mg/mL to about 5 mg/mL, or about 5 mg/mL to about 10 mg/mL.

In at least one example, the concentration of one or more MSCs, one or more MSC-Exos, and/or one or more MSC-Exos-derived proteins and/or compounds (e.g., growth factors, cytokines) ranges from about 10 μg/mL to about 500 μg/mL, about 50 μg/mL to about 250 μg/mL, about 100 μg/mL to about 500 μg/mL, about 150 μg/mL to about 300 μg/mL, or about 250 μg/mL to about 500 μg/mL.

In at least one example, the total volume of one or more aliquots, samples, and/or doses of the one or more d-MAPPS solutions ranges from about 0.050 mL to about 2.0 mL, such as, for example, about 1.0 mL. Thus, the amount of one or more d-MAPPS solutions administered to a patient's eye at any one given time may range from about 0.050 mL to about 2.0 mL, such as, for example, about 1.0 mL.

Thus, in at least one example, the dose of one or more MSCs, one or more MSC-Exos, and/or one or more MSC-Exos-derived proteins and/or compounds (e.g., growth factors, cytokines) administered to a patient's eye ranges from about 0.2 mg/100 μL (2 mg/mL) to about 0.6 mg/100 μL (2 mg/mL) per treatment, about 0.25 mg/100 μL to about 0.5 mg/100 μL, about 0.25 mg/100 μL to about 0.55 mg/100 μL, about 0.28 mg/100 μL to about 0.475 mg/100 μL, about 0.3 mg/100 μL to about 0.5 mg/100 μL, about 0.35 mg/100 μL to about 0.45 mg/100 μL, about 0.25 mg/100 μL to about 0.5 mg/100 μL, about 0.45 mg/100 μL to about 0.5 mg/100 μL, or about 0.3 mg/100 μL to about 0.4 mg/100 μL. Further non-limiting examples of dosages include, for instance, from about 0.005 mg/mL to about 2.0 mg/mL, about 0.025 mg/mL to about 1.0 mg/mL, or about 0.05 mg/mL to about 0.2 mg/mL.

In at least one example, the dose of one or more MSCs, one or more MSC-Exos, and/or one or more MSC-Exos-derived proteins and/or compounds (e.g., growth factors, cytokines) administered to a patient's eye ranges from about 0.005 mg (5 μg) to about 2.0 mg per total delivery volume of the one or more d-MAPPS solutions, about 0.01 mg to about 2 mg, about 0.05 mg to about 0.5 mg, about 0.1 mg to about 1.0 mg, about 0.5 mg to about 1.8 mg, about 0.75 mg to about 1.5 mg, about 0.5 mg to about 1.25 mg, about 0.75 mg to about 1 mg, about 0.5 mg to about 0.75 mg, about 0.25 mg to about 0.75 mg, about 0.6 mg to about 1.2 mg, about 0.9 mg to about 1.3 mg, or about 1.5 mg to about 1.8 mg per total delivery volume of the one or more d-MAPPS solutions. As non-limiting examples, the total delivery volume of the one or more d-MAPPS solutions may range from about 0.1 mL to about 2.0 mL, about 0.1 mL to about 0.5 mL, about 0.2 mL to about 0.35 mL, 0.25 mL to about 0.75 mL, about 0.25 mL to about 0.45 mL, about 0.5 mL to about 1.0 mL, about 0.5 mL to about 2.0 mL, about 1.0 mL to about 2.0 mL, or about 1.0 mL to about 1.5 mL per application (e.g., one or more applications) or per eye (e.g., administered in one or more applications). For instance, the volume of the one or more d-MAPPS solutions administered may be less than 1 mL, such as, for instance, in a range of about 0.1 mL to about 0.9 mL.

### Storage

In at least one embodiment, the one or more d-MAPPS solutions can be stored in frozen conditions at about −20° C. to about −80° C. In addition, the d-MAPPS solutions may be distributed in vials equipped with special rubber stoppers for sterile lyophilization. Lyophilization is generally carried out in a sterile environment. The rubber stoppers on the vials are then automatically pushed down in the freeze dryer to definitively close them. Then, an aluminum cap is sealed on each vial to protect its sterile content. In such a lyophilized state, the d-MAPPS solutions may be stored at +4° C. or room temperature for at least one year without decrease of one or more components thereof, such as, for example, one or more MSCs, one or more MSC-Exos, and/or one or more MSC-Exos-derived proteins and/or compounds (e.g., growth factors, cytokines). Before use, lyophilized d-MAPPS solutions may be reconstituted by adding the initial volume of sterile water to the powder in order to restore a transparent and homogeneous physiological liquid.

In at least one example, the one or more d-MAPPS solutions contain growth factors and other biological components that are stabilized against degradation (e.g., chemical and/or enzymatic degradation). Molecules contained within the fluid are stabilized against degradation, avoiding the need for chemical or physical modification to maintain the biological activity of the molecules over extended periods of time. Therefore, the one or more d-MAPPS solutions can be stored and/or distributed for long periods of time, allowing for a broad range of application and/or treatment methods.

In at least one example, the one or more d-MAPPS solutions can be stored in refrigerated conditions at about 1° C. to about 10° C. For instance, the d-MAPPS solutions can be refrigerated at 4° C. for up to 12 months and more.

In at least one example, the one or more d-MAPPS solutions can be stored at room temperature for over a week, 2 weeks, 3 weeks, a month, 2 months, 3 months, 6 months, or up to 12 months or more, while still retaining most biologically active components such as, for example, one or more MSCs, one or more MSC-Exos, and/or one or more MSC-Exos-derived proteins and/or compounds (e.g., growth factors, cytokines). The biological activity of such room temperature-stored d-MAPPS solutions is preferably comparable to that of d-MAPPS solutions refrigerated at about 1° C. to about 10° C. and/or d-MAPPS solutions stored at about −20° C. to about −80° C. For example, fluids purified according to the described methods retain the biological properties of the component molecules over extended periods of storage, ideally without the need for freeze/thawing.

In at least one example, storage of the one or more d-MAPPS solutions, at any temperature and/or temperature range described herein, does not reduce the quantity and/or biological activity of one or more MSCs, one or more MSC-Exos, and/or one or more MSC-Exos-derived proteins and/or compounds (e.g., growth factors, cytokines). Therefore, in at least one example, little or no statistically significant changes in biological activity are observed when storing the one or more d-MAPPS solutions at 4° C. or at room temperature for up to a day, 2 days, 3 days, 4 days, 5 days, 6 days, up to one week, up to 2 weeks, up to 3 weeks, up to 4 weeks, up to one month, up to 2 months, up to 3 months, up to 4 months, up to 5 months, up to 6 months, or more than 6 months.

In at least one example, the one or more d-MAPPS solutions are stored, without degradation, in any of the storage conditions described herein for at least about 1 day, at least about 2 days, at least about 3 days, at least about 5 days, at least about 1 week, at least about 2 weeks, at least about 3 weeks, at least about 1 month, at least about 2 months, at least about 3 months, at least about 4 months, at least about 5 months, at least about 6 months, at least about 12 months, at least about 18 months, at least about 24 months, at least about 36 months, at least about 3 years, at least about 4 years, or at least about 5 years. During such storage times, degradation of one or more components of the d-MAPPS solutions (e.g., one or more MSCs, one or more MSC-Exos, and/or one or more MSC-Exos-derived proteins and/or compounds) is less than about 30%, less than about 25%, less than about 20%, less than about 15%, less than about 10%, less than about 5%, less than about 3%, less than about 2%, or less than about 1%.

### Administration

In at least one example, the one or more d-MAPPS solutions may be applied to any portion of the eye and/or any bodily structure associated with the eye, including, for instance, the eye itself, the cornea, endothelial tissue, anterior chamber segment tissue, the posterior chamber of the eye, the retina, the epithelium, the native corneal epithelium, the epithelial cells, the lacrimal glands, the meibomian glands, and/or the mucin-producing goblet cells.

In at least one example, the one or more d-MAPPS solutions are formulated in a dosage between about 0.1 ml and about 100 ml, inclusive; or between about 0.1 ml and 1 ml, inclusive; or between about 1 ml and about 10 ml, inclusive; or between about 10 ml and about 50 ml, inclusive. In at least a further example, the formulation is combined with any amount of between about between about 0.1 ml and about 100 ml, inclusive; or between about 0.1 ml and 1 ml, inclusive; or between about 1 ml and about 10 ml, inclusive; or between about 10 ml and about 50 ml, inclusive, of sterile water, or saline solution.

In at least one example, the one or more d-MAPPS solutions are packaged into sterile dosage units which can be stored and distributed for use by attending physicians and/or other healthcare professionals. Lyophilized or fluid formulations can be in the form of sterile packaged ampule ready for use. A filled ampoule contains a formulation of one or more of the d-MAPPS solutions. Generally, such solutions are in one or more pharmaceutically acceptable carriers and buffered for human use to a pH of about 3.5-10.0, preferably about pH 6.0-8.0. In at least one example, the formulations of the one or more d-MAPPS solutions are free of preservatives where such preservatives may exert opposite effects to that required by the formulations. Water or saline solution can be used to provide the carrier.

Generally, volumes used herein refer to d-MAPPS solutions at 1× strength without any dilution or concentration. In at least one example, where lyophilized formulations of d-MAPPS solutions are used, these volumes refer to the volume of fluid when the lyophilized powder is reconstituted with the initial volume of sterile water, i.e., 1× strength. The d-MAPPS solutions and/or formulations can be administered in concentrated form, diluted with sterile water, saline or buffer. The formulation may also include additional therapeutic, prophylactic, or diagnostic agents. Said agent(s) may be in-mixed with the formulations or mixed in separate containers to be used in conjunction with the d-MAPPS solutions and/or formulations. The efficacy of administration is determined by physician evaluations, patient self-evaluations, imaging studies, and/or quality of life evaluations.

In at least one example, the one or more d-MAPPS solutions may be administered to one or more eyes of a patient for various periods of time per treatment. As non-limiting examples, the periods of time per treatment may be at least 10 seconds, at least 30 seconds, at least 1 minute, at least 5 minutes, or at least 10 minutes or more. Any given patient and/or their eyes may be treated multiple times per day, such as, for instance, once per day, twice per day, three times per day, five times per day, or more than five times per day. Further, any given patient and/or their eyes may be treated with one or more d-MAPPS solutions over a total period of time lasting at least one day, at least 24 hours, at least 2 days, at least 3 days, at least one week, at least 3 weeks, at least 6 weeks, or at least 12 weeks or more.

Administration of one or more d-MAPPS solutions, as described herein, helps alleviate THO and/or symptoms of THO. Such solutions may also alleviate discomfort, support tear stability, and help relieve dryness in the eyes of DE and/or DED patients. As mentioned above herein, tear hyperosmolarity is a state in which the osmolarity of tears exceeds that of the epithelial cells, leading to reduced cell volume and increased concentration of solutes. The d-MAPPS solutions help to alleviate THO and/or relieve DED by, for instance, increasing hydration to the cornea and restoring homeostasis at the corneal surface.

Generally, homeostasis of the cornea addresses all hyperosmolarity-related issues and may prevent the development of pathological events elicited by exposition of the ocular surface to hyperosmolar tears. In DE and/or DED patients, addressing THO may result in the following beneficial effects: (1) allowing for hypotonic natural tear stability, (2) preventing oxidative stress in the meibomian glands, the epithelial cells, and the and mucin-producing goblet cells, (3) preventing DNA damage and apoptosis of the cells at the ocular surface, (4) preventing hyperosmolarity-induced production of pro-inflammatory cytokines in eye-infiltrated immune cells, (5) attenuating ongoing eye inflammation by, for instance, inhibiting interleukin (IL)-1 and tumor necrosis factor (TNF)-α-driven inflammation in IL-1Ra and sTNFR-dependent manner, (6) suppressing Th1/Th17 cell-dependent detrimental immune response by, for instance, down-regulating hyperosmolarity induced enhanced MHC expression on DCs, (7) enabling DE patients to relieve dryness of the eye, and (8) enabling improvement of visual acuity and better performance of vision-dependent daily activities.

After administration with d-MAPPS solutions, one or more objective measurements (e.g., tear film breakup time (TBUT), tear film thickness) can be taken to determine the effects of such administration. TBUT is preferably less than about 10 seconds or less than about 5 seconds after one or more courses of treatment with one or more d-MAPPS solutions. Generally, tear film thickness can increase as TBUT increases, as noted by Creech J. L., et al., “In vivo tear-film thickness determination and implications for tear film stability,” Curr. Eye Res. 17:1058-66 (1998). Tear film thickness can be used to measure the liquid layer, the lipid layer, and/or a combination of the liquid layer and the lipid layer. Tear film thickness can be measured by a slit lamp and a video camera. This can be done by, for instance, instilling fluorescein dye in the form of an eyedrop and videotaping the tear meniscus in profile. Tear-film breakup can then be videotaped through the ocular port of the slit lamp and evaluated based on a severity scale. Tear film thickness can also be measured by using an optical interferometer (e.g., a wavelength-dependent optical interferometer). A non-limiting example of measuring tear film thickness using such interferometers is provided in King-Smith, P. E., et al., “The Thickness of the Human Precorneal Tear Film: Evidence from Reflection Spectra,” Invest. Ophthalmol. Vis. Sci. 41(11): 3348-59 (2000). Specifically, multiple measurements are taken of an area of a subject's eye, where the area has a predetermined length and width. Such lengths and/or widths may range from about 10 μm, about 20 μm, about 30 μm, about 100 μm, or more than about 100 μm. The area may be located in any suitable area of the eye such as, for example, the apex of the cornea. The measurements may be taken over a window of time such as, for instance, about 10 seconds, about 20 seconds, about 30 seconds, about 40 seconds, about 50 seconds, about 1 minute, or more than about 1 minute. The number of measurements may be, for instance, about 10, about 20, about 30, about 50, or more than about 50. Accordingly, a measurement may be taken every about 10 ms, about 20 ms, about 50 ms, about 100 ms, about 200 ms, about 300 ms, about 400 ms, about 500 ms, or more than about 500 ms. Such tear film thickness measurements may be taken both before and after d-MAPPS solution-based treatment. Before treatment, tear film thickness may be, for example, less than about 2 microns, or less than about 1 micron. After treatment, tear film thickness may be, for example, about 5 microns, about 6 microns, about 8 microns, about 10 microns, about 12 microns, or more than about 12 microns.

Attenuation of DE Pathology Via d-MAPPS Compositions and/or d-MAPPS Solutions

The therapeutic potential of d-MAPPS compositions and/or d-MAPPS solutions, including the d-MAPPS solutions described herein containing osmoprotectants, was confirmed in clinical settings. These solutions helped suppress and/or reverse hyperosmolarity-induced eye pathology by alleviating ocular discomfort and pain in 131 DE patients. Of these patients, 27 were male and 104 were female, with a median age of 62 years (ranging from 19 to 85 years of age). The d-MAPPS solutions remarkably improved all DE-related symptoms including pain, dryness, grittiness, scratchiness, soreness, irritation, burning, watering, and eye fatigue.

DE questionnaires showed a significantly reduced Visual Analogue pain Score (VAS) and Standard Patient Evaluation of Eye Dryness Questionnaire (SPEED) score in patients treated with the d-MAPPS solutions. Further beneficial effects were noticed during the entire observational period. For some patients, these effects significantly increased during the last 6 months of the 12-month follow-up. A significant decrease in VAS and SPEED scores for treated patients was documented 3 months after administration with the d-MAPPS solutions. The highest reduction in VAS and SPEED scores was observed after 12 months of therapy with the d-MAPPS solutions. These results indicate that d-MAPPS solution-based treatment provides long-lasting beneficial effects in alleviating ocular symptoms in DE patients.

Beneficial effects of d-MAPPS solutions in the treatment of MGD-related DE have also been demonstrated. The d-MAPPS solutions efficiently attenuated THO and/or DE-related symptoms in patients suffering from MGD. Before topical application of the d-MAPPS solutions, the meibomian ducts of MGD patients were dilated, and the meibomian glands themselves were enlarged and tortuous with abnormal structure. The morphology of the meibomian glands was significantly improved after 3 weeks of d-MAPPS solution-based therapy, showing hypoilluminescent grape-like clusters. Similarly, hypoilluminescent ducts and the underlying tarsus indicated beneficial effects of the d-MAPPS solutions in restoring meibomian gland and duct morphology. Before topical application of the d-MAPPS solutions, MGD patients reported foreign body sensations and pain in the eyes, which were accompanied by grittiness, soreness, irritation, burning, and eye fatigue. Importantly, none of these DE-related symptoms were reported by MGD patients after 3 weeks of d-MAPPS solution-based therapy. Significantly improved tear film breakup time (TBUT) was noticed 3 weeks after d-MAPPS solution-based treatment, indicating better meibomian gland function.

In DE patients treated with d-MAPPS solutions, significantly improved visual acuity, ocular pain relief, and healing of corneal epithelial defects were noticed. In these patients, DE developed due to various causes, including underlying autoimmune disease and Sjogren's syndrome. For instance, a 26-year-old female suffered from severe DE and epithelial basement membrane dystrophy with recurrent corneal erosion syndrome. For this patient, four weeks of d-MAPPS solution-based therapy resulted in remarkably improved visual acuity and significantly decreased ocular pain. Similarly, 15 days of d-MAPPS solution-based therapy significantly alleviated neurotrophic keratitis in an 80-year old patient. This patient used the d-MAPPS solutions 3-4 times a day for 4 days and 2-3 times a day for 11 days. After treatment, her keratitis was nearly resolved.

### Mesenchymal Stem Cells (MSCs) and MSC-Mediated Tissue Repair and Regeneration

Mesenchymal stem cells (“MSC” or “MSCs”) are self-renewable adult stem cells which are able to differentiate into corneal epithelial cells under specific culture conditions. Additionally, MSC secrete a large number of growth factors that support the viability of injured cells and produce immunomodulatory proteins which can regulate the phenotype and/or function of immune cells that participate in the development and progression of dry eye disease, dry eye discomfort, tear hyperosmolarity, and/or tear hyperosmolarity-induced pathological changes in the eyes of patients suffering from dry eye discomfort. Many MSC-derived bioactive factors are contained in MSC-sourced exosomes (MSC-Exos), extracellular vesicles which, due to their nano-sized dimension and lipid envelope, can easily bypass all biological barriers to reach the target epithelial and/or immune cells in the eyes and lacrimal system of DE and/or DED patients without affecting neighboring parenchymal cells and, therefore, without causing any severe side effects. Due to their enormous differentiation potential and immunosuppressive characteristics, MSC and MSC-Exos are new remedies in regenerative ophthalmology. Accordingly, in at least one embodiment of the present disclosure, d-MAPPS pharmaceutical compositions, including d-MAPPS solutions, include one or more MSCs and/or one or more MSC-Exos, as set forth in further detail herein.

MSC may, under specific culture conditions, differentiate in the cells of all three germ layers. Multi-lineage differentiation potential of MSCs could be a consequence of their complex development origin. During embryogenesis, different subpopulations of MSCs originate from different precursor cells, including epithelial-to-mesenchymal transition-derived cells, Sox1+ neuroepithelial cells, lateral plate mesoderm-derived mesoangioblast cells from the embryonic dorsal aorta, and blood-vessel-derived precursor cells.

MSC reside in almost all postnatal tissues from where MSC can be isolated, propagated in vitro, and used in cell-based therapies of degenerative and inflammatory diseases. For clinical use, MSCs can be most frequently derived from bone marrow (BM), umbilical cord (UC), amniotic fluid (AF), and adipose tissue (AT). Specific functional properties of BM-derived MSC (“BM-MSC” or “BM-MSCs”) which favor their clinical application include, for instance, rapid proliferation in vitro, genomic stability after long-term cultivation, and the capacity for the increased production of immunosuppressive cytokines. Although BM-MSC have enormous therapeutic potential, harvesting of BM is an invasive procedure and, therefore, UC, AF, and AT can be used as alternative tissue sources for the isolation of MSCs. Collection of UC-derived MSC (“UC-MSC” or “UC-MSCs”) is noninvasive, painless, and safe. UC-MSC share similar functional properties with BM-MSC, but have a higher capacity for exosome (Exos) production. AF, obtained through amniocentesis, serves as an important source of AF-derived MSC (“AF-MSC” or “AF-MSCs”). AF-MSCs can produce large amount of neurotrophins and have a high therapeutic potential in the repair and regeneration of injured neural cells. Lastly, AT-derived MSC (“AT-MSC” or “AT-MSCs”), easily derived from patients' AT, are usually used for autologous transplantation of MSC. AT-MSC have a high proliferation capacity and potent immunoregulatory properties.

Under specific culture conditions, BM-MSC and AT-MSC may differentiate into corneal epithelial cells. For instance, one week of exposure to hormonal epidermal medium (SHEM) or standard MSC cultured Dulbecco's Modified Eagle Medium (DMEM) supplemented with all-trans-retinoic acid (ATRA), may be sufficient to cause both BM-MSC and AT-MSC to differentiate into corneal epithelial cells.

Higher expression of epithelial markers (e.g., cytokeratin (CK)12, CK3, CK19, E-cadherin) and lower expression of mesenchymal markers (e.g., Vim, snail and alpha smooth muscle actin (α-SMA)) can occur in BM-MSC and AT-MSC which were cultured in SHEM (MSC^{SHEM}) or ATRA-supplemented medium (MSC^{ATRA}) than in BM-MSC and AT-MSC that grew under standard culture conditions (MSC^{DMEM}). Down-regulation or suppression of the Wnt/β-catenin signaling pathway is crucially responsible for BM-MSC and AT-MSC differentiation towards corneal epithelial cells. Importantly, human corneal epithelial cells (HCE) that were co-cultured with MSC^{SHEM} or MSC^{ATRA} can have an increased proliferation rate and an improved capacity for wound healing than HCE which grew with MSC^{DMEM}. The fact that MSC^{SHEM} or MSC^{ATRA} may better guide HCE-driven wound healing than MSC^{DMEM} indicates that SHEM or ATRA not only increases expression of pro-epithelial genes in MSC, but can also induce enhanced secretion of MSC-derived bioactive factors, which improve the viability and proliferation rate of injured HCE. From 720 different proteins which were detected in BM-MSC and AT-MSC-sourced secretome, around 122 proteins participate in the proliferation and differentiation of corneal epithelial cells. Specific proteins such as, for instance, TGF-β receptor type-1, TGF-β receptor type-2, Ras-related C3 botulinum toxin substrate 1, and/or Ras-related C3 botulinum toxin substrate 2 derived from UC-MSC can be responsible for MSC-mediated regulation of epithelial cell proliferation. These molecules activate Jun-N-terminal kinase (JNK) and p38 mitogen activated kinase in HCE, which can elicit signaling pathways that improve their proliferation and migration, which may contribute to the enhanced healing of corneal wounds.

MSC also may have a capacity to repair and regenerate injured corneal epithelium, meibomian and lacrimal glands, indicating their therapeutic potential in the treatment of dry eye disease, dry eye discomfort, tear hyperosmolarity, and/or tear hyperosmolarity-induced pathological changes in the eyes of patients suffering from dry eye discomfort.

For instance, in a rabbit model of alkaline-induced corneal injury, human BM-MSC can differentiate into corneal epithelial cells and migrate into damaged corneal stroma, which can cause improved survival of corneal stromal cells, resulting in corneal regeneration and attenuation of alkaline-induced corneal damage. AT-MSC may also have therapeutic potential in the treatment of LSCD. For instance, human AT-MSC cultured on fibrin gel and grafted onto the damaged corneal surface of mice may cause the re-population of limbal stem cells and the regeneration of injured corneal epithelium. The effectiveness of MSC in LSCD treatment has also been shown in clinical settings. BM-MSC can be successfully engrafted in the eyes of patients suffering from LSCD, significantly improving corneal epithelial failure.

Additionally, rat BM-MSC has therapeutic potential in the regeneration of meibomian glands and in the restoration of meibomian gland function, at least in the context of benzalkonium chloride (BAC)-induced eye injury in rats as an animal model of DED. Reduction in microvilli at apical portions of corneal epithelium, vascular congestion in meiboian glands, large number of apoptotic cells, decreased number of goblet cells, reduced presence of secretory granules and massive leukocyte infiltration can occur in the eyes of BAC+ saline-treated rats. Topically applied MSC can engraft into the injured meibomian glands and in the damaged conjunctival epithelium, resulting in suppressed detrimental immune response and induced repair and regeneration of injured tissue. Meibomian glands may therefore have normal architecture, a significantly increased number of goblet cells with numerous secretory granules, only a paucity of lymphocytes and neutrophils, and few apoptotic cells were detected in the corneas, conjuctivas, and meibomian glands of BAC+ BM-MSC-treated rats. Additionally, mean aqueous tear volume can significantly increase one week after MSC application, suggesting therapeutic efficacy of BM-MSC in the treatment of MGD and DED.

Murine models of aqueous-deficient dry eye disease (ADDED) have shown that, in addition to the restoration of meibomian gland structure, murine lacrimal gland-derived MSC (“LG-MSC” or “LG-MSCs”) can manage to efficiently regenerate injured lacrimal glands as well. ADDED can be induced by the ligation of the lacrimal duct. Duct ligation can then be removed (e.g., three days later), and MSC or saline can be injected into the lacrimal gland. Duct ligation can induce interstitial edema and massive injury of lacrimal glands. Consequently, acinar cells, which produce and secrete the primary tear fluid, may be shrunken and dysfunctional in ADDED mice. Immediately after their injection, LG-MSC can engraft in the stroma of lacrimal glands, adjacent to acinar structures. Weeks after removal of duct ligation, LG-MSC can recover vital acinar structures to, e.g., 62% of total lacrimal gland tissue, which is an increase of, e.g., 25% compared to spontaneous regeneration after saline injection. Tightly arranged acini, organized in lobules and surrounded by connective tissue, may be observed in MSC-treated lacimal glands, but not in saline-treated lacrimal glands. A higher presence of proliferating, Ki67-positive cells and enhanced expression of MIST1 expression (acinus specific transcription factor) can also be observed in the LG-MSC-treated lacrimal glands, confirming LG-MSC-mediated restoration of acinar cells. Further, significantly reduced expression of caspase-3 in LG-MSC-treated lacrimal glands can indicate that LG-MSC suppressed the apoptosis of acinar cells. As a result of LG-MSC-mediated regeneration of lacrimal glands, the amounts of secreted tears in the eyes of MSC-treated ADDED animals may be, 21 days after MSC injection, similar to the baseline value which were measured at the ocular surface of healthy animals. MSC-dependent suppression of detrimental immune response may also be responsible for the beneficial effects of LG-MSC in the repair and regeneration of lacrimal glands. A significantly reduced number of Ly6G-expressing neutrophils and a lower number of CD68-expressing macrophages may be observed in MSC-treated lacrimal glands, 21 days after LG-MSC transplantation. Additionally, LG-MSC can down-regulate the synthesis of TNF-α in lacrimal gland-infiltrated immune cells and suppress TNF-α-driven injury of acinar cells, significantly contributing to improved tear secretion, suggesting that the immunomodulatory potential of MSC can be important for these beneficial effects in the treatment of dry eye disease, dry eye discomfort, tear hyperosmolarity, and/or tear hyperosmolarity-induced pathological changes in the eyes of patients suffering from dry eye discomfort.

Treatment of DE and/or DED Via MSC-Dependent Suppression of Detrimental Immune Response in the Eyes

MSC from all tissue sources are potent immunoregulatory cells that produce a large number of immunomodulatory factors (e.g., IL-10, TGF-β, growth related oncogene (GRO), indoleamine 2,3 dioxygenase (IDO), nitric oxide (NO), interleukin 1 receptor antagonist (IL-1Ra), prostaglandin E2 (PGE2)), which can alter the phenotype and/or function of all immune cells that play a pathogenic role in the development and progression of dry eye disease, dry eye discomfort, tear hyperosmolarity, and/or tear hyperosmolarity-induced pathological changes in the eyes of patients suffering from dry eye discomfort.

By suppressing the Jak-Stat signaling pathway in T cells, MSC-sourced TGF-β can induce G1 cell cycle arrest and prevent the proliferation of these cells. MSC-derived IDO can promote expansion of immunosuppressive T regulatory cells (Tregs) and prevent their conversion in inflammatory Th17 lymphocytes.

Tregs are regulatory T cells (also referred to as “suppressor T cells”) that are generally immunosuppressive and can, for instance, help to prevent autoimmune diseases. Tregs can express several biomarkers, such as, for example, CD4 and forkhead box P3 (FOXP3). FOXP3 (also referred to as “scurfin”) is a protein that assists in regulation of regulatory pathways, including, for example, development of Tregs. Thus, the aforementioned CD4+ FOXP3+ T regulatory cells are positive for (i.e., express) both CD4 and FOXP3.

MSC-sourced NO, in an autocrine manner, can increase IDO expression in MSC and significantly enhance their immunosuppressive properties. Additionally, MSC-derived PGE2 can attenuate the proliferation of activated T cells and prevent the conversion of naïve CD4+T cells in effector Th1 and Th17 cells by suppressing IL-2 production in T lymphocytes. Moreover, MSC-sourced PGE2 can stimulate the generation of an immunoregulatory tolerogenic phenotype in DC and induce expansion of alternatively activated macrophages, contributing to the creation of an immunosuppressive microenvironment in inflamed tissues in which MSC are transplanted. Similar to PGE2, MSC-derived IL-10 and TGF-β can prevent the generation of inflammatory Th1 and Th17 cells by inhibiting the maturation of DC and by inducing the generation of alternatively activated (M2) phenotype in macrophages. Therefore, attenuated expression of co-stimulatory molecules (e.g., CD80 and CD86) and suppressed production of pro-Th1 and pro-Th17 cytokines (e.g., IL-12, IL-1β, IL-6, IL-23) can be observed in MSC-primed DC and macrophages.

In addition to T cells, DC, and macrophages, MSC are also able to efficiently inhibit proliferation and cytotoxicity of NK cells. MSC-derived TGF-β and NO can suppress the expansion of activated NK cells, while MSC-sourced IDO and PGE2 can generate the immunosuppressive and regulatory phenotype in NK cells. MSC-derived IL-10 can also down-regulate expression of pro-apoptotic and toxic molecules (e.g., perforins and granzymes) and inhibit the production of inflammatory and cytotoxic cytokines (e.g., TNF-α and IFN-γ) in NK cells, significantly reducing their cytotoxic potential.

Juxtacrine communication (e.g., direct cell-to-cell interaction between immune cells and MSC) may also be involved in MSC-dependent suppression of detrimental immunity. MSC can express pro-apoptotic molecules (e.g., programmed death-ligand (PDL)-1, PDL-2, Fas ligand (FasL)), which bind to PD and Fas receptors on the membranes of activated T and NK cells and can induce their apoptosis in a caspase-3-dependent manner.

Further, intravenously injected BM-MSC may attenuate T cell-driven ocular inflammation, thereby alleviating DED in BM-MSC-treated patients. This may result in, for instance, attenuated clinical symptoms (e.g., redness, ocular pain, dryness, scratchiness) and significantly decreased dry eye scores and/or ocular surface disease index scores. BM-MSC may prevent the activation of cytotoxic CD8+ T cells, resulting in a reduced number of CD28-expressing CD8+ T cells, which can be confirmed via, e.g., flow cytometry analysis of immune cells. Additionally, BM-MSC may alter the cytokine profile of activated CD8+T cells. DE and/or DED patients that receive MSC and/or MSC-derived products (e.g., MSC-Exos) may exhibit a significantly reduced number of pro-inflammatory, IFN-γ, and IL-2-producing CD8+ T cells, and/or an increased presence of immunosuppressive, IL-10-producing CD8+ T cells. MSC-dependent immunoregulation may therefore result in these beneficial effects in the treatment of DED. In certain instances, clinical improvements of DED-related symptoms may not occur in all BM-MSC-treated patients, which may occur if, for instance, BM-MSC are intravenously infused and are not injected directly into the eyes. In such situations, MSC-based immunomodulation would rely exclusively on the systemic effects of their secretome(s). This observation is in line with the potential therapeutic efficacy of subconjunctivally injected human BM-MSC in murine models. Subconjuctival transplantation of BM-MSC can completely attenuate detrimental immune response in MSC-treated animals. Massive intraocular infiltration of immune cells may be observed in saline-treated animals, but not in the eyes of BM-MSC-treated mice. Further, the total number of inflammatory CD3+ T cells and concentration of inflammatory TNF-α may be significantly reduced in the corneas of MSC-treated animals. Additionally, subconjunctivally injected BM-MSC can suppress the expression of, e.g., the PAX6 gene in the corneas of mice. Over-expression of the PAX6 gene can induce an altered morphology of corneal epithelial cells, increase corneal neovascularization, and promote intraocular infiltration of inflammatory immune cells. Accordingly, by potentially reducing expression of the PAX6 gene in the corneas of mice, BM-MSC can alleviate intraocular inflammation, leading to the enhanced regeneration of injured corneal epithelial cells.

Transplantation of MSC in the Eyes of DE and/or DED Patients

Due to their potent regenerative and immunoregulatory properties, MSCs from all tissue sources can be used for the treatment of many incurable degenerative, autoimmune and inflammatory diseases, including, but not limited to, dry eye disease, dry eye discomfort, tear hyperosmolarity, tear hyperosmolarity-induced pathological changes in the eyes of patients suffering from dry eye discomfort, other eye diseases, cancers, tumors, and other diseases and/or disorders. In experimental and clinical settings, MSCs can be injected either directly at the site of injury and inflammation (e.g., local transplantation) or systemically infused (e.g., intravenous, intra-arterial or intra-peritoneal injection). A majority of locally transplanted MSCs may become successfully engrafted at the site of injury where they can (i) secrete growth factors and provided trophic support to injured cells, (ii) produce immunoregulatory factors and suppress ongoing inflammation, and/or (iii) differentiate into parenchymal cells and repopulated damaged tissues. After intravenous injection, a majority of MSCs may engraft in the lungs and liver from where, in a paracrine and endocrine manner, through the activity of MSC-sourced immunomodulatory factors, the engrafted MSCs regulate detrimental immune response. Viability, phenotype, and/or function of systemically infused MSCs can be altered by cytokines to which they were exposed in systemic circulation and in the tissues of their engraftment. Therefore, significantly better therapeutic effects of MSCs may be observed after their direct transplantation in the injured and/or inflamed tissues. In line with these observations, the best therapeutic effects of MSCs in the treatment of eye diseases (e.g., dry eye disease, THO, dry eye discomfort, tear hyperosmolarity, and/or tear hyperosmolarity-induced pathological changes in the eyes of patients suffering from dry eye discomfort) can be observed where these cells were topically administered, directly in the eyes of patients.

In addition to MSCs, pluripotent stem cells (e.g., embryonic stem cells (ESC) and induced pluripotent stem cells (iPSCs)) can also be explored as a potentially valuable cell source for the repair and regeneration of injured epithelial cells in patients with various eye diseases (e.g., dry eye disease, dry eye discomfort, tear hyperosmolarity, and/or tear hyperosmolarity-induced pathological changes in the eyes of patients suffering from dry eye discomfort). Under specific culture conditions, MSCs, ESCs, and iPSCs may have a similar potential for differentiation towards corneal epithelial cells. Importantly, compared to ESCs and iPSCs, MSCs may show a superior potential for immunoregulation and suppression of detrimental immune response in the eyes of patients with THO and/or DED. However, ethical and safety issues related to the destruction of human embryos, undesired differentiation, and potential malignant transformation limit the clinical application of ESCs and iPSCs. Therefore, among all stem cells, only MSCs can be considered as novel therapeutic agents for the treatment of dry eye disease, dry eye discomfort, tear hyperosmolarity, and/or tear hyperosmolarity-induced pathological changes in the eyes of patients suffering from dry eye discomfort.

It is important to recognize and prevent any possible safety issues which could limit the clinical use of MSC. Firstly, MSC are not “immune privileged” cells since they express MHC class II molecules. Accordingly, transplantation of allogeneic MSC may aggravate strong immune response elicited during progression of DE and/or DED. Second, MSC are not exclusively immunosuppressive cells. MSC alter their phenotype and/or function under the influence of cytokines to which they are exposed. If MSC are transplanted at the ocular surface or in the vitreous body with a low level of TNF-α and IFN-γ, they can obtain a pro-inflammatory phenotype and secrete pro-inflammatory cytokines (e.g., TNF-α, IL-1β, IL-6, IL-12, IL-23), which could aggravate Th1 and Th17 cell-driven DE and/or DED. On the contrary, if MSC are engrafted in the eyes with ongoing inflammation (e.g., with the high levels of TNF-α and IFN-γ), they can acquire an immunosuppressive phenotype and produce, for instance, IDO, PGE2, IL-10, TGF-β, and/or other immunoregulatory factors that efficiently attenuate detrimental immune response. In line with these findings, there is an objective concern that MSC transplanted in the eyes with a low concentration of TNF-α and IFN-γ will obtain a pro-inflammatory phenotype and will aggravate DE and/or DED. Additionally, TGF-β and bone morphogenetic proteins (BMPs), released by macrophages and parenchymal cells in inflamed eyes, may induce unwanted chondrogenic and osteogenic differentiation of transplanted MSC. Although measurement of inflammatory cytokines and growth factors in the eyes of DE and/or DED patients prior to MSC injection will minimize the risk for MSC-dependent aggravation of DE and/or DED, it should be noted that intraocular levels of TNF-α, IFN-γ, and TGF-β can dynamically change during the progression of DE and/or DED and, therefore, concentrations of these cytokines should be continuously monitored in all MSC-treated DE and/or DED patients.

Therapeutic Potential of MSC-Exos in the Treatment of THO and/or DED

The majority of MSC-sourced immunoregulatory and growth factors that suppress detrimental immune response in the eyes and support regeneration of injured corneas, conjuctivas, Meibomian, and lacrimal glands are contained within MSC-sourced exosomes (MSC-Exos) that can be, for instance, 50-150 nm large. As cell free products, MSC-Exos address all safety concerns related to the transplantation of MSC. Furthermore, the lipid bilayer of the MSC-Exos' membrane enables ease of penetration of MSC-Exos through the corneal epithelium and across the blood-retina barrier. Accordingly, topical administration of MSC-Exos has been considered as an alternative therapeutic approach to MSC-based therapy in the treatment of dry eye disease, dry eye discomfort, tear hyperosmolarity, and/or tear hyperosmolarity-induced pathological changes in the eyes of patients suffering from dry eye discomfort.

MSC-Exos from all tissue sources are enriched with MSC-sourced microRNA (miRNAs), which bind to the RNA-induced silencing complex and inhibit gene expression in target cells. For instance, MSC-Exo-sourced miR-10a-5p and miR-10b-5p can prevent the apoptosis of injured epithelial cells, miR-191-5p can facilitate cell viability of limbal stem cells, while MSC-derived miR146a can suppress detrimental immune response by down-regulating expression of IFN-γ in Th1 lymphocytes. Labial gland MSC-Exo-sourced miR-125b can affect antibody secretion in plasma cells of patients suffering from DED secondary to Sjogren's Syndrome by modulating expression of the PR domain zinc finger protein 1 (PRDM1) gene. Accordingly, MSC-Exos may significantly reduce the percentage of activated, antibody producing, CD19+CD20−CD27+CD38+ plasma cells in peripheral blood mononuclear cells of these patients and attenuated antibody-dependent injury of lacrimal glands.

AF-MSC-Exos are enriched in neurotrophins (e.g., NGF, brain derived growth factor (BDNF)) which provide trophic support to injured neurons and promote axonal regeneration, crucially contributing to the retinal regeneration in the eyes of DE and/or DED patients.

AT-MSC-Exos also contain cytokines and growth factors that regulate lymphocyte activation (e.g., IL-10, IL-1Ra, TGF-β, GRO, soluble TNF-α receptors (sTNFRs)), and promote repair and/or regeneration of injured tissues (e.g., MMP-2 and 9). In line with these findings, MSC-Exos-mediated immunosuppression may be mainly responsible for attenuation of DED in BAC+MSC-Exo-treated mice. Specifically, AT-MSC-Exos may improve viability of injured epithelial cells by suppressing caspase-1-driven apoptosis. Additionally, AT-MSC-Exos can inhibit activation of NLR family pyrin domain containing 3 (NLRP3) inflammasome and suppress the expression of IL-1β and IL-18 in lacrimal gland-infiltrated macrophages, which can significantly reduce ocular inflammation and attenuated DED in experimental mice.

In line with these findings, topical administration of human AF-MSC-Exo-sourced eye drops in clinical settings may efficiently attenuate pain, dryness, grittiness, scratchiness, soreness, irritation, burning, watering, and/or eye fatigue in DED patients. AF-MSC-Exo-sourced eye drops can contain, for instance, IL-1Ra, sTNFRI, sTNFRII, GRO-γ, fatty acid-binding protein 1 (FABP1), and platelet factor 4 (PF4), which suppress IL-1β and TNF-α-driven inflammation, prevent the generation of inflammatory Th1 and Th17 cells, support tear stability, and reduce ocular surface epithelial damage in patients suffering from THO and/or inflammatory eye diseases (e.g., DED). AF-MSC-Exo-sourced eye drops may also promote regeneration of injured meibomian glands and restore meibomian function in patients suffering from, e.g., MGD. Before topical application of AF-MSC-Exo-sourced eye drops, meibomian ducts of MGD patients may be dilated while meibomian glands may be enlarged and tortuous with abnormal structure. The morphology of meibomian glands can accordingly significantly improve after a specific duration (e.g., 21 days) of AF-MSC-Exo-based therapy, showing the hypoilluminescent grape-like clusters. Similarly, hyperilluminescent ducts and underlying tarsus can indicate beneficial effects of AF-MSC-Exos in restoration of meibomian gland and ducts morphology. For instance, significantly improved tear film breakup time may be observed a specific duration (e.g., 21 days) after topical administration of AF-MSC-Exo-sourced eye drops, confirming restoration of meibomian gland function. Similarly, significantly improved visual acuity, relieved ocular pain, and complete healing of corneal epithelial defects may be noticed in AF-MSC-Exo-treated patients that suffer from Sjogren's syndrome. In addition to these findings, AF-MSC-Exo-sourced eye drops can improve the viability of injured corneal epithelial cells and alleviate the symptoms elicited by corneal injury. A specific course of AF-MSC-Exo-based therapy (e.g., four weeks) can remarkably improve visual acuity and significantly decrease ocular pain in patients suffering from epithelial basement membrane dystrophy with recurrent corneal erosion syndrome (RCES). Such improvement may include, for instance, no recurrence of RCES symptoms in AF-MSC-Exo-treated patients after four months, suggesting beneficial effects of AF-MSC-Exos in the repair and regeneration of injured corneal epithelial cells. Importantly, AF-MSC-Exo-sourced eye drops ae believed to be generally well tolerated in clinical settings, without any side effects.

Therapeutic Potential of MSC-Exos May Depend on Tissue Origin of their Parental Cells

It should be noted that the content, and therefore, therapeutic potential of MSC-Exos may depend on the tissue origin of their parental cells. For instance, BM-MSC-Exos may be enriched with immunoregulatory factors that induce the generation of an immunosuppressive phenotype in macrophages (e.g., TGF-β, IL-10), protect from oxidative stress-induced injury (e.g., miR-214), attenuate TNF-α and IL-1β-driven inflammation (e.g., sTNFRI, sTNFRII, IL-1Ra), promote expansion of Tregs, and prevent IL-23-dependent generation of Th17 cells (e.g., IDO, Kynurenine, TGF-β). UC-MSC-Exos may contain the enzymes manganese superoxide dismutase and glutathione peroxidase 1, which have anti-apoptotic and anti-oxidation abilities and are capable of preventing oxidative stress-induced injury of neural cells in the eyes of DE and/or DED patients. Additionally, UC-MSC-Exos may reduce nerve inflammation since they are enriched in proteins which block the degradation and proliferation of the NFκB inhibitor IκBα. UC-MSC-Exos can also contain mir-21, miR-23a, miR-125b, and/or miR-145, which inhibit fibrosis by affecting the factor-β2/SMAD2 pathway, and mir-135b-5p and/or mir-499a-3p, which regulate angiogenesis. AF-MSC-Exos may contain proteins that modulate neurodevelopment and lymphocyte activation (e.g., A disintegrin and a metalloprotease (ADAM)-9, ADAM-10), repair and regeneration of injured tissues (e.g., MMP-2 and 9), and are enriched in proteins that regulate oxidative stress e.g., (peroxiredoxin-1,-2,-4,-6). AF-MSC-Exos can also contain neurotrophins, which provide trophic support to injured neurons in the eyes of DE and/or DED patients. Therefore, AT-MSC-Exos may have similar therapeutic potential for DE and/or DED treatment as other tissue source MSC-Exos. AT-MSC-Exos may also be enriched with immunoregulatory proteins, such as, for example, IDO, TGF-β, IL-10, IL-1Ra, and PGE2, which suppress Th1 and Th17 cell-driven inflammation and NK cells-dependent injury of epithelial cells in DE and/or DED patients. The main advantage of AT-MSC-Exos is their high availability, since they are easily derived from patients' AT. Accordingly, AT-MSCs may be an alternative when MSC-Exos from other sources are difficult to extract or are not suitable for therapy.

Although MSC-Exos from all tissue sources represent potentially effective therapeutic agents in regenerative ophthalmology, the exact therapeutic dose of MSC-Exos for treatment of various eye diseases is still unknown. Therefore, the optimal dose, frequency, and treatment schedule must be determined before MSC-Exos can be offered as a new remedy for the treatment of such eye diseases. Additionally, the exact growth and immunoregulatory factor(s) which is/are mainly responsible for the beneficial effects of MSC-Exos should be defined. Afterwards, MSCs could be genetically engineered to over-express these factors, which will be contained at high concentrations in MSC-Exos. Administration of MSC-Exos enriched with the most effective bioactive factor(s) will enhance the therapeutic potential and efficacy of MSC-Exos in the treatment of various eye diseases.

MSC-Exos, and/or any compound and/or formulation containing MSC-Exos (e.g., d-MAPPS pharmaceutical compositions including, for instance, d-MAPPS solutions), which contain all MSC-sourced growth factors and immunoregulatory proteins, can easily bypass all biological barriers in the eyes due to their nano-size dimension and lipid envelope. Accordingly, such MSC-Exos can deliver their cargo directly in corneal epithelial cells and eye-infiltrated leukocytes. As a cell-free agent, MSC-Exos address all relevant safety issues related to the transplantation of their parental cells, including the risk of unwanted differentiation and aggravation of intraocular inflammation.

Accordingly, in at least one embodiment of the disclosure, d-MAPPS pharmaceutical compositions (e.g., d-MAPPS solutions) containing one or more MSCs and/or one or more MSC-Exos are disclosed. Such d-MAPPS pharmaceutical compositions may include any one or more types of MSCs described herein, one or more types of MSC-Exos described herein, and/or one or more MSC-sourced growth factors and/or immunoregulatory proteins described herein.

### Impact of Ion Channels on DED Development and Progression

Ion channels (transient receptor potential ankyrin 1 (TRPA1), transient receptor potential melastatin 8 (TRPM8) and transient receptor potential vanilloid 1 (TRPV1)) are crucially important for maintaining cellular ion homeostasis and are intimately involved in the regulation of ocular surface health. Therefore, changes in their structure and/or function result in changes in tear osmolarity, representing an initial step in the pathogenesis of DED.

In other embodiments, TRPA1 is an ion channel that controls influx of calcium cations through the cell membrane. It is highly expressed in the trigeminal nerve which controls sensation in the face and eyes. In embodiments, results obtained in animal model of DED showed that activation of TRPA1 resulted in decreased tear production, more frequent blinking, and forelimb eye wipe behavior.

In other embodiments, TRPM8, a nonselective cation channel, plays a crucial role in DED. It's activated by tear hyperosmolarity, due to tear film evaporation. Upon activation, TRPM8 causes a temporary rise in intracellular calcium levels. TRPM8 is vital for stimulating the lacrimal glands to secrete tears. Lower levels of TRPM8 in the cornea are linked to reduced corneal sensitivity and tear production. Conversely, increasing TRPM8 levels boosts tear secretion without triggering pain. The age-related decline in TRPM8+ neurons could explain the prevalence of DED in older individuals. Inhibition of TRPM8 reduces basal tear production, highlighting its critical role in tear generation. TRPM8 further contributes to DED by negatively regulating the activity of TRPV1, which is a nonselective cation channel found on sensory neurons that plays a crucial role in pain perception and neurogenic inflammation. When activated, TRPV1 allows large cations to enter the cell, triggering a cascade of events including calcium influx, channel relocation and cytokine production. TRPV1 is activated by stimuli like capsaicin, protons, heat, and inflammatory factors. In the context of DED, TRPV1 activation in corneal epithelial cells leads to the release of pro-inflammatory factors such as IL-6 and IL-8 which acts synergistically with tumor necrosis factor alpha (TNF-α) and IL-1β to enhance expression of E and P selectins on endothelial cells (ECs) enabling massive influx of circulating leukocytes in inflamed eyes.

Chronic DED animal models show increased TRPV1 expression in both corneal nerve fibers and the trigeminal ganglia, suggesting a link between TRPV1 and DED development. Moreover, increased TRPV1 expression in TRPM8+ neurons might contribute to cold allodynia, a common symptom in DED. Animal models of DED demonstrate that activating TRPV1 leads to various symptoms, including increased eye wiping behavior, noncohesive responses, spontaneous blinking, orbicularis oculi muscle activity, and inflammation. Blocking TRPV1 in these models reduces sensitivity to acidic pH. This sensitivity to acidic pH is further mediated by acid-sensing ion channels (ASICs). In DED animal models, ASIC1 and ASIC3 mRNA levels are elevated. Activating ASIC3 in the eye causes corneal sensory neurons to fire and increases pain responses to acidic stimuli. Blocking ASIC3 reduces these responses. However, even with ASIC3 knockdown, a compensatory mechanism involving ASIC1a still triggers responses to acidic stimuli in DED. Clinical trials using siRNA to block TRPV1 in DED patients have shown promising results. These trials demonstrated a reduction in pain (measured by Visual Analogue Scale (VAS)), conjunctival hyperemia, and Ocular Surface Disease Index (OSDI) scores, highlighting the role of TRPV1 in DED and its potential as a therapeutic target.

Importantly, stimulation of TRPM8 inhibits capsaicin-induced TRPV1 activation in various eye tissues. This suggests that TRPM8 controls generation of inflammatory response in DED patients. Animal models demonstrate that TRPM8 agonists reduce eye-wiping behavior and muscle activity, while antagonists increase TRPV1 expression and muscle activity. Clinical trials on DED patients have shown that TRPM8 agonists and/or TRPV1 antagonists are promising therapeutic options, offering significant improvements in DED-related symptoms.

Furthermore, TRPM8 and TRPV1 channels interact with sodium and potassium channels, influencing their expression. Blocking TRPM8 in DED animal models reduced the mRNA levels of sodium channels Nav1.7 and NaV1.8. Similarly, blocking TRPV1 in another animal study led to reduced mRNA levels of these sodium channels. A recent study using optogenetic inhibition of Nav1.8 expressing corneal afferents in DED animal models also resulted in decreased pain. In other embodiments, animal studies have also revealed a connection between TRPV1 and the potassium channel KCJN6 in DED. Blocking TRPV1 reduced the expression of KCJN6 in DED mice. Interestingly, blocking TRPM8 also decreased the expression of this potassium channel.

In embodiments, the present data suggests a link between DED and MGD and the altered function of Sodium-potassium-chloride cotransporter (NKCC) and Sodium potassium ATPase (NKA). NKCC is a protein involved in secondary active transport, facilitating the movement of sodium, potassium, and chloride into cells. Studies on corneal epithelial cells show that hyperosmotic stress and epidermal growth factors induce NKCC expression and activity, highlighting its role in osmoadaptation. NKA, another important protein, is involved in active transport across the cell membrane. It pumps sodium out of the cell and potassium into the cell. Reduced NKA expression has been observed in aged meibomian glands, and NKA agonists have been shown to increase NKA expression and promote meibocyte proliferation in mice.

In other embodiments, TRPV1 also interacts with purinergic receptors (P2X and P2Y) involved in cellular signaling. These receptors are activated by extracellular ATP, a molecule released during tissue injury and inflammation. P2X receptors, ligand-gated ion channels permeable to sodium, potassium, and calcium, are found in increased numbers on immune cells at sites of inflammation. ATP activation of P2X receptors on these cells triggers the release of inflammatory cytokines, exacerbating the inflammatory response. In DED, P2X7R blockade in animal models reduces orbicularis oculi muscle activity, a marker of DED-associated discomfort. This suggests that P2X7R contributes to the pain and muscle spasms experienced by DED patients. P2Y2 receptors, when activated by ATP, stimulate chloride ion and fluid secretion, as well as mucin release from conjunctival goblet cells. Interestingly, P2Y2 activation can also indirectly activate TRPV1, contributing to inflammatory pain. However, in DED animal models, P2Y2 agonists have been shown to increase tear protein secretion and glycoprotein release by goblet cells, suggesting a potential therapeutic role.

Similarly, results obtained in DED animal models showed that GABAA receptor agonists reduced orbicularis oculi muscle activity induced by hypertonic saline, while antagonists enhanced this activity. The epithelial sodium channel (ENaC) plays a crucial role in maintaining salt and water balance. The NMDA receptor, an important excitatory neurotransmitter receptor, is also involved in corneal reflexes. In DED animal models, blockade of NMDA receptors reduces hypertonic saline-induced orbicularis oculi muscle activity, suggesting its role in pain and discomfort.

Aquaporins (AQPs), transmembrane channels facilitating water and small solute transport across cell membranes, play a crucial role in regulating tear production. AQP5 is essential for lacrimal gland fluid secretion. Hyperosmotic stress, a hallmark of DED, triggers increased AQP5 mRNA expression in human corneal epithelial cells, promoting transepithelial fluid efflux and protecting corneal barrier function during tear hyperosmolarity. An increased AQP4 and AQP5 mRNA expressions were observed in the early stages of DED, suggesting a potential compensatory mechanism.

Concurrently, cystic fibrosis transmembrane conductance regulator (CFTR), a cAMP-activated chloride anion channel, is expressed in corneal and conjunctival epithelial cells. Studies have revealed an increased accumulation of chloride ions in conjunctival impression cytology samples from DED patients, indicating potential dysfunction of chloride ion channels like CFTR. CFTR activation significantly improved tear secretion in both healthy and inflamed eyes of DED animal models. Furthermore, CFTR activation has been demonstrated to ameliorate epithelial disruption and increase ocular surface fluid secretion in DED animal models, highlighting its potential as a therapeutic target for DED. These findings suggest alterations in the structure and/or function of ion channel receptors and consequent hyperosmolarity of the tears represent the initial step in the development and progression of DED.

Signaling Pathways which are Responsible for Hyperosmolarity-Dependent Generation of Detrimental Inflammatory Response in the Eyes of DED Patients

In embodiments, it is established that TRPA1 and TRPV1 ion channels play significant roles in the modulation of tissue inflammatory and immune responses and that these roles often rely upon extensive interactions between sensory neurons and immune cells. A higher propensity of TRPV1-expressing corneal sensory nerves was observed after corneal injury, along with their interaction with intra-epithelial leukocytes and professional antigen-presenting dendritic cells (DCs).

In embodiments, corneal parainflammation, a low-grade inflammatory response to tissue stress that lies between the basal homeostatic state and symptomatic inflammation, have been demonstrated to occur during lens wear in humans, leading to alterations in the number of intraepithelial DCs. It has been established that both TRPA1 and TRPV1 ion channels are essential not only for contact lens-induced corneal parainflammatory responses but also for maintaining baseline levels of certain resident corneal immune cells. Remarkably, the absence of lens wear results in a significant decrease in the number of resident MHC class II+ cells in corneas of TRPA1 knockout mice (by approximately 50% compared to wild type (WT), and further reduced by approximately 90% compared to WT in TRPV1 knockout corneas), indicating a unique influence of each ion channel on these cells. Furthermore, TRPA1 and TRPV1 ion channels are implicated in lens-induced parainflammation mediated by IL-17-producing Th17 cells, which are necessary for optimal anti-microbial, neutrophil-dependent immune responses in the corneas of individuals wearing lenses.

In other embodiments, hyperosmolarity triggers the activation of the TRPV1-dependent signaling pathway in corneal epithelial cells, leading to the activation of transcriptional factor nuclear factor kappa B (NF-κB). This, in turn, causes an increase in the expression of genes for TNF-α and IL-1β, resulting in enhanced production of these inflammatory cytokines. IL-1β and TNF-α stimulate various kinases (mixed lineage kinase (MLK)-2/-3, transforming growth factor β-activated kinase 1 (TAK1), mitogen-activated protein kinase (MKK)-1, apoptosis signal-regulating kinase 1 (ASK1), MKK7, and Jun N-terminal kinase (JNK)) in eye-infiltrated, professional antigen-presenting DCs. These kinases phosphorylate and activate transcription factors c-Jun and ATF2. Activated c-Jun and ATF2 move to the nucleus, where they increase the expression of pro-Th1 (IL-12) and pro-Th17 cytokines (IL-1β, IL-6, IL-23) in DCs. Through the production of IL-12, DCs stimulate signal transducers and activators of (STAT)-1 and T-bet transcriptional factors in naïve CD4+ T lymphocytes, leading to their differentiation into effector, interferon gamma (IFN-γ)-producing Th1 cells. In a similar manner, by stimulating STAT-3 and RAR-related orphan receptor gamma T (RORγT) transcriptional factors in naïve CD4+T cells, DC-derived IL-1β, IL-6, and IL-23 promote the development and expansion of inflammatory CD4+Th17 cells.

In the eyes of DED patients, Th1 cell-derived interferon gamma (IFN-γ) and Th17 cell-sourced IL-17 and IL-22 induce generation of inflammatory M1 phenotype in macrophages and N1 phenotype in neutrophils, crucially contributing to the generation innate cell-driven inflammation. N1 neutrophils and M1 macrophages are valuable sources of inflammatory mediators, which contribute to the reduced expression of mucins, apoptotic death of corneal and conjunctival epithelial cells, and to the loss of goblet cells in inflamed eyes of DED patients. Additionally, M1 macrophages and N1 neutrophils produce Th1 and Th17 cell-attracting chemokines (C—X—C motif ligand (CXCL)-9/-10, C—C motif ligand (CCL)-20), enabling massive influxes of CD4+ and CD8+ Th1 and Th17 cells in inflamed eyes of DED patients.

In embodiments, Th1 and Th17 lymphocytes, by generating inflammatory cytokines (IFN-γ and IL-17), cause damage to the corneal epithelial barrier, trigger inflammation of the meibomian glands, and reduce the production of tears in DED mice. Additionally, blocking antibody-dependent neutralization or genetic removal of IFN-γ and IL-17 significantly reduces eye inflammation and almost completely stops damage to the corneal epithelial barrier in DED mice, proving the essential role of these inflammatory cytokines in DED development. IL-17 produced by Th17 cells increases the expression of MMP-3 and MMP-9, which in turn leads to the breakdown of the corneal epithelial barrier.

Additionally, Th1 cell-derived IFN-γ activates both extrinsic and intrinsic apoptotic pathways in corneal epithelial cells. The extrinsic or “death receptor” pathway involves the interaction between Fas ligand (FasL)- and TNF-related apoptosis-inducing ligand (TRAIL)-activated caspase-8, while the intrinsic or “mitochondrial” pathway is activated by caspase-9. While the administration of exogenous IFN-γ significantly increased caspase-3, -8, and -9-dependent apoptosis in conjunctival epithelial cells, the pharmacological inhibition or genetic deletion of IFN-γ significantly reduced immune cell-induced damage to conjunctival epithelial and goblet cells in DED mice. Similar outcomes have been observed in DED patients; a disturbance in the corneal barrier associated with IL-17 was found to increase the likelihood of corneal ulcers and vision loss, whereas higher levels of IFN-γ were linked to the development of conjunctival epithelial squamous metaplasia, resulting in a progressive loss of goblet cells.

### Tear Hyperosmolarity Because of Inflammation-Induced Modification of Ion Channels: Creation of a Vicious Circle in the Eyes of DED Patients

Severe DED, which develops due to the enhanced Th1 and Th17 cell-driven eye inflammation, results in the sensation of pain due to primary stimulation and sensitization of polymodal nociceptors in the inflamed eyes. Conversely, in moderate DED, cold thermoreceptors are being primarily activated, leading to a predominant sensation of unpleasant dryness. Mediators released due to local inflammation increase the excitability of eye polymodal nociceptors, thereby intensifying pain sensations. Over time, chronic inflammation can lead to further, long-lasting alterations in the expression and function of stimulus-transducing and voltage-sensitive ion channels, thereby modifying the excitability of polymodal terminal receptors and evoking chronic inflammatory pain.

In embodiments, injured cells within the inflamed eye and activated immune cells secrete various bioactive factors, including hydrogen ions (H+), adenosine triphosphate (ATP), adenosine, protons, prostaglandins, leukotrienes, bradykinin, serotonin, platelet-activating factor, histamine, and inflammatory cytokines which stimulate spontaneous impulse activity in nociceptor terminals, reducing the threshold for stimulus detection and increasing the amplitude of nerve impulse responses elicited by suprathreshold stimulation.

In other embodiments, the sensitization of peripheral nociceptors is responsible for the unique quality and persistence of pain experienced from inflamed tissues. Inflammatory factors, including prostaglandins (PGE2), bradykinin, serotonin, TNF-α, IL-1β, IL-6 and IL-8, bind to their respective receptors (neurotrophic tyrosine kinase receptors (NTKR), including TrkA, and/or G-protein coupled receptors (GPCRs), such as bradykinin and PGE2 receptors, and cytokine receptors), activating multiple intracellular pathways. These pathways include Protein Kinase A (PKA), Protein Kinase B (PKB), Protein Kinase C (PKC), Mitogen-activated protein Kinase (MAPK), and the Ca++/Calmodulin-dependent Kinase I and II (CamKI/II). Activation of these signaling pathways results in the post-translational modification of ion channel proteins, affecting their function.

Post-translational modifications exert a broad range of effects on ion channel proteins, varying from highly stable modifications to those that are transient and reversible. For example, glycosylation and the formation of disulfide bonds are directly involved in the synthesis, maturation, and folding of proteins. The covalent binding of molecules, such as the addition of ubiquitin moiety, facilitates more rapid and stable modifications. Furthermore, the incorporation of a phosphoryl group, which carries two negative charges at physiological pH, can alter the protein's structure and function by modifying the free energy landscape.

Post-translational modifications of voltage-gated sodium channels (Navs) are crucial for the development of tear-hyperosmolarity-dependent DED. Navs are activated in response to depolarization of the transmembrane voltage, leading to the generation of a fast, transient, and significant inward sodium current, which is responsible for the ascending phase of the action potential. Navs contain charged residues in the voltage sensor domain that are sensitive to changes in membrane potential. Upon a change in transmembrane voltage, these charged domains shift in the electric field, resulting in conformational changes known as gating. The addition of charged groups to their intracellular, extracellular, or transmembrane domains modifies the intrinsic properties and functions of the protein. In addition to the direct electrostatic influence on gating, phosphorylation can also create or disrupt binding sites for interaction with other regulatory proteins that affect Nav function. The molecular mechanisms underlying inflammation-driven post-translational modifications of Navs involve the activation of various intracellular kinases. Binding of PGE2 and bradykinin to GPCRs predominantly leads to the activation of PKA and PKC kinases through the adenylate cyclase (AC) and inositol 3-phosphate (IP3) secondary messengers, respectively. Binding of NTKR and CR to TNFα, IL-6, and NGF activates ERK1/2 and p38 kinases via different potential secondary messengers. These signal transduction pathways can also undergo crosstalk with one another. The increased activation of kinases, the downregulation of Nedd4-2, and the accumulation of methylglyoxal result in enhanced function or expression of Nav1.7/Nav1.8 channels at the cell membrane, leading to increased sodium influx and, consequently, to nociceptive neuronal hyperexcitability. In a similar manner, potassium channels may also be activated because of inflammation-induced post-translational modifications of protein subunits.

In other embodiments, by inducing structural and functional changes in ion channels, long-lasting and un-treated, detrimental immune cell-driven inflammatory response causes hyperosmolarity of the tears and generates a vicious circle in the eyes that aggravates all DED-associated signs and symptoms. These findings suggest that suppression of Th1 and Th17 cell-driven eye inflammation and alleviation of tear hyperosmolarity through the modulation of ion channels in the eyes of DED patients hold promise for developing new therapeutic strategies for managing DED.

Therapeutic Potential of d-MAPPS® in the Attenuation of Inflammation and Hyperosmolarity-Induced Pathological Changes in the Eyes of DED Patients

In embodiments, d-MAPPS® is a hypo-tonic ophthalmic solution which contains large number of immunoregulatory, angio-modulatory and trophic factors and is to efficiently attenuate on-going eye inflammation and tear hyperosmolarity, promoting enhanced tissue repair and regeneration. Precisely, d-MAPPS® contains interleukin 1 receptor antagonist (IL-1Ra), soluble receptors of tumor necrosis factor alpha (sTNFRI, sTNFRII), Growth-Related Oncogene gamma (GRO-γ), fatty acid-binding protein 1 (FABP1) and Platelet factor 4 (PF4) which alleviate eye inflammation, support tear stability and prevent ocular surface epithelial damage, contributing to the enhanced repair and regeneration of ocular surface epithelial barrier in DED patients.

In other embodiments, as a hypo-tonic ophthalmic solution, d-MAPPS® has been shown to effectively stabilize the tear film, thereby mitigating the symptoms of DED. It works by restoring the natural osmolarity of the tear film, reducing cellular stress, and indirectly supporting the body's ability to regulate inflammation without the introduction of exogenous proteins or cytokines. d-MAPPS® functions by restoring the natural osmolarity of the tear film, a key factor in alleviating the cellular stress associated with DED. By stabilizing the tear film, this solution not only reduces the direct symptoms of irritation and dryness but also impacts the broader inflammatory pathways, reducing the need for exogenous anti-inflammatory agents. It seems that among various bioactive factors, d-MAPPS® containing FABP1 protein was mainly responsible for these beneficial effects of d-MAPPS® in the attenuation of tear hyperosmolarity. FABP proteins regulate trans-epithelial water transport and maintain epithelial barrier at ocular surface. Accordingly, down-regulated levels of FABP proteins were noticed in the tears of patients suffering from DED and reduced expression and production of FABP proteins are associated with increased tear evaporation. Since d-MAPPS® contains high concentration of FABP1 protein, d-MAPPS® may, in FABP1-dependent manner, regulate trans-epithelial water transport, support tear stability and prevent ocular surface epithelial damage in the eyes of DED patients, resulting in the alleviation of dryness, grittiness, scratchiness and soreness. By preventing hyperosmolarity-induced changes in ion channels, d-MAPPS® may suppress hyperosmolarity-dependent activation of NF-κB n corneal epithelial cells and eye-infiltrated DCs, resulting in the reduced expression of genes which regulate synthesis of inflammatory cytokines TNF-α, IL-1β and IL-12.

In addition, d-MAPPS® contains IL-1Ra. sTNFRI and sTNFRII directly suppress IL-1β and TNF-α-driven recruitment of circulating leukocytes and TNF-α and IL-1β-dependent alterations of ion channels, reducing hyperosmolarity and total number of immune cell at ocular surface. When d-MAPPS® containing IL-1Ra bind to the IL-1 receptor (IL-1R) and on endothelial cells of the eyes of DED patients, binding of IL-1β to IL-1R is blocked and pro-inflammatory signals from IL-1R are stopped. Accordingly, various pro-inflammatory events, initiated by IL-1β:IL-1R binding, including the synthesis and releases of chemokines, enhanced influx of leucocytes and post-translational modifications of ion channels in inflamed eyes, are inhibited d-MAPPS containing IL-1Ra. Similarly, d-MAPPS® containing sTNFRI and sTNFRII bind to TNF-α and prevent TNF-α-dependent pathological events in the eyes of DED patients.

In embodiments, d-MAPPS® effectively reduces the concentration of IL-12 in the supernatants of human peripheral blood mononuclear cells (pbMNCs) and reduces the production of IFN-γ in activated immune cells. d-MAPPS® containing GRO-γ inhibits DCs: T-cell interactions and successfully attenuates the expansion of inflammatory Th1 immune cells that rely on DC-sourced IL-12. GRO-γ-treated DCs show a ‘tolerogenic’ feature with higher IL-10 and lower IL-12 and IFN-γ production. Therefore, it is highly expected that intraocular administration of d-MAPPS® affects the crosstalk between antigen-presenting, IL-12-producing DCs and naive CD4+ T cells in lymph nodes, preventing DC-dependent generation of IFN-γ-producing Th1 lymphocytes. Since IFN-γ-producing T-cells increase the inflammatory capabilities of macrophages and push them towards inflammatory M1 cells, by delivering GRO-γ, d-MAPPS® inhibits the activation of intraocular M1 macrophages and reduces M1 macrophage-driven inflammation in the eyes of DED patients. Moreover, by suppressing TNF-α, IL-1β and IL-12-dependent activation of eye-infiltrated DCs, generation of inflammatory Th1 lymphocytes and M1 macrophages, d-MAPPS® attenuate tear hyperosmolarity caused by post-translational modifications, structural and functional changes of voltage-gated sodium channels that could develop due to the detrimental effects of DCs/Th1 cell/M1 macrophage-sourced inflammatory mediators (PGE2, bradykinin, serotonin, TNF-α, IL-1β, IL-6 and IL-8).

In embodiments, by addressing both tear hyperosmolarity and detrimental inflammatory response through natural cytokine modulation, hypotonic and immunomodulatory d-MAPPS® ophthalmic solution should be considered as new therapeutic agent which represents novel therapeutic approach for DED treatment. In line with these observations are findings obtained in pilot clinical trials which showed that d-MAPPS® successfully reduced eye discomfort and pain in 131 patients with DED, consisting of 27 men and 104 women, with an average age of 62 years (ranging from 19 to 85). Patients who received d-MAPPS® experienced a substantial improvement in their symptoms, as evidenced by the significant drop in VAS and SPEED scores 3 months after starting treatment, with the maximum improvement seen after a year of treatment, showing its long-term positive impact on reducing eye symptoms in patients with DED (FIG. 1). Crucially, d-MAPPS® was well accepted by all patients. No adverse reactions were reported by the 131 patients treated with d-MAPPS®, indicating that the topical administration of d-MAPPS® ophthalmic solution should be considered as a new, safe and efficacious method for treating DED.

In other embodiments, d-MAPPS® promoted regeneration of injured meibomian glands and efficiently attenuated DED-related symptoms in a patient suffering from MGD. Prior to the administration of d-MAPPS®, the ducts of MGD patient were widened and the glands themselves were larger and twisted in an atypical way. The shape of the glands was notably enhanced after three weeks of therapy with d-MAPPS®, with the appearance of dim, grape-like clusters at the base of the glands. This change was also observed in the ducts, with a return to a more normal shape. Prior to the d-MAPPS® treatment, a patient with MGD felt a sensation of a foreign body in the eye, along with discomfort, irritation, dryness, a burning feeling, and fatigue. However, after three weeks of using d-MAPPS®, there were no reports of these, DED-related issues by the patient. Three weeks after starting the therapy, there was an improvement in the time it took for the tears to break up, signifying the improvement of the function of the meibomian glands. There were no reported issues such as eye pain, continued bleeding, or infections during or after the d-MAPPS®-based treatment. The patient reported no negative effects from the d-MAPPS®-based treatment, indicating that d-MAPPS® is safe for use in the eye. Incorporating the insights from this understanding transforms DED management by emphasizing the importance of osmotic and cytokine balance. This approach not only addresses the immediate symptoms but also the inflammatory processes at the core of DED, signalling a shift towards therapies that offer systemic modulation of the tear film's biochemistry.

### Further Factors Contributing to THO and DED

Further to the above, various factors contribute to the development of THO and DED. Hormones, medicaments, environmental factors, dysfunctional neural regulation of lacrimal gland secretion, poor aqueous tear flow, and excessive tear evaporation result in the development of THO. THO induces increased activation and maturation of dendritic cells, which generates a detrimental Th1 and Th17 cell-driven immune response in the eyes of DED patients. Through the production of inflammatory cytokines, Th1 and Th17 cells induce inflammation, which causes injury to the epithelial and goblet cells in the eyes of DED patients. The loss of goblet, corneal, and conjunctival epithelial cells results in excessive tear evaporation and tear film instability, which crucially contribute to the development of THO and DED.

Dryness, grittiness, scratchiness, discomfort, inflammation, burning, watering, a feeling of a foreign substance in the eye, ocular fatigue, pain, and decreased functional visual acuity are all common symptoms of DED. Eye dysfunction drastically attenuates patients' quality of life and impairs patients' abilities to do daily tasks that depend on their vision. Long-term symptom relief is currently the main therapeutic goal of DED treatment, along with reestablishing the natural equilibrium on the surface of the eye. Accordingly, tear replacement medications are increasingly offered to DED patients in order to attenuate dry eye-related symptoms. However, the complex etiology of DED necessitates a more comprehensive therapeutic approach that can interfere with the intracellular signaling pathways, which are responsible for the development of pathological changes in the eyes of DED patients. Therefore, a better understanding of the pathophysiological processes that regulate the development and progression of DED is crucially important for the establishment of new and more effective DED-related treatment approaches, which would significantly improve the quality of life of DED patients.

A large number of experimental and clinical studies have demonstrated that tear hyperosmolarity (THO) plays a crucially important role in the development and progression of DED. Accordingly, osmoprotectants are frequently used to counteract the ocular surface damage caused by a hyperosmolar tear film. Osmoprotectants act as “compatible solutes” that enter the cells, stabilize the cell membranes, maintain cellular hydration, and, due to their anti-oxidant and anti-inflammatory properties, protect the eye from oxidative stress and inflammation-induced injury. L-carnitine, a substance found in osmoprotectants, has been shown to inhibit the activity of the transient receptor potential vanilloid 1 (TRPV1) ion channel in human corneal epithelial cells (CECs), suppress hypertonicity-induced increases in intracellular calcium ions (Ca²⁺), and prevent cell shrinkage. Since increased and prolonged activity of the TRPV1 channel is associated with the aggravation of DED-related symptoms, topical administration of L-carnitine-containing osmoprotectants could have beneficial effects in DED treatment.

Although tear osmolarity testing is frequently used as a tool for DED diagnosis and treatment monitoring, the results of one recently conducted multi-center clinical study (“Dry Eye Assessment and Management (DREAM)”) showed only a weak correlation between tear osmolarity and DED-related symptoms. In order to delineate the role of tear osmolarity in the pathogenesis of DED, in this review article, we summarized current knowledge related to the molecular mechanisms responsible for the development of tear hyperosmolarity (THO)-induced pathological changes in the eyes of DED patients and we briefly discussed the therapeutic potential of hypo-osmotic eye drops in DED treatment. In June 2023, a comprehensive literature review was undertaken using many databases (MEDLINE, EMBASE, and Google Scholar). The keywords used in our selection were: “dry eye disease”, “keratoconjunctivitis sicca”, “dysfunctional tear syndrome”, “tear hyperosmolarity”, “eye inflammation”, “immunomodulation”, and “therapy”. All journals were considered and the initial search retrieved 112 articles. The abstracts of all these articles were subsequently reviewed by two of the authors (CRH and VV) independently to check their relevance to the subject of this manuscript. Eligible studies had to delineate the molecular and cellular mechanisms responsible for the THO-related pathological changes in the eyes of DED patients, and their findings were analyzed in this review.

### Additional Mechanisms of Action Responsible for THO Development

Tears are a lacrimal gland-sourced salty solution that lubricates and protects the corneal epithelium and inner surface of the eyelids from foreign particles and infectious agents. THO is caused by alterations in tear composition and production in cases of poor aqueous tear flow, excessive tear evaporation, or a combination of these occurrences. The composition of tears may vary based on their purpose. Basal tears contain water, electrolytes, and anti-bacterial proteins (lysozyme, lactoferrin, and lipocalin), which destruct bacterial cells walls or bind to iron, making it unavailable to the invading pathogens. Emotional tears, on the other hand, contain higher levels of stress hormones and other chemicals, including leucine and enkephalin, which may reduce pain and improve mood. Additionally, emotional tears contain prolactin, which promotes the synthesis of immunoglobulin A (IgA) in plasma cells and enhances the anti-bacterial humoral immune response in the eyes. Alterations in the concentration of electrolytes, proteins, and other solutes in the tears leads to the changes in tear osmolarity and represents one of the first indicators of lacrimal gland dysfunction.

### Further Impacts of Dysfunctional Neural Regulation of Lacrimal Gland Secretion on the Development of THO and DED Progression

Dysfunction in the neural control of glandular tear secretion plays a significant role in the development of THO and DED progression. When sensory neurons in the cornea and conjunctiva detect changes in osmolarity, they transmit signals to the lacrimal glands, initiating tear secretion. A large number of experimental and clinical studies have demonstrated that transient receptor potential (TRP) channels are mainly responsible for the neural regulation of tear production. TRP channels belong to a heterogeneous superfamily of widely expressed, functionally varied cation channels that display intricate regulation patterns and are sensitive to a variety of stimuli, including TH. Among various TRP channels, the transient receptor potential melastatin 8 (TRPM8) and transient receptor potential vanilloid 1 (TRPV1) are dominantly expressed in sensory neurons of the cornea and conjunctiva. TRP channels are essential for normal neuronal functioning and play a crucial role in sensory perception, neuronal excitability, and synaptic transmission. The activation of TRP channels in presynaptic terminals can modulate the release of neurotransmitters, affecting synaptic transmission and neuronal communication.

It should be emphasized that TRP channels are also expressed in non-neuronal corneal cells and in all three layers of the cornea. These channels are essential for maintaining the health and functionality of the cornea, contributing to clear vision and overall ocular well-being. In addition to sensory transduction, TRP channels are involved in maintaining corneal integrity and promoting the repair and regeneration of injured tissue. TRP channels affect the proliferation, differentiation, and migration of corneal epithelial and stromal cells, enabling remodeling of the extracellular matrix and wound healing. Furthermore, TRP channels, particularly TRPM8 and TRPV4, are involved in regulating tear secretion and osmolarity. Activation of these TRP channels can stimulate tear production and maintain a stable tear film, preventing dry eye conditions.

In addition to sensory transduction, TRP channels play a crucial role in the regulation of corneal integrity, wound healing, and tear film homeostasis. Although there are distinctly different functions of TRP channels between neuronal and non-neuronal cells, the activation mechanisms are the same. After TRP channel activation, calcium release occurs through various pathways, depending on the specific TRP channel and cellular context. Some TRP channels are involved in the regulation of store-operated calcium entry. In this process, TRP channels act as calcium-permeable channels located on the plasma membrane. When the endoplasmic reticulum (ER) calcium stores are depleted, usually due to receptor-mediated activation of phospholipase C (PLC) and subsequent inositol trisphosphate (IP3) production, the stromal interaction molecule (STIM) undergoes conformational changes and migrates to regions of the ER close to the plasma membrane, where it activates TRP channels. This activation allows Ca²⁺ ions to enter the cytoplasm through the TRP channels, replenishing the depleted ER stores. Some other TRP channels can directly allow Ca²⁺ ions to enter the cell upon activation. These channels have a high permeability to calcium and are often referred to as calcium-permeable TRP channels. Activation of these TRP channels leads to conformational changes that open the channel pore, allowing Ca²⁺ ions to flow into the cytoplasm from the extracellular environment. The influx of calcium through these TRP channels can rapidly increase cytoplasmic calcium levels, triggering various cellular responses.

In embodiments, following the evaporation of the tear film, activated TRPM8 receptors allow Ca²⁺ ions to enter the cell. The increased intracellular calcium concentration activates calcium-dependent protein kinases (protein kinase C (PKC) and calcium/calmodulin-dependent protein kinase II (CaMKII)). PKC and CaMKII phosphorylate downstream target proteins, resulting in the activation of transcriptional factors (nuclear factors of activated T cells (NFAT) and cAMP response element-binding proteins (CREB)), which enhances the expression of genes that regulate the production and secretion of neuropeptides (substance P and calcitonin gene-related peptide (CGRP)). These neuropeptides stimulate tear production in lacrimal glands. Upon binding to neurokinin-1 and CGRP receptors, substance P and CGRP initiate the activation of cyclic adenosine monophosphate (cAMP) and PLC-driven intracellular signaling cascades within the lacrimal gland cells. The cAMP pathway stimulates the opening of chloride (Cl—) channels, allowing the secretion of Cl-ions into the lacrimal gland ducts. This is followed by the efflux of sodium (Na+) ions and water, creating an osmotic gradient that drives the secretion of tears. Activated PLC catalyzes the hydrolysis of phosphatidylinositol 4,5-bisphosphate (PIP2) into inositol 1,4,5-trisphosphate (IP3) and diacylglycerol (DAG). IP3 diffuses into the ER of lacrimal gland cells and binds to IP3 receptors, causing the release of Ca²⁺ ions from intracellular stores. The increase in intracellular calcium concentration activates calcium-activated chloride channels (CaCCs) present on the apical membrane of lacrimal gland cells. These channels allow the efflux of chloride ions (Cl—) from the cytoplasm into the lumen of the lacrimal gland ducts. The efflux of chloride ions creates an osmotic gradient, which drives the movement of sodium ions (Na+) and water from the interstitium into the lumen of the ducts through paracellular and transcellular pathways. Along with fluid secretion, the activation of the PLC pathway also stimulates the secretion of electrolytes into the lacrimal gland ducts. The combined effect of fluid and electrolyte secretion in the lacrimal gland results in tear production. Since TRPM8/neuropeptide-dependent activation of cAMP and PLC in lacrimal gland cells is essential for optimal tear production and proper hydration of the ocular surface, any dysfunction of the TRPM8 receptors leads to reduced tear production and causes dryness of the ocular surface.

In other embodiments, in addition to its effect on tear secretion, TRPM8 modulates the expression of genes that regulate the syntheses of tight junction proteins (occludin, claudin), adhesion molecules (E-cadherin), and actin-binding proteins (myosin light chain), maintaining the stability and integrity of the epithelial barrier on the ocular surface. Accordingly, dysfunction of the TRPM8 receptors causes an increased permeability of the ocular surface epithelium, enhanced evaporation of tears, decreased tear film stability, and dryness of the ocular surface. Also, TRPM8 dysfunction causes heightened ocular surface sensitivity, discomfort, and pain, which are the main clinical symptoms reported by DED patients.

In embodiments, these findings are in line with results obtained by other researchers in two pilot clinical trials, which revealed that topical administration of the TRPM8 agonist (cryosim-3) significantly increased basal tear production and lessened neuropathic pain in DED patients, indicating that the use of TRPM8 agonists could be considered as a potential new therapeutic approach for the treatment of DED-related ocular discomfort. Approximately half of corneal TRPM8+ neurons express the TRPV1 channel that allows the passage of calcium, sodium, and potassium ions. TRPV1 is highly expressed in corneal and conjunctival TRPM8+ sensory neurons, and in anterior eye samples and trigeminal ganglia of mice and rats with DED. An increased osmolarity of the tear film triggers a conformational change in these TRPV1 channels, leading to their opening. Massive influxes of cations cause disruption to cellular homeostasis. Accordingly, THO-dependent prolonged activation of TRPV1 channels causes tear film instability and compromises the integrity of the epithelial barrier in the eyes, exacerbating DED. Also, activation of TRPV1 in human CECs importantly contributes to the development of ocular inflammation in DED patients. TRPV1-dependent increases in the intracellular concentration of calcium, which leads to the activation of mitogen-activated protein kinase (MAPK) and transcriptional factor NF-κB, resulting in an enhanced production of inflammatory cytokines (IL-6 and IL-8). Additionally, the nociception experienced with DED is a result of TRPV1 activation on the ophthalmic branch of the corneal trigeminal nerve endings. Bearing in mind that a continuous activation of TRPV1 aggravates DED, Benitez-Del-Castillo and colleagues conducted phase I and II pilot clinical trials to test the safety and efficacy of SYL1001 (TRPV1-specific short interfering RNA) in DED patients. Topical administration of SYL1001 significantly extended tear break-up time, prevented THO, improved the disease index score of ocular surfaces, and attenuated conjunctival hyperemia in the eyes of DED patients, without causing any severe side effects.

### The Impact of Hormones, Medication, and Environmental Factors on Tear Osmolarity

In embodiments, hormones (cortisol, aldosterone, thyroid hormones, etc.), medications (antihistamines, beta-blockers, diuretics, isotretinoin, anti-cholinergics, etc.), and environmental factors all affect tear osmolarity and regulate tear-dependent eye protection. Cortisol can increase tear osmolarity by enhancing the expression of sodium channels in CECs, which can lead to an increase in the transportation of sodium ions into the tear film. Aldosterone, on the other hand, can increase the production of sodium-potassium pumps in the cornea, which can lead to an increase in the transportation of potassium ions out of the tear film. This can also lead to an increase in tear osmolarity. Thyroid hormones (thyroxine (T4) and triiodothyronine (T3)) can affect the metabolism of cells in the lacrimal gland, which can influence the production and composition of tears, affecting tear osmolarity.

The increased production of thyroid hormones in overactive thyroid glands usually results in increased tear production and decreased tear osmolarity, as the metabolism of cells in the lacrimal gland is increased. Therefore, epiphora, watery eyes, is frequently observed in patients with hyperthyroidism. On the other hand, patients with hypothyroidism, or an underactive thyroid gland, may have a decreased tear production and an increased tear osmolarity, as the metabolism of cells in the lacrimal gland is decreased. Lucius and colleagues showed that a thyroxine metabolite, 3-iodothyronamine (3T1AM), could act as a TRPM8 agonist. A significant increase in the intracellular Ca²⁺ influx was observed in 3T1AM-exposed immortalized human CECs, suggesting that 3T1AM-type analogs should be used as the leading compounds in developing drugs that would directly activate TRPM8 and reduce desiccation of the anterior ocular surface epithelia. Since THO induces pathological changes in corneas, conjunctivas, and lacrimal glands, and elicits a detrimental immune response in the eye, THO is considered an important step in the development of DED.

### THO-Related Pathological Changes in the Eyes of DED Patients

In embodiments, lid margins, preocular tear film, and conjunctival sacs display distinct tear volumes and different tear osmolarities. Decreased tear flow and environmental factors (low relative humidity, high wind speed, raised air temperature) may increase tear osmolarity; it is at its the lowest upon waking, and rises very quickly during the day, reaching baseline levels within 40 min. Tear osmolarities are usually measured using samples drawn from the lower tear meniscus in resting conditions. The average tear osmolarity in resting conditions, measured in lower tear menisci of healthy eyes in resting conditions, is 302±9.7 mOsm/L. The ocular surface, when exposed to tears with osmolarities ranging from 292.3 to 311.7 mOsm/L, is protected from any harmful effects of osmolarity. The most sensitive baseline for distinguishing normal eyes from those with mild-moderate DED is a tear osmolarity of 308 mOsm/L (measured in the lower meniscus), and the most definite cut-off is 316 mOsm/L. Tear osmolarities of up to 519 mOsm/L have been reported clinically, and the highest levels of osmolarity have been hypothesized to be at sites of tear film break-up.

The tear film is composed of three distinct (lipid, aqueous, and mucin) layers, which work together to provide lubrication, protect the ocular surface from infections, and maintain clear vision. The lipid layer reduces tear evaporation, the aqueous layer provides moisture and nutrients, and the mucin layer ensures the proper adherence and distribution of tears. Any disruption or imbalance in these layers can lead to tear film instability, which importantly contributes to THO development. Tears are drained via the nasolacrimal system and lost via evaporation. Enhanced tear evaporation disrupts the layered structure of the tear film and increases its osmolarity. Specifically, tear osmolarity will double if evaporation reduces the aqueous layer of tear film to half of its initial thickness, provided that any tear solutes, including salts, are not lost from the aqueous layer (for example, via diffusion out of the region).

In other embodiments, enhanced evaporation reduces the tear film thickness, disrupting its normal structure and making it more susceptible to break-up. Evaporation-driven tear film rupture is observed using fluorescence imaging as black circular spots, linear streaks, or irregular pools on a yellow-green fluorescence background. The tear film is refreshed by blinking, which, in people with healthy eyes, occurs with a frequency of 10 to 30 times per minute. When blinking is slowed, tear film break-ups appear within 15 to 40 s, in individuals with healthy eyes. However, tear film break-up regions (“black spots/streaks”) have been observed in the eyes of DED patients every 2-3 s. Importantly, these “black spots/streaks” arose in areas where the aqueous layer of the tear film was entirely ruptured down to the underlying mucin/corneal interface, leaving the underlying epithelial cells completely unprotected. Since osmolarity in the tear film break-up regions of DED patients can be dramatically high (between 800 and 900 mOsM), THO is recognized as a major cause of ocular surface damage in DED patients. The most damaging THO-dependent pathological changes mainly develop in local salinity spots, which arise in the black spots of tear breakup.

Since the process of osmoregulation, which guard the ocular surface epithelia and maintain normal vision, is affected when the osmolarity of the tears is greater than that of the epithelial cells, THO is considered as an initial, important step in the development, progression, and aggravation of DED. Hypertonicity induces activation of the TRPV1 signaling pathway in CECs, resulting in the activation of nuclear factor kappa B (NF-κB). By triggering NF-κB signaling, THO induces apoptotic cell death in CECs and disrupts the integrity of the corneal epithelial barrier, leading to increased permeability and susceptibility to infection.

In other embodiments, it is observed that hyperosmotic environments caused the apoptosis of corneal and conjunctival epithelial cells by inducing dysfunction in the ER. The ER is cellular organelle that contains chaperones and folding enzymes, which facilitate the assembly of newly synthesized proteins, preventing their misfolding and aggregation. A hyperosmotic microenvironment affects the transportation of secretory content in the ER and attenuates its capacity to fold proteins. The resulting accumulation of unfolded proteins activates a cascade of stress-related signaling, referred to as the unfolded protein response (UPR), which transiently attenuates translation and increases protein folding in ERs of stressed epithelial cells. An increased UPR activity, induced by THO, enhances the synthesis of transcription factor X-box binding protein 1 (XBP1), which directs the expression of chaperones and folding enzymes to restore homeostasis. If THO induces severe ER stress that cannot be attenuated by XBP1s, the UPR increases the activity of DNA damage-inducible transcript 3 protein (DDIT3), initiating apoptosis and the removal of stressed corneal and conjunctival epithelial cells. THO-dependent induction of ER-stress-associated programmed death of these cells results in the disruption of the ocular surface epithelial barrier, paving the way for the entrance of invading pathogens in the eyes of DED patients.

In embodiments, THO-induced changes in the phenotype and function of CECs. Apoptotic cell death is observed in THO-exposed CECs. THO induces stress in the ER and promotes mitochondrial damage by enhancing Fas- and TRAIL-dependent activation of caspase-3, capsase-8, and caspase-9, which results in apoptosis of THO-exposed CECs. Additionally, THO promotes an increased expression of E and P selectins and induces an enhanced production of inflammatory cytokines (TNF-α, IL-1β, IL-6) in CECs, which results in a massive influx of circulating immune cells in inflamed eyes of DED patients, resulting in the aggravation of ongoing inflammation.

Additionally, THO-dependent activation of NF-κB in CECs is responsible for a massive influx of circulating leukocytes in inflamed eyes of DED patients. THO-exposed CECs express high levels of adhesion molecules and massively produce pro-inflammatory cytokines (interleukin (IL)-1β, and tumor necrosis factor alpha (TNF-α)) which attract circulating immune cells to the inflamed corneas and lacrimal glands of DED patients. Upon binding to their receptors, IL-1β and TNF-α induce an increased expression of E and P selectins on the membrane of endothelial cells, enabling the adhesion, recruitment, and influx of circulating leukocytes in inflamed eyes. Additionally, IL-1β and TNF-α stimulate the enhanced production of inflammatory cytokines in eye-infiltrated immune cells, creating a “positive inflammatory loop” that leads to the progression of DED. IL-1β and TNF-α activate several kinases (mixed lineage kinase (MLK)-2/-3, transforming growth factor β-activated kinase 1 (TAK1), mitogen-activated protein kinase (MKK)-1, apoptosis signal-regulating kinase 1 (ASK1), MKK7, and Jun N-terminal kinase (JNK)) in eye-infiltrated dendritic cells (DCs). These kinases phosphorylate and activate transcriptional factors c-Jun and ATF2. Activated c-Jun and ATF2 translocate to the nucleus to induce an enhanced expression of pro-Th1 (IL-12) and pro-Th17 cytokines (IL-1β, IL-6, IL-23) in DC. Through the production of these inflammatory cytokines, DCs activate Th1- and Th17-specific transcriptional factors (T-box transcription factor TBX21 (T-bet), and RAR-related orphan receptor gamma (ROR-γ)) in naïve T cells, inducing their differentiation in effector, inflammatory, Th1, and Th17 lymphocytes. Th1 cell-derived interferon gamma (IFN-γ) and Th17 cell-sourced IL-17 and IL-22 activate macrophages and neutrophils in the inflamed eyes of DED patients, crucially contributing to the aggravation of DED.

### THO Stimulation of T Cell-Driven Detrimental Immune Responses in the Eyes of DED Patients

In embodiments, THO-dependent apoptosis of CECs disrupts the integrity of the mucosal barrier of the ocular surface, enabling a massive influx of invading pathogens. Antigens that bypass the THO-injured epithelial barrier are captured by skillful antigen-presenting DCs that reside in the corneal epithelium. THO induces an increased activation and maturation of DCs and promotes their recruitment in inflamed corneas and conjunctivas by enhancing the expression of adhesion molecules on epithelial and endothelial cells. Accordingly, one study observed a significantly increased number of activated DCs in the ocular surfaces of THO mice compared to healthy controls.

Detrimental immune response in the eyes of DED patients. Increased expressions of MHC proteins and co-stimulatory molecules were noticed in eye-infiltrated DCs of DED patients. DCs of DED patients have increased capacities for the production of inflammatory, pro-Th1 (IL-12), and pro-Th17 cytokines (IL-1β, IL-6, IL-12, IL-23), crucially contributing to the generation of detrimental Th1 and Th17 cell-driven immune responses. Accordingly, an increased number of IFN-γ-producing Th1, NK, and NKT cells and an increased number of IL-17- and IL-22-producing Th17 cells were observed in the eyes of DED patients. Additionally, THO promotes NETosis in eye-infiltrated neutrophils, importantly contributing to the progression of DED.

In other embodiments, upon the engulfment of invading pathogens, activated DCs process their antigens and present them to naïve CD4+T lymphocytes in regional lymph nodes. In one study, the DCs isolated from eye-draining lymph nodes of THO mice had better antigen-presenting properties than the DCs that infiltrated regional lymph nodes of control mice. Higher expressions of molecules that are responsible for DC-dependent activation of naïve T cells (co-stimulatory and major histocompatibility complex (MHC) proteins) were observed on the membranes of THO-primed corneal and conjunctival DCs, indicating that a hyperosmolar microenvironment promotes efficient antigen presentation and the optimal activation of T cells. Accordingly, a higher number of activated CD4+T cells were observed in the eye-draining lymph nodes of THO mice than in control animals.

THO promotes MKK7- and JNK-dependent activation of c-Jun and activating transcription factor 2 (ATF2) in corneal and conjunctival DCs. Upon the activation of c-Jun and ATF2, DCs enhance the production of inflammatory cytokines IL-12, IL-1β, IL-6, and IL-23. While DC-derived IL-12 triggers Th1-driven eye inflammation, DC-sourced IL-1β, IL-6, and IL-23 are responsible for the generation of Th17 cell-dependent injury in meibomian and lacrimal glands. Through the production of IL-12, DCs activate signal transducers and activators of (STAT)-1 and T-bet transcriptional factors in naïve CD4+ T lymphocytes, inducing their differentiation in effector IFN-producing Th1 cells. Similarly, by activating STAT-3 and RAR-related orphan receptor gamma T (RORγT) transcriptional factors in naïve CD4+ T cells, DC-derived IL-1β, IL-6, and IL-23 induce the generation and expansion of inflammatory CD4+Th17 cells.

In embodiments, CD4+Th1 lymphocytes, in an IFN-γ dependent manner, and CD4+Th17 lymphocytes, in IL-17- and IL-22-dependent ways, induce the generation of inflammatory M1 and N1 phenotypes in the eye-infiltrating macrophages and neutrophils of DED patients. These cells are valuable sources of inflammatory mediators, which contribute to the reduced expression of mucins, apoptotic death of corneal and conjunctival epithelial cells, and to the loss of goblet cells in inflamed eyes of DED patients. Additionally, M1 macrophages and N1 neutrophils produce Th1 and Th17 cell-attracting chemokines (C—X—C motif ligand (CXCL)-9/-10, C—C motif ligand (CCL)-20), enabling massive influxes of CD4+ and CD8+ Th1 and Th17 cells in inflamed eyes of DED patients.

Th1 and Th17 lymphocytes, through the production of inflammatory cytokines (IFN-γ and IL-17), have been shown to induce disruption of the corneal epithelial barrier, trigger injury of the meibomian glands, and reduce tear production in the eyes of DED mice. Also, antibody-dependent neutralization or genetic deletion of IFN-γ and IL-17 significantly attenuated eye inflammation and almost completely prevented corneal epithelial barrier dysfunction in the eyes of DED mice, confirming the crucially important pathogenic role of these inflammatory cytokines in the pathogenesis of DED. Th17 cell-derived IL-17 increases the expression of MMP-3 and MMP-9 which, in turn, induces disruption of the corneal epithelial barrier. Th1 cell-sourced IFN-γ activates both extrinsic and intrinsic apoptotic pathways in CECs. The extrinsic or “death receptor” route is mediated by Fas ligand (FasL)- and TNF-related apoptosis-inducing ligand (TRAIL)-dependent activation of caspase-8, and the intrinsic or “mitochondrial” route is mediated by caspase-9. In further research findings, while exogenous administration of IFN-γ significantly increased caspase-3, -8, and -9-dependent apoptosis in conjunctival epithelial cells, pharmacological inhibition or genetic deletion of IFN-γ remarkably alleviated immune cell-driven injury of conjunctival epithelial and goblet cells in the eyes of DED mice. Similar results have been observed in DED patients; IL-17-dependent corneal barrier disturbance was linked to an increased risk of corneal ulcers, corneal haze, and vision loss, while increased levels of IFN-γ was related to conjunctival epithelial squamous metaplasia, with progressive goblet cell loss.

In other embodiments, passive transfer experiments are used to investigate whether detrimental Th1/Th17 cell-driven immune responses initiated by the disruptive effect of THO was sufficient to induce DED under mild desiccating stress. Recipient mice were randomly divided in the two experimental groups to receive either T cells from the spleens and lymph nodes of THO mice or control (healthy) mice. Upon the passive transfer of T lymphocytes, recipient mice were exposed to mild desiccating stress (forced airflow without scopolamine). CD4+ and CD8+ T cells from THO mice homed more efficiently to lymph nodes than their control counterparts, and induced strong and potent detrimental immune responses that caused DED-related clinical symptoms in the recipient animals. A gradual reduction in tear production and damage and an increased irregularity of the corneal surface were observed in the eyes of the experimental animals that received the T cells from THO mice. These pathological changes were not observed in the animals that were injected with T cells from the control group, indicating the important role of THO in the priming of T cell-driven eye inflammation.

In embodiments, it has to be noted that the passive transfer of THO-primed T cells managed to induce DED in recipient animals only upon the depletion of immunosuppressive FoxP3-expressing CD4+T regulatory cells (Tregs). Contrary to the inflammatory IFN-γ- and IL-17-producing Th1 and Th17 cells, Tregs produce immuno-modulatory factors (retinoic acid, IL-10, and transforming growth factor beta (TGF-β)), which attenuate the maturation and antigen-presenting properties of ocular DCs, prevent the generation of CD4+Th1 and Th17 lymphocytes in eye-draining lymph nodes, and inhibit the production of inflammatory cytokines (TNF-α and IL-1β) and Th1- and Th17-attracting chemokines (CXCL9, CXCL10, CCL-20) in N1 neutrophils and M1 macrophages, crucially contributing to the creation of anti-inflammatory microenvironments in the eyes of DED patients. THO impairs immune homeostasis at the ocular surface by altering the ratio between inflammatory and regulatory T cells. THO favors the generation of pro-inflammatory (immunogenic), instead of immunosuppressive (tolerogenic), phenotypes in eye-infiltrated DCs. While immunogenic DCs induce eye inflammation by promoting the activation of Th1 and Th17 cells, tolerogenic DCs create immunosuppressive microenvironments in the inflamed eyes of DED patients by inducing the expansion of Tregs. A significantly reduced presence of immunosuppressive Tregs and increased numbers of activated CD69-expressing T lymphocytes, memory (CD62L^{lo} CD44^{hi}) CD4+ T cells, and CD103-expressing CD8+ cytotoxic T cells have been observed in the draining lymph nodes of THO mice compared to healthy controls, indicating that THO-dependent modulation of DC phenotypes and functions altered the cellular make-up of eye-draining lymph nodes and promoted the progression of T cell-driven eye inflammation.

### Modulation of Eye-Infiltrated Neutrophils and Natural Killer Cells Via THO

In embodiments, several lines of evidence have demonstrated that, in addition to macrophages, DCs, and T cells, THO modulates the phenotype and function of eye-infiltrated neutrophils and natural killer (NK) cells. NETosis is a process in which neutrophils release their DNA and antimicrobial substances to trap and kill invading pathogens in the eyes of DED patients. However, excessive NETosis can cause damage to the ocular surface and exacerbate ongoing inflammation, leading to further tissue damage and worsening of DED-related symptoms. Tibrewal and colleagues showed that THO promoted NETosis in inflamed eyes, which leads to the aggravation of DED, and that neutrophils exposed to hyperosmolar conditions had morphological alterations (bigger and more rounded nuclei) and an increased capacity for NET production. To validate this influence of hyperosmolar circumstances on NET production, Tibrewal and colleagues restored iso-osmolar conditions, and, subsequently, NET formation was significantly reduced. Furthermore, the inclusion of NET formation inhibitors (such as staurosporine and anti-2 integrin) resulted in a reduction in the quantity of NETs under hyperosmolar conditions.

In embodiments, because they are an early source of IFN-γ during the induction phase of experimental dry eye illnesses, natural killer (NK) and natural killer T (NKT) cells have been implicated in the immunopathogenesis of DED. THO induces oxidative damage in CECs, which manifests with an increased expression of damage-associated molecular patters (DAMPs) and oxidative damage markers, and with increased productions of heme oxygenase-1 (HMOX1), cyclooxygenase-2 (COX2), and heat shock proteins (HSPs). NK and NKT cells recognize DAMPs and oxidative stress-related proteins that are expressed on the membranes of THO-exposed CECs. This recognition can trigger the activation of NK and NKT cells, which results in a massive production of inflammatory cytokines, including IFN-γ and IL-17. NK and NKT cell-sourced IFN-γ enhances the expression of major histocompatibility complex (MHC) class II molecules and promotes the synthesis of IL-12 in DCs, enabling the generation of optimal Th1 cell-driven immune responses in the eyes of DED patients. Similarly, an increased production of the Th17 cell-attracting chemokine (CCL20) and enhanced synthesis of pro-Th17 cytokines (IL-1β, IL-6, and IL-23) were observed in NK and NKT cell-primed DCs. The alleviation of THO, as well as the depletion of NK/NKT cells, significantly reduced the presence of activated DCs in the inflamed eyes of DED mice, alleviated their antigen-presenting properties, attenuated Th1 and Th17 cell-driven eye inflammation, down-regulated MMP-3 and MMP-9 expression, and reduced apoptosis of their corneal and conjunctival epithelial cells, confirming the important pathogenic role of THO in NK and NKT cell-driven inflammation in the eyes of DED patients.

THO initiates and aggravates DED by: (i) inducing destruction of corneal and conjunctival epithelial barriers, (ii) enhancing Th1 and Th17 cell-driven immune responses, (iii) promoting NET formation, and (iv) stimulating NK and NKT cell-dependent inflammation in the eyes of DED patients. Since THO plays an important pathogenic role in the development and progression of DED, hypo-osmotic eye drops can be used as a treatment for the alleviation of THO and DED. Hypo-osmotic eye drops contain a lower concentration of salt and other solutes compared to the tears on the ocular surface, which can help to reduce THO and improve symptoms of dryness, burning, and irritation in the eyes of DED patients. When hypo-osmotic eye drops are instilled into the eye, they may help to restore the natural osmotic balance of the ocular surface, which can improve tear film function and decrease inflammation. Hypo-osmotic eye drops may also decrease THO-induced damage of corneal and conjunctival epithelial barriers. However, it is important to note that hypo-osmotic eye drops may not be effective for all DED patients and that artificial tears, anti-inflammatory agents, or immunomodulatory drugs may be necessary to manage the underlying inflammation and improve their symptoms. In line with these findings, it has to be noted that human CECs have the capacity to regulate their volume upon exposition to anisosmotic stress. Importantly, in the case of hypertonic stress, the time delay for the onset of regulatory volume increases was longer than that for regulatory volume decreases. Cell swelling developed within 30 s and then decreased over time. In other embodiments, it is disclosed that small interfering RNA (siRNA)-dependent silencing of TRPM7 significantly reduce the rate of cell volume recovery after osmotic swelling, indicating a crucially important role of TRPM7 in volume regulation of human epithelial cells. Several lines of evidence have demonstrated that the expression of TRPV4 in human CECs was also important for maintaining the corneal epithelial barrier integrity during exposure to hypotonic stress. When tear osmolarity decreases, the TRPV4 channels are activated, allowing the influx of Ca²⁺ ions into the cells. An increase in the intracellular concentration of calcium triggers a cascade of calcium-dependent signaling, which results in enhanced syntheses of substance P and CGRP. These neuropeptides subsequently stimulate the lacrimal gland to produce tears and restore tear osmolarity, preventing dryness-dependent damage to the ocular surface.

### Therapeutic Potential of Hypo-Osmotic Eye Drops in DED Treatment

In embodiments, THO induces dehydration and consequent injury of CECs. Hypo-osmotic eye drops contain a lower concentration of salt and other solutes compared to the tears on the ocular surface. Accordingly, hypo-osmotic eye drops restore the natural osmotic balance of the ocular surface, improve tear film function, and attenuate THO-induced damage of the corneal epithelial barrier.

THO-induced pathological changes could also occur as a result of inadequate meibum consistency. The meibum of patients with MGD-related DED has a toothpaste-like consistency and is not able to maintain stability and integrity of the tear film. As a result, the tear film becomes more prone to evaporation, leading to an increased concentration of solutes in the remaining tears, which finally results in the development of THO-dependent pathological changes. Accordingly, the use of intense pulsed light treatment and omega-3 fatty acids, phospholipids, triglycerides, and anti-oxidants (vitamin C and E)-enriched supplements, which manage to improve meibum consistency, stabilize the tear film, and attenuate THO-dependent injury and inflammation, should be considered as a supportive therapeutic approach for the treatment of DED patients.

In other embodiments, since THO induces apoptosis of corneal and conjunctival epithelial cells by triggering the activation of NF-κB signaling pathways in CECs, a combined use of NF-κB inhibitors and hypo-osmotic ophthalmic solutions could be used for the eradication of DED-related signs and symptoms in the injured and inflamed eyes of DED patients. Also, bearing in mind the important pathogenic role of NETosis, NK/NKT cells, and Th1/Th17 lymphocytes in the pathogenesis of THO-dependent eye inflammation, future experimental and clinical studies should investigate whether a combined administration of hypo-osmotic eye drops with immunosuppressive and anti-inflammatory drugs would have beneficial effects in the DED treatment.

Finally, it has to be noted that blinking and tear drainage almost immediately voids the artificial tears. Therefore, the expectation that hypo-osmotic eye drops will manage to completely reduce DED-related symptoms must be considered with caution. Several strategies could be used to ensure longer-lasting effects of hypo-osmotic eye drops and prevent them from being voided by blinking and via tear drainage. An addition of ointment-based supplements to hypo-osmotic eye drops could provide longer-lasting lubrication, reducing the risk of their rapid removal by blinking. Also, since preservatives in artificial tears sometimes cause irritation and increase the need for blinking, the use of preservative-free hypo-osmotic eye drops will reduce this risk and will provide longer-lasting effects. The use of punctual plugs, small devices which are inserted into the tear ducts to block tear drainage, could also provide hypo-osmotic eye drops with longer-lasting effects. In line with all of these facts, although experimental and clinical studies have provided evidence of the crucially important pathogenic role of THO in the development and progression of DED, the therapeutic efficacy of hypo-osmotic eye drops should be confirmed in large randomized clinical trials before these drugs can be widely offered as new remedies for DED treatment.

Compounds and/or Formulations Containing De-Cellularized Human Amniotic Fluid

In at least one embodiment of the disclosure, d-MAPPS pharmaceutical compositions (e.g., d-MAPPS solutions) include sterile de-cellularized human amniotic fluid (D-HAF), either in fluid form or solid form, for example, lyophilized powder, alone or in combination with appropriate excipients. Other active agents can be included. D-HAF contains over 300 human growth factors. D-HAF is devoid of amniotic stem cells and elements of micronized membrane or chorion particles.

Amniotic fluid (“AF”) contains nutrients and growth factors that facilitate fetal growth, provides mechanical cushioning and antimicrobial effectors that protect the fetus, and allows assessment of fetal maturity and disease. AF typically contains mixtures of growth factors, pro-inflammatory cytokines and anti-inflammatory cytokines, as well as a variety of macromolecules including carbohydrates, proteins and peptides such as enzymes and hormones, lipids, lactate, pyruvate, and electrolytes.

In some embodiments, the raw fluid directly collected from the source is not heat-treated, chemical-treated, or fractionated to produce the disclosed formulations. In some embodiments, the formulation retains more than 50%, more than 60%, more than 70%, more than 80%, or preferably more than 90%, of the total amniotic factors present in the raw fluid. In some embodiments, the formulations are not diluted with any additional solution for storage. In some embodiments, the formulations are diluted prior to application to the eyes. In some embodiments, the formulations are not concentrated relative to the raw fluid.

In some embodiments, the d-MAPPS composition (e.g., one or more d-MAPPS solutions) includes a diluted sterile de-cellularized human amniotic fluid (D-HAF), which preferably has not been heat treated, typically administered using a standard eye dropper apparatus. D-HAF contains over 300 human growth factors. D-HAF is devoid of cells, including amniotic stem cells, and elements of micronized membrane or chorion particles. The purified fluid is sterilized without the use of harsh terminal irradiation, e-beam or Ethylene Oxide (EO). In at least a further embodiment, the process includes separating the cells from the AF using centrifugation and utilizing a series of filtration devices to remove all remaining cells and bioburden. Each lot is tested for bioburden and is certified sterile to contain <1 harmful organisms.

Generally, methods of preparing sterile de-cellularized amniotic fluids involve a series of centrifugation and filtration steps. Preferred methods of preparing sterile de-cellularized amniotic fluid are described in detail in U.S. application Ser. No. 15/053,497.

### Method of Preparation

In some embodiments, D-HAF is prepared from sterile human amniotic fluid obtained from a woman, removing cells, large particles and other undissolvables are removed, preferably by high speed centrifugation to obtain clarified amniotic fluid, the clarified amniotic fluid filtered through filters having a pore size of about 5 μm to about 10 μm to obtain a micron filtrate, filtering the micron filtrate through filters with a pore size of about 1.0 μm to obtain a second filtrate, filtering the filtrate through submicron filters with the pore size of 0.45 μm or/and 0.2 μm to obtain the sterilely filtered amniotic fluid.

In some embodiments, a collection procedure is performed in a sterile operating room environment during an elective C-section. Typically, the woman is undergoing a pre-Caesarian surgical procedure. The steps of obtaining the sterile human amniotic fluid includes the steps of turning on a ultrasound device to provide guidance for the process of obtaining human fluid from the woman, inserting a blunt tip needle into the amniotic sac of the woman, attaching the blunt tip needle to a three-way stopcock, connecting a Luer lock syringe to the three-way stopcock, connecting a first end of a length of sterile tubing with the three-way stopcock, and collecting sterilely the amniotic fluid through the blunt tip needle and sterile tubing into a collection container.

In this embodiment, the sterile collection container includes a pump with a suction device. The suction device is a low suction device or a spring loaded low suction device. The suction device is fluidly connected to an internal balloon. This embodiment further includes manually pumping up the internal balloon in the sterile collection container using the low suction device to allow a low-level suction and collection of the amniotic fluid.

In some embodiments, the step of removing cells, large particles and other undissolvables from the human amniotic fluid includes a first step of centrifuging or depth filtering the human amniotic fluid. In some embodiments, the human amniotic fluid is centrifuged at about 5,000 rpm to about 10,000 rpm for about 30 minutes to about 60 minutes. In this embodiment, filters of about 5 μm to about 10 μm are used for the first step. These can be cellulose ester filters, glass fiber filters, nylon capsule filters or nylon cartridge filters. The filters with the pore size of 1.0 μm are capsule filters or cartridge filters. The filters with the pore size of 1.0 μm are poly ether sulfone, poly vinylidene fluoride or cellulose acetate membrane filters. The filters with the pore size of 0.45 μm or 0.2 μm are capsule filters or cartridge filters. The filters with the pore size of 0.45 μm or 0.2 μm are poly ether sulfone membrane filters, poly vinylidene fluoride or cellulose acetate membrane filters.

The sterilely filtered human amniotic fluid contains growth factors including human growth hormone, transforming growth factor beta 1, vascular endothelial growth factor, epidermal growth factor, transforming growth factor beta 3, and growth differentiation factor 11 or combinations thereof.

In some embodiments, the process of obtaining the sterile amniotic fluid further includes the step of lyophilizing the sterile amniotic fluid to obtain a lyophilisate. The lyophilisate can be further sterilized by e-beam irradiation or gamma ray irradiation to reinforce the sterility.

Tools to obtain sterilely filtered human amniotic fluid from a woman, include a three-way stopcock, a sterile blunt tip needle aseptically attached to the three-way stopcock, a Luer lock syringe aseptically connected to the three-way stopcock, a sterile tubing aseptically connected to the three-way stopcock, a collection container or a collection container including a pump with suction device connected with the sterile tubing, a set of filters having the pore size of about 5 μm to about 10 μm, a set of capsule or cartridge filters having the pore size of about 1 μm, a set of capsule or cartridge filters having the pore size of about 0.45 μm or 0.2 μm, a set of sterile syringes or vials to store the sterile filtered amniotic fluid and operating instructions on using the kit to obtain sterilely filtered human amniotic fluid. The filters having the pore size of from about 5 μm to about 10 μm and the capsule or cartridge filters are made from cellulose ester, glass fiber or nylon. The sterile collection container may include a pump with a suction device. The suction device may be a low suction device or spring loaded low suction device. In another aspect the suction device may be fluidly connected to an internal balloon. Further to this aspect the method includes manually pumping up the internal balloon in the sterile collection container using the low suction device to allow a low-level suction and collection of the amniotic fluid. In yet another aspect the sterile collection container may include an inlet. Further to this particular aspect the method includes connecting a second end of the tubing to the inlet of the sterile collection container. The sterile collection container may include a vent having a cap.

In some embodiments, utilizing the incision site immediately prior to performing the C-section and with ultrasound guidance to protect the fetus and mother provides a minimal or no risk environment for collection.

Collection is achieved via a low level suction established within a collection container and/or via gravity. Typically, after high speed centrifugation, filtration with 5 to 10 μm filters (low protein binding filter) is used to complete the removal of cells and large particles. Submicron filtration is then conducted with 1 μm and 0.45 μm or/and 0.2 μm filters (low protein binding filter), two in a series connection, to remove gross contaminates. Under this condition, soluble growth factors will pass through this filter to achieve a semi-sterile condition, very low bioburden counts. If under a strict aseptic operation condition, a 10⁻³ sterility assurance level is achieved. A 10⁻⁶ sterility assurance level can be achieved by submicron filtration with a 0.22 μm filter (low protein binding filter) at the end and sterile packaging to achieve a sterile product. One can monitor the filtrate after each filtration step to determine which components are removed and then to determine which process to use to achieve the desirable product.

One may use membrane filters including or made of hydrophilic polyethersulphone (PES) to filter protein solutions. Filter disks for small volumes and different sizes of cartridges for larger volumes such as 1 liter and more are used. Hydrophobic membranes like PTFE which are designed for liquids devoid of proteins should not be used. Start with centrifugation at 5000 to 8000 rpm for at least 30 minutes. Next, the supernatant is filtered with a prefilter to remove residual protein aggregates and precipitates in suspension (AP20 can be used). If one directly uses a 0.6/0.2 μm filter, after prefiltration, one may experience slow filtration rates and the flow may stop too quickly. It may be desirable to make intermediate filtration steps using 1.2 μm and 0.8 μm membranes. Typically, a final filtration through 0.2 μm is necessary to get the best sterility assurance level and produce a sterile amniotic fluid for injections. The final filtrate can be stored in frozen condition at about −20° C. to about −80° C. for long term storage. In addition, the sterilely filtered amniotic fluid may be distributed in vials equipped with special rubber stoppers for sterile lyophilization.

The sterile amniotic fluid can be lyophilized to yield a lyophilisate. The sterilely filtered amniotic fluid may be distributed in vials equipped with special rubber stoppers for sterile lyophilisation. The lyophilisation is carried out in a sterile environment. The rubber stoppers on the vials are then automatically pushed down in the freeze dryer to definitively close them. Finally an aluminum cap is sealed on each vial to protect its sterile content. In such a lyophilized state, the amniotic fluid may be stored at +4° C. or room temperature for at least one year without decrease of its biological activity. The lyophilisate can be irradiated by e-beam irradiation or gamma ray irradiation to insure the sterility. For its medical use, the sterile amniotic fluid may be reconstituted by adding the initial volume of sterile water to the powder in order to restore a transparent and homogeneous physiological liquid.

### Sources of Amniotic Fluid

In some embodiments, d-MAPPS compositions (e.g., d-MAPPS solutions) that include amniotic fluid are prepared, at least in part, from sterile human amniotic fluid obtained from a pregnant woman.

Suitable sources, e.g. of human AF, include AF that is obtained from patients who are undergoing amniocentesis, patients who are undergoing a Caesarean section delivery, and patients undergoing normal delivery using a specially designed receptacle to collect the fluid after rupture of membranes.

D-HAF, and/or d-MAPPS compositions and/or d-MAPPS solutions containing D-HAF, can be stored for long periods of time, allowing for a broad range of application methods, including distribution and storage as aerosols, solutions, powders, etc. In some embodiments, d-MAPPS compositions and/or d-MAPPS solutions containing D-HAF is refrigerated at about 1° C. to about 10° C. for long-term storage. In a further embodiment, d-MAPPS compositions and/or d-MAPPS solutions containing D-HAF is refrigerated at 4° C. for up to 12 months and more. Preferably, the long-term storage does not reduce the quantity of the total soluble proteins or factors present in the D-HAF. For some embodiments, the total soluble proteins retained after long-term storage in refrigerated conditions is about 10%, 20%, 30%, 40%, 50%, 60%, 70%, 80%, or 90% of the fresh D-HAF.

D-HAF, and/or d-MAPPS compositions and/or d-MAPPS solutions containing D-HAF, can be supplied as a clear one-part solution in a suitable container for storage at 4° C., or for storage at −20° C., or at −80° C. For example, liquid formulations in prefilled aliquots can be suitable for storage at 1-5° C., or for storage at −20° C., or at −80° C. The liquid formulation can be suitable for topical application in a nebulizer or a spray. In other embodiments, the fluid can be supplied as a kit that can be stored at 4° C., at −20° C., or at −80° C. until needed.

In some embodiments, D-HAF, and/or d-MAPPS compositions and/or d-MAPPS solutions containing D-HAF, use a final filtration through 0.2 μm to produce a sterile amniotic fluid without any irradiation. In some embodiments, D-HAF, and/or d-MAPPS compositions and/or d-MAPPS solutions containing D-HAF, have a 10⁻⁶ sterility assurance level without irradiation. In other embodiments, lyophilisate derived from amniotic fluid through lyophilisation may be irradiated by e-beam irradiation or gamma ray irradiation to add another guarantee for the final sterility of the powder.

### Growth Factors, Cytokines, and Other Molecules

Growth factors and their receptors control a wide range of biological functions, regulating cellular proliferation, survival, migration and differentiation. Growth factors found in AF play a critical role in fetal growth and development.

A non-limiting list of growth factors that have been identified in AF includes factors such as epidermal growth factor (EGF), insulin-like growth factor I (IGF-I), vascular endothelial growth factor A (VEGF-A), tumor necrosis factor A (TNF-α), hepatocyte growth factor (HGF), fibroblast growth factor 7 (FGF7), matrix metallopeptidase (MMP-9), granulocyte-colony stimulating factor (GCSF), matrix metalloproteinase-7 (MMP-7), matrix metalloproteinase-13 (MMP-13), transforming growth factor alpha (TGF-α), transforming growth factor beta (TGF-β), fibroblast growth factor 4 (FGF-4), endocrine gland-derived vascular endothelial growth factor (EG-VEGF), interleukin 8 (IL-8), fibroblast growth factor 21 (FGF-21), angiopoietin-2 (ANG-2), Glial cell-derived neurotrophic factor (GDNF), fibroblast growth factor 19 (FGF-19), TIMP metallopeptidase inhibitor 2 (TIMP-2), angiopoietin-1 (ANG-1), transforming growth factor beta 1 (TGFβ1), macrophage colony-stimulating factor (M-CSF), angiotensinogen, platelet derived growth factor-AA (PDGF-AA), and stem cell factor (SCF).

Epidermal growth factor (EGF) is a small polypeptide hormone with mitogenic properties in vivo and in vitro. EGF elicits biologic responses by binding to a cell surface receptor which is a transmembrane glycoprotein containing a cytoplasmic protein tyrosine kinase. EGF responses are mediated by ligand binding and activation of this intrinsic protein kinase. The receptor can be phosphorylated by other protein kinases, and this may regulate receptor function. Stimulation of the receptor tyrosine kinase activity by ligand binding must regulate the activity of an as yet undefined molecule(s) responsible for transmitting a mitogenic signal to the nucleus (Todderud G., et al., Biofactors. 1989, 2(1): 11-5).

Vascular endothelial growth factor (VEGF), also known as vascular permeability factor (VPF), was originally described as an endothelial cell-specific mitogen. VEGF is produced by many cell types including tumor cells, macrophages, platelets, keratinocytes, and renal mesangial cells. The activities of VEGF are not limited to the vascular system; VEGF plays a role in normal physiological functions such as bone formation, hematopoiesis, wound healing, and development (Duffy A. M., et al., In: Madame Curie Bioscience Database [Internet]. Austin (TX): Landes Bioscience (2000)).

TGF-α has a structure similar to EGF and binds to the same receptor. The amnion cells of the umbilical cord express EGF, TGF-α, and the functional EGF/TGF-α receptor, suggesting the possibility of a regulating role of the amnion in fetal growth and development. EGF and TGF-α have also been shown to stimulate the production of surfactant components.

TGFβ1 is believed to induce terminal differentiation of intestinal epithelial cells and to accelerate the rate of healing of intestinal wounds by stimulating cell migration. TGFβ1 may also stimulate IgA production. VEGF-A is a signal protein that stimulates vasculogenesis and angiogenesis (Hoeben Am, et al., Pharmacol Rev. 2004, 56:549-580).

Transforming growth factor-beta (TGF-β) is a multifunctional peptide that controls proliferation, differentiation, and other functions in many cell types. Many cells synthesize TGF-B and essentially all of them have specific receptors for this peptide. TGF-β regulates the actions of many other peptide growth factors and determines a positive or negative direction of their effects (Sporn M. B., et al., Science 1986, 233(4763) 532-534).

Hepatocyte growth factor (HGF), the ligand for the receptor tyrosine kinase encoded by the c-Met proto-oncogene, is a multidomain protein structurally related to the pro-enzyme plasminogen and with major roles in development, tissue regeneration and cancer. A recent study showed its immunomodulation potential of amniotic fluid stem cells (Maraldi T., et al., Stem Cells Transl. Med., 4(6): 539-47 (2015)).

Fibroblast growth factors (FGFs) that signal through FGF receptors (FGFRs) regulate a broad spectrum of biological functions, including cellular proliferation, survival, migration, and differentiation. The FGF signal pathways are the RAS/MAP kinase pathway, PI3 kinase/AKT pathway, and PLCγ pathway, among which the RAS/MAP kinase pathway is known to be predominant. Several studies have recently implicated the in vitro biological functions of FGFs for tissue regeneration. Many current applications of FGF are in regeneration of tissues, including skin, blood vessel, muscle, adipose, tendon/ligament, cartilage, bone, tooth, and nerve tissues (Yun Y. R., et al., J. Tissue Eng. 2010: 1(1)).

Matrix metalloproteinases (MMPs), also called matrixins, function in the extracellular environment of cells and degrade both matrix and non-matrix proteins. They play central roles in morphogenesis, wound healing, tissue repair and remodeling in response to injury, e.g., after myocardial infarction, and in progression of diseases such as atheroma, arthritis, cancer and chronic tissue ulcers. They are multi-domain proteins and their activities are regulated by tissue inhibitors of metalloproteinases (TIMPs) (Nagase H., et al., Cardiovascular Research, European Society of Cardiology, 562-573 (2006)).

Amniotic fluid also contains many pro- and anti-inflammatory cytokines. Pro- and anti-inflammatory cytokines play important immunoregulatory roles. Inflammation is characterized by interplay between pro- and anti-inflammatory cytokines. Cytokines are commonly classified in one or the other category: interleukin-1 (IL-1), tumor necrosis factor (TNF), gamma-interferon (IFN-γ), IL-12, IL-18, and granulocyte-macrophage colony stimulating factor are well characterized as pro-inflammatory cytokines, whereas IL4, IL-10, IL-13, IFN-α and TGF-β are recognized as anti-inflammatory cytokines.

Exemplary pro-inflammatory cytokines include Eotaxin-2 (CCL24), interleukin 6 (IL-6), pulmonary and activation-regulated chemokine PARC or chemokine (C—C motif) ligand 18 (CCL18), total GRO which consisted of three subunits GROα/CXCL1, GROβ/CXCL2, and GROγ/CXCL3, expression of the neutrophil-activating CXC chemokine (ENA-78/CXCL-5), chemokine (C—C motif) ligand 21 (CCL21 or 6Ckine), macrophage inflammatory protein 3 alpha (MIP-3α or CCL20), monokine induced by gamma (MIG or CXCL-9), MIP-1α, chemokine (C—C motif) ligand 5 (CCL-5), also known as RANTES (regulated on activation, normal T cell expressed and secreted), Interleukin-1 alpha (IL-1α), macrophage inflammatory protein-1β (MIP-1β or CCL4), tumor necrosis factor (TNF-α), and monocyte chemotactic protein 2 (MCP-2 or CCL8).

Exemplary anti-inflammatory cytokines include interleukin 8 (IL-8), interleukin 13 (IL-13), interleukin 27 (IL-27), cytotoxic T-lymphocyte-associated protein 4 (CTLA-4), vascular endothelial growth factor D (VEGF-D), interleukin-1 receptor antagonist (IL-IRa), transforming growth factor beta 1 (TGFβ1), interleukin 5 (IL-5), and interleukin 21 (IL-21).

Additional Therapeutic, Prophylactic, and/or Diagnostic Agents

In some embodiments, d-MAPPS compositions (e.g., d-MAPPS solutions that include MSC, MSC-Exos, and/or one or more MSC-sourced growth factors and/or immunoregulatory proteins) are used in combination with one or more additional therapeutic, diagnostic, and/or prophylactic agents to alleviate pain (e.g., pain associated with one or more diseases, including, for instance, THO and/or one or more eye diseases), facilitate healing, and/or to reduce or inhibit scarring. In at least an additional embodiment, the d-MAPPS compositions (e.g., d-MAPPS solutions that include MSC, MSC-Exos, and/or one or more MSC-sourced growth factors and/or immunoregulatory proteins) comprise one or more additional compounds to prevent or treat THO and/or one or more eye diseases (e.g., dry eye disease, dry eye discomfort, tear hyperosmolarity, tear hyperosmolarity-induced changes in patients suffering from dry eye discomfort), and/or to relieve symptoms such as inflammation. Non-limiting examples include antimicrobial agents, analgesics, local anesthetics, anti-inflammatory agents, antioxidants, immunosuppressants, anti-allergenic agents, enzyme cofactors, essential nutrients, and growth factors.

In some cases, one or more additional active agents may be dispersed in, or otherwise associated with particles in, d-MAPPS compositions (e.g., d-MAPPS solutions that include MSC, MSC-Exos, and/or one or more MSC-sourced growth factors and/or immunoregulatory proteins). In certain embodiments, one or more additional active agents may also be dissolved or suspended in the pharmaceutically acceptable carrier.

In at least one embodiment, the active agents include, for instance, small molecules, biomolecule, peptides, sugar, glycoproteins, polysaccharides, lipids, nucleic acids, and/or combinations thereof. Suitable small molecule active agents include, but are not limited to, organic and organometallic compounds. In at least one instance, the aforementioned small molecule active agent has a molecular weight of less than about 2000 g/mol, more preferably less than about 1500 g/mol, and most preferably less than about 1200 g/mol. The small molecule active agent can be a hydrophilic, hydrophobic, or amphiphilic compound. In at least one example, one or more additional agents may be dispersed, dissolved, and/or suspended in one or more d-MAPPS compositions (e.g., d-MAPPS solutions that include MSC, MSC-Exos, and/or one or more MSC-sourced growth factors and/or immunoregulatory proteins).

In some cases, the active agent is a diagnostic agent imaging or otherwise assessing the eye. Exemplary diagnostic agents include paramagnetic molecules, fluorescent compounds, magnetic molecules, and radionuclides, x-ray imaging agents, and contrast media.

When used for the treatment of ocular diseases, d-MAPPS compositions (e.g., d-MAPPS solutions that include MSC, MSC-Exos, and/or one or more MSC-sourced growth factors and/or immunoregulatory proteins) may contain one or more ophthalmic drugs to treat, prevent or diagnose a disease or disorder of the eye, including, but not limited to, THO and/or DED. Non-limiting examples of ophthalmic drugs include anti-glaucoma agents, anti-angiogenesis agents, anti-infective agents, anti-inflammatory agents, an analgesic, a local anesthetic, growth factors, immunosuppressant agents, anti-allergic agents, an anti-oxidant, a cytokine, and combinations thereof.

Volume of administration of one or more d-MAPPS compositions (e.g., d-MAPPS solutions that include MSC, MSC-Exos, and/or one or more MSC-sourced growth factors and/or immunoregulatory proteins) is tissue-specific and dependent on the disease, disorder, and/or condition to be treated. Dosages can be readily determined by those of skill in the art. See, e.g., Ansel et al., Pharmaceutical Dosage Forms and Drug Delivery Systems (6th ed.), Williams and Wilkins (1995). Additionally, one or more d-MAPPS compositions (e.g., d-MAPPS solutions that include MSC, MSC-Exos, and/or one or more MSC-sourced growth factors and/or immunoregulatory proteins) may be administered in conjunction with other types of cells, e.g., other exogenous stem cells, pluripotent cells, somatic cells, and/or combinations thereof. In at least one embodiment, one or more therapeutic, prophylactic, and/or diagnostic agents is administered prior to, in conjunction with, and/or subsequent to treatment with one or more one or more d-MAPPS compositions (e.g., d-MAPPS solutions that include MSC, MSC-Exos, and/or one or more MSC-sourced growth factors and/or immunoregulatory proteins).

In other embodiments, one or more therapeutic active agents such as an anti-glaucoma agent, an anti-angiogenesis agent, an anti-infective agent, an anti-inflammatory agent, an analgesic agent, a local anesthetic, a growth factor, an immunosuppressant agent, an anti-allergic agent, an anti-oxidant, and a cytokine are administered prior to, in conjunction with, subsequent to, or alternation with treatment with one or more d-MAPPS compositions (e.g., d-MAPPS solutions that include MSC, MSC-Exos, and/or one or more MSC-sourced growth factors and/or immunoregulatory proteins).

In at least one embodiment, the aforementioned therapeutic, prophylactic and/or diagnostic agents may be administered in a neutral form, or in the form of a pharmaceutically acceptable salt. In at least one example, it may be desirable to prepare a formulation containing a salt of an agent due to one or more of the salt's advantageous physical properties, such as, for example, enhanced stability, a desirable solubility, and/or a desirable dissolution profile.

In at least one embodiment, pharmaceutically acceptable salts are prepared by reaction of the free acid or base forms of an active agent with a stoichiometric amount of the appropriate base or acid in water or in an organic solvent, or in a mixture of the two; generally, non-aqueous media such as, for example, ether, ethyl acetate, ethanol, isopropanol, or acetonitrile are preferred. Pharmaceutically acceptable salts include salts of an active agent derived from inorganic acids, organic acids, alkali metal salts, and alkaline earth metal salts, as well as salts formed by reaction of the drug with a suitable organic ligand (e.g., quaternary ammonium salts). Lists of suitable salts are found, for example, in Adejare et al., Remington: The Science and Practice of Pharmacy (23rd ed.), Academic Press (2020).

In at least one embodiment, one or more d-MAPPS compositions (e.g., d-MAPPS solutions that include MSC, MSC-Exos, and/or one or more MSC-sourced growth factors and/or immunoregulatory proteins) comprise one or more local anesthetics. Non-limiting examples of such local anesthetics include tetracaine, lidocaine, amethocaine, proparacaine, lignocaine, and bupivacaine. In at least one example, one or more additional agents, such as, e.g., a hyaluronidase enzyme, is also added to the one or more d-MAPPS compositions (e.g., d-MAPPS solutions that include MSC, MSC-Exos, and/or one or more MSC-sourced growth factors and/or immunoregulatory proteins) to accelerate and/or improve dispersal of the local anesthetic. In some cases, the active agent is an anti-allergic agent such as olopatadine and/or epinastine.

### Anti-Glaucoma Agents

In some embodiments, the one or more additional active agents is one or more anti-glaucoma agents. Representative anti-glaucoma agents include prostaglandin analogs (such as travoprost, bimatoprost, and latanoprost), beta-andrenergic receptor antagonists (such as timolol, betaxolol, levobetaxolol, and carteolol), alpha-2 adrenergic receptor agonists (such as brimonidine and apraclonidine), carbonic anhydrase inhibitors (such as brinzolamide, acetazolamine, and dorzolamide), miotics (e.g., parasympathomimetics, such as pilocarpine and ecothiopate), seretonergics muscarinics, dopaminergic agonists, and adrenergic agonists (such as apraclonidine and brimonidine).

### Anti-Angiogenesis Agents

In some embodiments, the one or more additional active agents is one or more anti-angiogenesis agents. Representative anti-angiogenesis agents include, but are not limited to, antibodies to vascular endothelial growth factor (VEGF) such as bevacizumab (AVASTIN®) and rhuFAb V2 (ranibizumab, LUCENTIS®), and other anti-VEGF compounds including aflibercept (EYLEA®); MACUGEN® (pegaptanim sodium, anti-VEGF aptamer or EYEOOI) (Eyetech Pharmaceuticals); pigment epithelium derived factor(s) (PEDF); COX-2 inhibitors such as celecoxib (CELEBREX®) and rofecoxib (VIOXX®); interferon alpha; interleukin-12 (IL-12); thalidomide (THALOMID®) and derivatives thereof such as lenalidomide (REVLIMID®); squalamine; endostatin; angiostatin; ribozyme inhibitors such as ANGIOZYME® (Sirna Therapeutics); multifunctional antiangiogenic agents such as NEOVASTAT® (AE-941) (Aetema Laboratories, Quebec City, Canada); receptor tyrosine kinase (RTK) inhibitors such as sunitinib (SUTENT®); tyrosine kinase inhibitors such as sorafenib (Nexavar®) and erlotinib (Tarceva®); antibodies to the epidermal grown factor receptor such as panitumumab (VECTIBIX®) and cetuximab (ERBITUX®), as well as other anti-angiogenesis agents known in the art.

### Anti-Infective Agents

In at least one embodiment, one or more d-MAPPS compositions (e.g., d-MAPPS solutions that include MSC, MSC-Exos, and/or one or more MSC-sourced growth factors and/or immunoregulatory proteins) are used in combination with one or more antimicrobial agents. An antimicrobial agent, at least in the context of the present disclosure, is a substance that inhibits the growth of microbes including, for instance, bacteria, fungi, viruses, and/or parasites. Accordingly, antimicrobial agents include, for example, antiviral agents, antibacterial agents, antiparasitic agents, and anti-fungal agents. Non-limiting examples of antiviral agents include, e.g., ganciclovir and acyclovir. Non-limiting examples of antibiotic agents include, for example, aminoglycosides (e.g., streptomycin, amikacin, gentamicin, and tobramycin), ansamycins (e.g., geldanamycin and herbimycin), carbacephems, carbapenems, cephalosporins, glycopeptides (e.g., vancomycin, teicoplanin, and telavancin), lincosamides, lipopeptides (e.g., daptomycin, macrolides such as azithromycin, clarithromycin, dirithromycin, and erythromycin), monobactams, nitrofurans, penicillins, polypeptides (e.g., bacitracin, colistin, and polymyxin B), quinolones, sulfonamides, and tetracyclines.

Other exemplary antimicrobial agents include, for instance, iodine, silver compounds, moxifloxacin, ciprofloxacin, levofloxacin, cefazolin, tigecycline, gentamycin, ceftazidime, ofloxacin, gatifloxacin, amphotericin, voriconazole, natamycin.

### Anesthetics

In at least one embodiment, one or more d-MAPPS compositions (e.g., d-MAPPS solutions that include MSC, MSC-Exos, and/or one or more MSC-sourced growth factors and/or immunoregulatory proteins) are administered in combination with one or more local anesthetics. A local anesthetic, at least in the context of the present disclosure, is a substance that causes reversible local anesthesia and has the effect of loss of sensation of pain. Non-limiting examples of local anesthetics include ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, psuedococaine, pyrrocaine, ropivacaine, salicyl alcohol, tetracaine, tolycaine, trimecaine, zolamine, and combinations thereof. In at least another aspect of this embodiment, the one or more d-MAPPS compositions include an anesthetic agent in an amount of, e.g., about 0.1%, about 0.2%, about 0.3%, about 0.4%, about 0.5%, about 0.6%, about 0.7%, about 0.8% about 0.9%, about 1.0%, about 2.0%, about 3.0%, about 4.0%, about 5.0%, about 6.0%, about 7.0%, about 8.0%, about 9.0%, or about 10% by weight of the total composition. The concentration of local anesthetics in the one or more d-MAPPS compositions can be therapeutically effective, meaning that the concentration is adequate to provide a therapeutic benefit without inflicting harm to the patient.

Ophthalmic anesthetics are agents that act locally to block pain signals at the nerve endings in the eyes. Some exemplary ophthalmic anesthetics are lidocaine, proparacaine, and tetracaine.

### Anti-Inflammatory Agents

In at least one embodiment, one or more d-MAPPS compositions (e.g., d-MAPPS solutions that include MSC, MSC-Exos, and/or one or more MSC-sourced growth factors and/or immunoregulatory proteins) are administered in combination with one or more anti-inflammatory agents. Anti-inflammatory agents reduce inflammation and include, for instance, steroidal and non-steroidal drugs. Suitable steroidal active agents include, for example, glucocorticoids, progestins, mineralocorticoids, and corticosteroids. Other non-limiting examples of anti-inflammatory agents include triamcinolone acetonide, fluocinolone acetonide, prednisolone, dexamethasone, loteprednol, fluorometholone, ibuprofen, aspirin, and naproxen. Non-limiting examples of immune-modulating drugs include cyclosporine, tacrolimus, and rapamycin. Non-limiting examples of non-steroidal anti-inflammatory drugs (NSAIDs) include ketorolac, nepafenac, and diclofenac.

In at least one embodiment, anti-inflammatory agents are anti-inflammatory cytokines. Non-limiting examples of such cytokines include IL-10, IL-17, TNF-α, TGF-β, IL-35, and others described herein. Anti-inflammatory cytokines in the context of biomaterial implants and tissue grafts are cytokines that induce an anti-inflammatory immune environment or suppress an inflammatory immune environment. Activation of regulatory T cells, Tregs, is involved in the prevention of rejection, and the induction and maintenance of peripheral tolerance of the allograft. Th17 cells are a subset of T helper cells which is characterized by the production of IL-17. Th17 cells have been suggested to play a role in allograft rejection. In some embodiments, cytokines to be added to the one or more d-MAPPS compositions (e.g., d-MAPPS solutions that include MSC, MSC-Exos, and/or one or more MSC-sourced growth factors and/or immunoregulatory proteins) are those that induce Tregs activation (e.g. IL-25) and suppress Th17 activation (e.g., IL-10) for minimizing rejection.

### Growth Factors

In at least one embodiment, one or more d-MAPPS compositions (e.g., d-MAPPS solutions that include MSC, MSC-Exos, and/or one or more MSC-sourced growth factors and/or immunoregulatory proteins) are administered in combination with one or more growth factors. As mentioned above herein, growth factors are proteins and/or glycoproteins capable of stimulating cellular growth, proliferation, and/or cellular differentiation. Non-limiting examples of growth factors include transforming growth factor beta (TGF-β), transforming growth factor alpha (TGF-a), granulocyte-colony stimulating factor (GCSF), granulocyte-macrophage colony stimulating factor (GM-CSF), nerve growth factor (NGF), neurotrophins, platelet-derived growth factor (PDGF), erythropoietin (EPO), thrombopoietin (TPO), myostatin (GDF8), growth differentiation factor-9 (GDF9), acidic fibroblast growth factor (aFGF or FGF-1), basic fibroblast growth factor (bFGF or FGF-2), epidermal growth factor (EGF), vascular endothelial growth factor (VEGF), and hepatocyte growth factor (HGF).

### Cofactors and Essential Nutrients

In at least one embodiment, one or more d-MAPPS compositions (e.g., d-MAPPS solutions that include MSC, MSC-Exos, and/or one or more MSC-sourced growth factors and/or immunoregulatory proteins) are administered in combination with one or more enzyme cofactors, and/or one or more essential nutrients. Non-limiting examples of such cofactors include vitamin C, biotin, vitamin E, and vitamin K. Non-limiting examples of such essential nutrients include amino acids, fatty acids, etc.

### Cells and Tissues

In at least one embodiment, one or more d-MAPPS compositions (e.g., d-MAPPS solutions that include MSC, MSC-Exos, and/or one or more MSC-sourced growth factors and/or immunoregulatory proteins) comprise at least one eukaryotic cell type, including, for instance, at least one cell type other than one or more types of MSCs. Non-limiting examples of such eukaryotic cell types include non-mesenchymal stem cells, immune cells (e.g., T lymphocytes, B lymphocytes, natural killer cells, macrophages, dendritic cells), and combinations thereof. In at least an additional embodiment, the cells used are cells that dampen one or more inflammation responses (e.g., regulatory T cells). In at least a further embodiment, exosomes are generated ex vivo from one or more types of MSCs.

### Formulations

The d-MAPPS compositions (e.g., d-MAPPS solutions that include MSC, MSC-Exos, and/or one or more MSC-sourced growth factors and/or immunoregulatory proteins) can be administered in concentrated form, diluted with sterile water or buffer, formulated as a gel, ointment, or suspension. It can include additional therapeutic, prophylactic or diagnostic agents, either in the solution, gel, ointment or suspension, or as particles (nanoparticles, liposomes, microparticles) or implants. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions.

### Solutions, Gels, Ointments, and Suspensions

Numerous ophthalmological formulations are known and available. The d-MAPPS solutions can include sterile filtered amniotic fluid, concentrated or diluted with water, buffered saline, or an equivalent, formed into a gel with a polysaccharide such as alginate or hyaluronic acid, polyvinyl pyrrole, or ointment such as petrolatum or mineral oil, or emulsified with lipid or oil. Ophthalmic emulsions are generally dispersions of oily droplets in an aqueous phase. There should be no evidence of breaking or coalescence.

Ophthalmic suspensions generally contain solid particles dispersed in a liquid vehicle; they must be homogeneous when shaken gently and remain sufficiently dispersed to enable the correct dose to be removed from the container. A sediment may occur, but this should disperse readily when the container is shaken, and the size of the dispersed particles should be controlled. The active ingredient and any other suspended material must be reduced to a particle size small enough to prevent irritation and damage to the cornea.

Ophthalmic ointments are generally sterile, homogeneous, semi-solid preparations intended for application to the conjunctiva or the eyelids. They are usually prepared from non-aqueous bases, e.g., soft paraffin (Vaseline), liquid paraffin, and wool fat. They may contain suitable additives, such as antimicrobial agents, antioxidants, and stabilizing agents.

When the solution is dispensed in a multidose container that is to be used over a period of time longer than 24 hours, a preservative must be added to ensure microbiologic safety over the period of use.

Ideally, the pH of ophthalmic drops should be equivalent to that of tear fluid, which is 7.4. However, the decision to add a buffering agent should be based on stability considerations. The pH selected should be the optimum for both stability of the active pharmaceutical ingredient and physiological tolerance. If a buffer system is used, it must not cause precipitation or deterioration of the active ingredient. The influence on the lachrymal flow should also be taken into account.

Although solutions with the same pH as lacrimal fluid (7.4) are ideal, the outer surfaces of the eye tolerate a larger range, 3.5 to 8.5. The normal useful range to prevent corneal damage is 6.5 to 8.5. The final pH of the solution (e.g., one or more d-MAPPS solutions) is often a compromise, because many ophthalmic drugs have limited solubility and stability at the desired pH of 7.4. Buffers or pH adjusting agents or vehicles can be added to adjust and stabilize the pH at a desired level. Ophthalmic solutions are ordinarily buffered at the pH of maximum stability of the drug(s) they contain. The buffers are included to minimize any change in pH during the storage life of the drug; this can result from absorbed carbon dioxide from the air or from hydroxyl ions from a glass container. Changes in pH can affect the solubility and stability of drugs; consequently, it is important to minimize fluctuations in pH. The buffer system should be designed sufficient to maintain the pH throughout the expected shelf-life of the product, but with a low buffer capacity so that when the ophthalmic solution is instilled into the eye, the buffer system of the tears will rapidly bring the pH of the solution back to that of the tears. Low concentrations of buffer salts are used to prepare buffers of low buffer capacity.

The preparation of aqueous ophthalmic drops (which may be one form of the d-MAPPS solutions disclosed herein) requires careful consideration of the need for isotonicity, a certain buffering capacity, the desired pH, the addition of antimicrobial agents and/or antioxidants, the use of viscosity-increasing agents, and the choice of appropriate packaging. Ophthalmic drops are considered isotonic when the tonicity is equal to that of a 0.9% solution of sodium chloride. The eye can usually tolerate solutions equivalent to 0.5-1.8% of sodium chloride (NaCl).

Solutions that are isotonic with tears are preferred. An amount equivalent to 0.9% NaCl is ideal for comfort and should be used when possible. The eye can tolerate tonicities within the equivalent range of 0.6-2% NaCl without discomfort. There are times when hypertonic ophthalmic solutions are necessary therapeutically, or when the addition of an auxiliary agent required for reasons of stability supersedes the need for isotonicity. A hypotonic ophthalmic solution will require the addition of a substance (tonicity adjusting agent) to attain the proper tonicity range.

The most widely used ophthalmic buffer solutions are boric acid vehicle and Sorensen's modified phosphate buffer. The boric acid vehicle is a 1.9% solution of boric acid in purified water or preferably sterile water. It is isotonic with tears. It has a pH of approximately 5 and is useful when extemporaneously compounding ophthalmic solutions of drugs that are most stable at acid pH. This vehicle does not possess large buffer capacity, but it is sufficient to stabilize pH for the short expiratory periods used for compounded solutions, without overwhelming the natural buffers in lacrimal fluid. The second most commonly used buffer solution is the Sorensen's modified phosphate buffer and is used for drugs needing pH values between the range of 6.5-8.0. This buffer uses two stock solutions, one acidic containing NaH₂PO₄, and one basic containing Na₂HPO₄. The formulas for the stock solutions and their respective proportions used to obtain specific pH values are generally known.

In some instances, the d-MAPPS compositions (e.g., d-MAPPS solutions that include MSC, MSC-Exos, and/or one or more MSC-sourced growth factors and/or immunoregulatory proteins) are distributed or packaged in a liquid form. Alternatively, formulations of the d-MAPPS compositions for ocular administration can be packed as a solid, obtained, for example by lyophilisation of a suitable liquid formulation. The solid can be reconstituted with an appropriate carrier or diluent prior to administration.

The d-MAPPS compositions (for instance, in solution, suspension, and/or emulsion form) for ocular administration may be buffered with an effective amount of buffer necessary to maintain a pH suitable for ocular administration. Suitable buffers are well known by those skilled in the art and some examples of useful buffers are acetate, borate, carbonate, citrate, and phosphate buffers.

The d-MAPPS compositions (for instance, in solution, suspension, and/or emulsion form) for ocular administration may also contain one or more tonicity agents to adjust the isotonic range of the formulation. Suitable tonicity agents are well known in the art and some examples include glycerin, mannitol, sorbitol, sodium chloride, and other electrolytes. Solutions, suspensions, or emulsions for ocular administration may also contain one or more preservatives to prevent bacterial contamination of the ophthalmic preparations. Suitable preservatives are known in the art, and include polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

The d-MAPPS compositions (for instance, in solution, suspension, and/or emulsion form) for ocular administration may also contain one or more excipients known art, such as dispersing agents, wetting agents, and suspending agents.

The ophthalmic drug may be present in its neutral form, or in the form of a pharmaceutically acceptable salt. In some cases, it may be desirable to prepare a formulation containing a salt of an active agent due to one or more of the salt's advantageous physical properties, such as enhanced stability or a desirable solubility or dissolution profile.

Generally, pharmaceutically acceptable salts can be prepared by reaction of the free acid or base forms of an active agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an active agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, MD, 2000, p. 704. Examples of ophthalmic drugs sometimes administered in the form of a pharmaceutically acceptable salt include timolol maleate, brimonidine tartrate, and sodium diclofenac.

### Particles and Implants Containing One or More Therapeutic, Prophylactic or Diagnostic Agents Dispersed in a Polymer Matrix

Particles can also be formed containing one or more therapeutic, prophylactic or diagnostic agents dispersed or encapsulated in a polymeric matrix. The matrix can be formed of non-biodegradable or biodegradable matrices, although biodegradable matrices are preferred. The polymer is selected based on the time required for in vivo stability, e.g., that time required for distribution to the site where delivery is desired, and the time desired for delivery.

Representative synthetic polymers include: poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly (ethylene glycol), polyalkylene oxides such as poly(ethylene oxide), polyalkylene terephthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly (butyric acid), poly(valeric acid), and poly(lactide-co-caprolactone), copolymers and blends thereof. As used herein, “derivatives” include polymers having substitutions, additions of chemical groups, for example, alkyl, alkylene, hydroxylations, oxidations, and other modifications routinely made by those skilled in the art.

Examples of preferred biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), blends and copolymers thereof.

Examples of preferred natural polymers include proteins such as albumin and prolamines, for example, zein, and polysaccharides such as alginate, cellulose and polyhydroxyalkanoates, for example, polyhydroxybutyrate.

The in vivo stability of the matrix can be adjusted during the production by using polymers such as polylactide co glycolide copolymerized with polyethylene glycol (PEG). PEG if exposed on the external surface may elongate the time these materials circulate since it is hydrophilic.

Examples of preferred non-biodegradable polymers include ethylene vinyl acetate, poly(meth)acrylic acid, polyamides, copolymers and mixtures thereof.

Particles having an average particle size of between 10 nm and 1000 microns are useful in the compositions described herein. In preferred embodiments, the particles have an average particle size of between 10 nm and 100 microns, more preferably between about 100 nm and about 50 microns, more preferably between about 200 nm and about 50 microns. In certain embodiments, the particles are nanoparticles having a diameter of between 500 and 700 nm. The particles can have any shape but are generally spherical in shape.

Microparticle and nanoparticles can be formed using any suitable method for the formation of polymer micro- or nanoparticles known in the art. The method employed for particle formation will depend on a variety of factors, including the characteristics of the polymers present in the polymer-drug conjugate or polymer matrix, as well as the desired particle size and size distribution. The type of therapeutic, prophylactic or diagnostic agent(s) being incorporated in the particles may also be a factor as some therapeutic, prophylactic or diagnostic agents are unstable in the presence of certain solvents, in certain temperature ranges, and/or in certain pH ranges.

In circumstances where a monodisperse population of particles is desired, the particles may be formed using a method which produces a monodisperse population of nanoparticles. Alternatively, methods producing poly disperse nanoparticle distributions can be used, and the particles can be separated using methods known in the art, such as sieving, following particle formation to provide a population of particles having the desired average particle size and particle size distribution.

Common techniques for preparing microparticles and nanoparticles include, but are not limited to, solvent evaporation, hot melt particle formation, solvent removal, spray drying, phase inversion, coacervation, and low temperature casting. Suitable methods of particle formulation are briefly described below. Pharmaceutically acceptable excipients, including pH modifying agents, disintegrants, preservatives, and antioxidants, can optionally be incorporated into the particles during particle formation.

Implants can be formed from one or more polymers. In preferred embodiments, the implants are intraocular implants. Suitable implants include, but are not limited to, rods, discs, wafers, and the like.

Implants can also be formed from a polymeric matrix having one or more therapeutic, prophylactic or diagnostic agents dispersed or encapsulated therein. The matrix can be formed of any of the nonbiodegradable or biodegradable polymers described above, although biodegradable polymers are preferred. The composition of the polymer matrix is selected based on the time required for in vivo stability, e.g., that time required for distribution to the site where delivery is desired, and the time desired for delivery. Implants can also be formed from blends of polymer-drug conjugates with one or more of the polymers described above herein.

The implants may be of any geometry such as fibers, sheets, films, microspheres, spheres, circular discs, rods, or plaques. Implant size is determined by factors such as toleration for the implant, location of the implant, size limitations in view of the proposed method of implant insertion, ease of handling, etc.

Where sheets or films are employed, the sheets or films will be in the range of at least about 0.5 mm×0.5 mm, usually about 3 to 10 mm×5 to 10 mm with a thickness of about 0.1 to 1.0 mm for ease of handling. Where fibers are employed, the fiber diameter will generally be in the range of about 0.05 to 3 mm and the fiber length will generally be in the range of about 0.5 to 10 mm.

The size and shape of the implant can also be used to control the rate of release, period of treatment, and drug concentration at the site of implantation. Larger implants will deliver a proportionately larger dose, but depending on the surface to mass ratio, may have a slower release rate. The particular size and geometry of the implant are chosen to suit the site of implantation.

Intraocular implants may be spherical or non-spherical in shape. For spherical-shaped implants, the implant may have a largest dimension (e.g., diameter) between about 5 μm and about 2 mm, or between about 10 μm and about 1 mm for administration with a needle, greater than 1 mm, or greater than 2 mm, such as 3 mm or up to 10 mm, for administration by surgical implantation. If the implant is non-spherical, the implant may have the largest dimension or smallest dimension be from about 5 μm and about 2 mm, or between about 10 μm and about 1 mm for administration with a needle, greater than 1 mm, or greater than 2 mm, such as 3 mm or up to 10 mm, for administration by surgical implantation.

The vitreous chamber in humans is able to accommodate relatively large implants of varying geometries, having lengths of, for example, 1 to 10 mm. The implant may be a cylindrical pellet (e.g., rod) with dimensions of about 2 mm×0.75 mm diameter. The implant may be a cylindrical pellet with a length of about 7 mm to about 10 mm, and a diameter of about 0.75 mm to about 1.5 mm. In certain embodiments, the implant is in the form of an extruded filament with a diameter of about 0.5 mm, a length of about 6 mm, and a weight of approximately 1 mg. In some embodiments, the dimension are, or are similar to, implants already approved for intraocular injection via needle: diameter of 460 microns and a length of 6 mm and diameter of 370 microns and length of 3.5 mm.

Intraocular implants may also be designed to be least somewhat flexible so as to facilitate both insertion of the implant in the eye, such as in the vitreous, and subsequent accommodation of the implant. The total weight of the implant is usually about 250 to 5000 μg, more preferably about 500-1000 μg. In certain embodiments, the intraocular implant has a mass of about 500 μg, 750 μg, or 1000 μg.

Implants can be manufactured using any suitable technique known in the art. Examples of suitable techniques for the preparation of implants include solvent evaporation methods, phase separation methods, interfacial methods, molding methods, injection molding methods, extrusion methods, coextrusion methods, carver press method, die cutting methods, heat compression, and combinations thereof. Suitable methods for the manufacture of implants can be selected in view of many factors including the properties of the polymer/polymer segments present in the implant, the properties of the one or more therapeutic, prophylactic or diagnostic agents present in the implant, and the desired shape and size of the implant. Suitable methods for the preparation of implants are described, for example, in U.S. Pat. No. 4,997,652 and U.S. Patent Application Publication No. US 2010/0124565.

In certain cases, extrusion methods may be used to avoid the need for solvents during implant manufacture. When using extrusion methods, the polymer/polymer segments and therapeutic, prophylactic or diagnostic agent are chosen so as to be stable at the temperatures required for manufacturing, usually at least about 85 degrees Celsius. However, depending on the nature of the polymeric components and the one or more therapeutic, prophylactic or diagnostic agents, extrusion methods can employ temperatures of about 25° C. to about 150° C., more preferably about 65° C. to about 130° C.

Implants may be coextruded in order to provide a coating covering all or part of the surface of the implant. Such coatings may be erodible or non-erodible, and may be impermeable, semi-permeable, or permeable to the therapeutic, prophylactic or diagnostic agent, water, or combinations thereof. Such coatings can be used to further control release of the therapeutic, prophylactic or diagnostic agent from the implant.

Compression methods may be used to make the implants. Compression methods frequently yield implants with faster release rates than extrusion methods. Compression methods may employ pressures of about 50-150 pounds per square inch (psi), more preferably about 70-80 psi, even more preferably about 76 psi, and use temperatures of about 0° C. to about 115° C., more preferably about 25° C.

### Methods of Administration

The compositions and methods disclosed herein, such as the d-MAPPS compositions (e.g., d-MAPPS solutions that include MSC, MSC-Exos, and/or one or more MSC-sourced growth factors and/or immunoregulatory proteins), are suitable for treating one or more of the following: THO, DED, eye discomfort, eye pain, eye dryness, excessive tearing, eye injuries, eye infections, eye burns. In some embodiments, the d-MAPPS compositions (e.g., d-MAPPS solutions that include MSC, MSC-Exos, and/or one or more MSC-sourced growth factors and/or immunoregulatory proteins) are used to alleviate pain, facilitate healing, and/or reduce or inhibit scarring.

The compositions and methods disclosed herein, such as the d-MAPPS compositions (e.g., d-MAPPS solutions that include MSC, MSC-Exos, and/or one or more MSC-sourced growth factors and/or immunoregulatory proteins), are also suitable for prophylactic uses. In some embodiments, the d-MAPPS compositions (e.g., d-MAPPS solutions that include MSC, MSC-Exos, and/or one or more MSC-sourced growth factors and/or immunoregulatory proteins) are used to relieve discomfort associated with extended computer use in human subjects.

Examples of eye disorders that may be treated according to the compositions and methods disclosed herein, such as the d-MAPPS compositions (e.g., d-MAPPS solutions that include MSC, MSC-Exos, and/or one or more MSC-sourced growth factors and/or immunoregulatory proteins), include THO, DED, amoebic keratitis, fungal keratitis, bacterial keratitis, viral keratitis, onchorcercal keratitis, bacterial keratoconjunctivitis, viral keratoconjunctivitis, corneal dystrophic diseases, Fuchs' endothelial dystrophy, meibomian gland dysfunction, anterior and posterior blepharitis, conjunctival hyperemia, conjunctival necrosis, cicatrical scaring and fibrosis, punctate epithelial keratopathy, filamentary keratitis, corneal erosions, thinning, ulcerations and perforations, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, anterior uveitis, posterior uveitis (including toxoplasmosis), pan-uveitis, an inflammatory disease of the vitreous or retina, endophthalmitis prophylaxis and treatment, macular edema, macular degeneration, age-related macular degeneration, proliferative and nonproliferative diabetic retinopathy, hypertensive retinopathy, an autoimmune disease of the retina, primary and metastatic intraocular melanoma, other intraocular metastatic tumors, open angle glaucoma, closed angle glaucoma, pigmentary glaucoma and combinations thereof. Other disorders including injury, burn, or abrasion of the cornea, cataracts and age related degeneration of the eye or vision associated therewith.

In some embodiments, the d-MAPPS compositions (e.g., d-MAPPS solutions that include MSC, MSC-Exos, and/or one or more MSC-sourced growth factors and/or immunoregulatory proteins) can be applied to the eye to dissolve cataracts, reducing cataracts about 5%, about 10%, about 20%, about 30%, about 40%, about 50%, about 60%, about 70%, about 80%, about 90%, or more than 90%, in size. In other embodiments, the d-MAPPS compositions (e.g., d-MAPPS solutions that include MSC, MSC-Exos, and/or one or more MSC-sourced growth factors and/or immunoregulatory proteins) dissolve cataracts, eliminating the need for an operation to remove cataracts. In some embodiments, the d-MAPPS compositions (e.g., d-MAPPS solutions that include MSC, MSC-Exos, and/or one or more MSC-sourced growth factors and/or immunoregulatory proteins) are used to assist recovery from a cataract removal procedure.

The d-MAPPS compositions (e.g., d-MAPPS solutions that include MSC, MSC-Exos, and/or one or more MSC-sourced growth factors and/or immunoregulatory proteins) may be administered to animals, especially mammalian animals for treating or alleviating pain, disease, disorder, infection, or injury of the eye. Mammalian subjects, include, but are not limited to, humans, primates such as monkeys and apes, canines such as dogs, felines such as cats, bovines such as cows, equines such as horses, swine such as pigs, and rodents such as mice and rats. In some embodiments, the d-MAPPS compositions (e.g., d-MAPPS solutions that include MSC, MSC-Exos, and/or one or more MSC-sourced growth factors and/or immunoregulatory proteins) are used to relieve/treat THO, DED, dry eye, and/or eye infections, and/or to improve vision or to assist in the recovery from a surgical procedure on the eye in mammals such as dogs, cats, rabbits, and horses.

Case studies have shown an immediate positive disease modification for patients with mild to moderate and severe THO, dry eye syndrome, glaucoma, Sjogren's syndrome, possible Ankylosing spondylitis, and age-related declining vision. Due to the viscosity of at least some of the d-MAPPS compositions (e.g., d-MAPPS solutions that include MSC, MSC-Exos, and/or one or more MSC-sourced growth factors and/or immunoregulatory proteins), drops applied directly onto the eye adhere to the ocular surface longer than common over the counter (“OTC”) artificial tear formulas. The capacity to adhere to the ocular surface is paramount when treating injuries and diseases such as Sjogren's syndrome and chemical burns. Some unexpected results reported in the study were perceptible improvement to clarity of vision which had been diminished in several patients. Relief from varying levels of ocular discomfort or pain was observed. Nine (9) patients were administered Snell Eye Chart exams at the start and completion of the initial 30 day study of the d-MAPPS therapy. Five (5) of the nine demonstrated enriched visual acuity and consistently conveyed improvements in visual clarity, distance and reading ability.

Improvements of one to several lines on the test charts were recorded. Only two patients tested at undetectable improvement levels. Visual acuity appeared to be correlated to the level of corneal integrity of the recipient. This was an unexpected benefit from the d-MAPPS therapy and treatments. Other unexpected benefits were being able to read at night for the first time in years and regaining the visibility required to drive a car. Most participants were able to discontinue or drastically reduce the amount and frequency of using additional applications of artificial tears (“AT”) drops and or alternate curatives. One participant diagnosed with mild dry eye exhibited no signs of the disease at the end of the initial 30 day trial.

### Ocular Burns

In some embodiments, the formulations and methods described herein, such as the d-MAPPS compositions (e.g., d-MAPPS solutions that include MSC, MSC-Exos, and/or one or more MSC-sourced growth factors and/or immunoregulatory proteins), are used for assisting recovery from ocular burns, or from procedures managing ocular burns such as autolimbal or allolimbal transplantation.

Ocular burns such as thermal and chemical burns represent potentially blinding ocular injuries. Thermal burns result from accidents associated with firework explosions, steam, boiling water, or molten metal (commonly aluminum). Chemical burns may be caused by either alkaline or acidic agents. Common alkaline agents include ammonium hydroxide used in fertilizer production, sodium hydroxide (caustic soda) used for cleaning drains and pipes, and calcium hydroxide found in lime plaster and cement.

Alkaline agents are particularly damaging as they have both hydrophilic and lipophilic properties, which allow them to rapidly penetrate cell membranes and enter the anterior chamber. Alkali damage results from interaction of the hydroxyl ions causing saponification of cell membranes and cell death along with disruption of the extracellular matrix. Common acidic agents causing injury include sulphuric acid found in car batteries, sulphurous acid found in some bleaches, and hydrochloric acid used in swimming pools. Acids tend to cause less damage than alkalis as many corneal proteins bind acid and act as a chemical buffer. In addition, coagulated tissue acts as a barrier to further penetration of acid. Acid binds to collagen and causes fibril shrinkage.

Recovery of ocular surface burns depends upon the causative agent and the extent of damage to corneal, limbal, and conjunctival tissues at the time of injury. Damage to intraocular structures influences the final visual outcome. Thus, in some embodiments, the d-MAPPS compositions (e.g., d-MAPPS solutions that include MSC, MSC-Exos, and/or one or more MSC-sourced growth factors and/or immunoregulatory proteins) are used to speed the recovery from an ocular burn.

### Ocular Blast Injuries

Ocular blast injuries can be primary, from the blast wave itself; secondary, from fragments carried by the blast wind; tertiary, due to structural collapse or being thrown against a fixed object; or quaternary, from burns and indirect injuries. In some embodiments, the d-MAPPS compositions (e.g., d-MAPPS solutions that include MSC, MSC-Exos, and/or one or more MSC-sourced growth factors and/or immunoregulatory proteins) are used in the management of injuries inflicted by blasts and explosions for preventative and/or therapeutic purposes.

### Eye Surgery

The d-MAPPS compositions (e.g., d-MAPPS solutions that include MSC, MSC-Exos, and/or one or more MSC-sourced growth factors and/or immunoregulatory proteins) are suitable for use in the management of eye surgeries. Eye surgery, ocular surgery, or ophthalmologic surgery, refers to any surgery that is performed on the eye or its adnexa. Exemplary ocular surgeries include laser eye surgery, cataract removal, glaucoma surgery such as canaloplasty, refractive surgery such as LASIK®, corneal surgery, vitreo-retinal surgery, eye muscle surgery, oculoplastic surgery such as eye lid surgery and orbital surgery, surgery involving the lacrimal apparatus, and eye removal.

In some embodiments, the d-MAPPS compositions (e.g., d-MAPPS solutions that include MSC, MSC-Exos, and/or one or more MSC-sourced growth factors and/or immunoregulatory proteins) are used prior, during or after one or more ocular surgeries. Thus, in some embodiments, the d-MAPPS compositions (e.g., d-MAPPS solutions that include MSC, MSC-Exos, and/or one or more MSC-sourced growth factors and/or immunoregulatory proteins) are used along with one or more systemic drugs. For example, at least some of the d-MAPPS compositions are applied as eye drops whilst the patient is on non-steroidal anti-inflammatory drugs such as ibuprofen.

In some embodiments, the d-MAPPS compositions (e.g., d-MAPPS solutions that include MSC, MSC-Exos, and/or one or more MSC-sourced growth factors and/or immunoregulatory proteins) are used to assist recovery from an ocular surgery. In some embodiments, one or more of such d-MAPPS compositions are used to prevent, reduce, or alleviate one or more symptoms from an ocular surgery. For example, one or more of such d-MAPPS compositions can be used during recovery after a surgical procedure of amniotic membrane graft onto the ocular surface. In some embodiments, one or more of such d-MAPPS compositions are used to prevent one or more potential complications from an ocular surgery such as an infection. In some embodiments, one or more of such d-MAPPS compositions are used to assist local tissue repair, and/or minimize scarring of the surgical site.

### Eye Infections

The formulations are suitable for use in the management of eye infections. Eye infections include infections from bacteria, fungi, and viruses. Eye infections can occur in different parts of the eye and can affect just one eye or both. Exemplary eye infections include conjunctivitis, stye, caratitis, and ocular herpes.

In some embodiments, the d-MAPPS compositions (e.g., d-MAPPS solutions that include MSC, MSC-Exos, and/or one or more MSC-sourced growth factors and/or immunoregulatory proteins) are for prophylactic purposes to prevent an outset of a suspected eye infection. For example, if one person with an eye infection, e.g., conjunctivitis, is identified, anyone who has been recently in contact with that person can use the disclosed formulation for prophylactic purposes. In some embodiments, one or more of such d-MAPPS compositions are used to prevent, reduce, or alleviate one or more symptoms from an eye infection.

### Drug-Induced Eye Conditions

The d-MAPPS compositions (e.g., d-MAPPS solutions that include MSC, MSC-Exos, and/or one or more MSC-sourced growth factors and/or immunoregulatory proteins) are also suitable for use in the management of eye problems that arise as a side effect of using one or more systemic drugs.

Thus, in some embodiments, one or more such d-MAPPS compositions are used prior, during or after taking one or more systemic drugs. Exemplary drugs that can cause ocular side effects include corticosteroids, antihistamines, antipsychotic medications, antimalarials, blood pressure medications, herbal medicines, erectile dysfunction drugs, anticholinergics, immunosuppressants, antibiotics, antiarrhythmic agents, and anti-cancer drugs/treatment. Some specific examples are bisphosphonate, amiodarone, tamsulosin, topiramate, ethambutol, minocycline, cyclosporine and tacrolimus.

Corticosteroids used for many conditions such as asthma, allergies, arthritis and skin conditions can cause swelling in the back of the eye or retina and potentially lead to cataracts. Antihistamines, used for conditions such as allergies, can raise certain patients' risk for glaucoma. Antipsychotic medications, such as THORAZINE® and MELLARIL® can be toxic to the retina. Antimalarials, such as PLAQUENIL® (hydroxychloroquine), used to treat malaria, lupus and rheumatoid arthritis, is a known retinal toxin, and the effects are irreversible. FOSAMAX®, a bisphosphonate that is prescribed for post-menopausal women to prevent calcium bone loss, can cause orbital inflammation, uveitis and scleritis.

Cyclosporine and Tacrolimus, commonly used in patients who have undergone organ or bone marrow transplants, can cause posterior reversible encephalopathy syndrome. These patients will present with bilateral vision loss. Minocycline is a tetracycline derivative and is commonly used to treat acne. Minocycline can cause increased intracranial pressure and papilledema, which can cause permanent vision loss if not reversed. Ethambutol is widely used to treat mycobacterial disease, including tuberculosis. If it is not taken at safe doses, it is an optic nerve toxin. Topiramate (Topamax) is used to treat epilepsy and migraine headaches, and it is used off-label for weight loss. It can cause angle-closure glaucoma soon after starting treatment.

Tamsulosin (Flomax), which is used to treat prostate enlargement and improve urinary flow in men. The well-known syndrome, intraoperative floppy iris syndrome, used to occur only in men who were on medicine to relax their prostate. Women with these drugs can at the time of cataract surgery, make surgical risk much higher. Amiodarone (Cordarone) effectively treats cardiac arrhythmias. It causes the appearance of a whorl in the cornea, which does not usually cause symptoms, although some people can have a little bit of blurred vision.

Anticholinergics, e.g., dicyclomine (BENTYL®), and other drugs with anticholinergic effects, are administered to patients who have stomach conditions that require stomach relaxers and to patients with Parkinson's disease. Young patients taking these drugs will develop difficulty with accommodation. Erectile dysfunction drugs, e.g., sildenafil citrate (VIAGRA®) and tadalafil (CIALIS®) are often prescribed for men with erectile dysfunction. Some of the ocular side effects include blue vision, and ischemic optic neuropathy. Further, blood pressure medications can cause glaucoma.

In some embodiments, the formulations and methods disclosed herein, such as the d-MAPPS compositions (e.g., d-MAPPS solutions that include MSC, MSC-Exos, and/or one or more MSC-sourced growth factors and/or immunoregulatory proteins), are used for treating, alleviating, and/or preventing one or more ocular symptoms that arise as a side effect from taking a systemic drug.

In some embodiments, the formulations and methods disclosed herein, such as the d-MAPPS compositions (e.g., d-MAPPS solutions that include MSC, MSC-Exos, and/or one or more MSC-sourced growth factors and/or immunoregulatory proteins), are used for treating, alleviating, and/or preventing one or more ocular symptoms in patients with THO and/or DED. Such symptoms include, for instance, dry eye discomfort, tear hyperosmolarity, and/or tear hyperosmolarity-induced pathological changes in the eyes of patients suffering from dry eye discomfort. Other exemplary ocular manifestations that can be treated include moderate to severe keratoconjuncitvitis sicca, bilateral marginal keratitis, anterior uveitis, corneal ulceration or neovascularization. Thus, in some embodiments, the d-MAPPS compositions (e.g., d-MAPPS solutions that include MSC, MSC-Exos, and/or one or more MSC-sourced growth factors and/or immunoregulatory proteins) are suitable for treating, alleviating, and/or preventing keratoconjuncitvitis sicca, bilateral marginal keratitis, anterior uveitis, corneal ulceration or neovascularization.

### Dosages and Dosing Regimens

Specific d-MAPPS compositions (e.g., d-MAPPS solutions) that include human amniotic fluid, and methods of use thereof, have been developed for topical application to the eye, for the treatment of ocular diseases and injuries including dry eyes, Sjogren's syndrome, cataracts, burns and injuries to the eye tissues. The method involves the management of a specifically formulated diluted sterile decellularized human amniotic fluid applied directly to the eye(s), preferably as a liquid ocular solution, much like a common liquid eye drops, lubricant or gel. The d-MAPPS compositions delivered to the surface of the eye can alleviate or prevent at least one symptom of a number of ocular injuries and diseases, including in addition to chronic dry eye disease, Sjogren's syndrome, and burns or injuries, corneal neovascular disorders, corneal opacities (including corneal haze), prolonged redness and inflammation of the eye(s).

Such d-MAPPS compositions have been tested and shown to contain over 300 human growth factors, which can stimulate the proliferation of stem cells, thereby accelerating healing and contributing to modifying the advancement of disease. Due to the viscosity of at least one of such d-MAPPS compositions, drops applied directly onto the eye adhere to the ocular surface longer than common OTC artificial tear formulas. The capacity to adhere to the ocular surface is paramount when treating injuries and diseases such as Sjogren's syndrome and chemical burns.

Unlike Human Amniotic Membrane (HAM) treatments, in the preferred embodiment, one or more d-MAPPS compositions are provided as a single daily application provided by a licensed ophthalmic profession for in-home use by patients. Therefore, nonsurgical ophthalmologists and optometrists can dispense and oversee the therapy, giving patients greater choices and access to treatment. In addition, unlike the surgical application of HAM, daily applications of such d-MAPPS compositions deliver a sustainable level of beneficial growth factors. Further, the d-MAPPS compositions require much less manipulation during processing and is sterilized without the harsh terminal irradiation or e-beam required for HAM.

As demonstrated by the applications, the concentration and dosage (number of times per day of amount of formulation for period of time) will vary depending on the condition to be treated, the severity of the condition, and the inclusion of other therapeutic, prophylactic or diagnostic agents. The appropriate amounts are determined on an individual basis, measuring response to treatment over time, as demonstrated in the examples. In most cases, two to three drops of solution will be administered once or twice daily as needed.

The dilution ratio of at least some of these d-MAPPS compositions will be dependent on the severity of the disorder or injury; for example, early to moderate dry eye or chronic redness, surface inflammation and, intraocular inflammation may be best treated with a low concentration, whereas, Sjogren's syndrome, severe dry eye, a corneal neovascular disorder, or corneal opacity may dictate a higher concentration of these d-MAPPS compositions.

In the case of sustained or controlled release formulations, ointments, implants or injections into the eye, the dosages will be modified to deliver a therapeutically equivalent amount.

Embodiments of the present disclosure will be further understood by reference to the following non-limiting examples. The examples showing preparation of human amniotic formulation are from U.S. Patent Publication No. US2015/0025366.

### EXAMPLES

### Example 1: Preparation of d-MAPPS Solutions

### Materials and Methods

A non-limiting example of a d-MAPPS solution is an engineered biological product obtained from MSCs (e.g., placental tissue-derived MSCs (“PL-MSC” or “PL-MSCs”)) and/or the amniotic fluid from healthy human donors. Human placental tissue and amniotic fluid was collected from healthy human donor Caesarean sections, as described above. Amniotic fluid and placental tissue were stored in refrigerated condition at 2.5° C. to 6.5° C. prior to the clarification and filtration process. Regarding amniotic fluid preparation, amniotic fluid was centrifuged at 5,000 to 10,000 rpm for 20 minutes to 1 hour in 50 mL to 250 mL receptacles. The supernatant was collected. When collecting the supernatant, it was important to avoid detaching or aspirating insoluble components. If the supernatant contained residual insoluble components, they were pre-filtered with 5 to 10μ cellulose ester capsule pre-filters without TRITON® surfactant to avoid contamination in the filtration process. The liquid phase was collected and filtered with poly ether sulfone 1.0μ capsule filters and the liquid was collected. The liquid was then filtered with poly ether sulfone 0.25μ capsule filter. The filtrate was transferred to vials and sealed with stoppers aseptically. Four samples from the final filtrate were taken to test whether the sterile filtered human amniotic fluid retained exogenous immune cells of interest. Generally, the concentration of the exogenous immune cells in the sterile filtered amniotic fluid was from about 20 μg/mL to about 2400 μg/mL. The concentrations of all the exogenous immune cells in the four samples were in the range of 20-150 pg/mL.

The d-MAPPS solution may also include MSC-Exos and/or MSC-Exos-derived growth factors and/or cytokines. PL-MSC were grown in complete DMEM. Low passage (<5) PL-MSCs were grown to 60%-80% confluence in multiflasks before isolation. Fresh PL-MSC media were layered and collected after 48 to 72 h (conditioned medium). Exosomes (“Exos”) were isolated by the ultracentrifugation protocol (100,000 g at 4° C. for 70 min). The isolation of exosomes was performed by positive selection using the μMACS™ Separator (Miltenyi Biotec, Bergisch Gladbach, Germany; Cat. No. 130-042-602) and the Exosome Isolation Kit Pan, human (Miltenyi Biotec, Bergisch Gladbach, Germany; Cat. No. 130-110-912), which contained a cocktail of MicroBeads conjugated to the tetraspanin proteins CD9, CD63, and CD81.

The d-MAPPS solution was further prepared by collecting MSCs and/or amniotic fluid. De-cellularization was then performed to remove only cells and particulate matter by a series of centrifugation and filtration steps (e.g., centrifuged at 5,000 to 15,000 rpm for 15 minutes to 1 hour; filtration of supernatant with 5 to 10μ cellulose ester capsule filters). Next, the de-cellularized liquid was incubated and/or stored from 1° C. to 20° C., from 2° C. to 8° C., at 4° C., or at room temperature, for one or more days, weeks, months, or up to a year.

Before d-MAPPS isolation, blood samples of healthy donors were tested by laboratories certified under the Clinical Laboratory Improvement Amendments (CLIA) and found negative using United States (U.S.) Food and Drug Administration (FDA) licensed tests for detection of: Hepatitis B Virus, Hepatitis C Virus, Human Immunodeficiency Virus Types 1/2, and Treponema pallidum. All of the d-MAPPS samples contain one or more types of MSCs, one or more MSC-Exos (e.g., AF-MSC Exosomes), and one or more MSC-derived and/or MSC-Exos-derived growth factors and/or cytokines (e.g., AF-MSC-derived growth factors and/or cytokines), manufactured under current Good Manufacturing Practices (cGMP), regulated and reviewed by the FDA.

As stated above herein, the growth factors derived from MSCs/MSC-Exos may include, for instance, IL-1Ra, sTNFRI, sTNFRII, GRO-γ, fatty acid-binding protein 1 (FABP1), and/or platelet factor 4 (PF4).

As stated above herein, the cytokines derived from MSCs/MSC-Exos may include, for instance, one or more members of the IL-12 cytokine family (e.g., IL-12, IL-23, IL-27, IL-35), one or more CSC chemokines (e.g., CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17), and/or other immunostimulatory molecules known in the art.

### Results

The d-MAPPS solution was provided to patients with THO and/or DED for attenuating pain, dryness, grittiness, scratchiness, soreness, irritation, burning, watering, and/or eye fatigue. The d-MAPPS solution contains growth factors that (1) suppress IL-1β and TNF-α-driven inflammation, (2) prevent the generation of inflammatory Th1 and Th17 cells, (3) support tear stability, and (4) reduce ocular surface epithelial damage. The d-MAPPS solution may further attenuate, prevent, and/or remedy one or more symptoms of DE and/or DED, such as dry eye discomfort, tear hyperosmolarity, tear hyperosmolarity-induced changes in patients suffering from dry eye discomfort. Preferably, the d-MAPPS solution remedies and/or reverses tear hyperosmolarity.

Patients can be treated with the d-MAPPS solution for at least about 1 week, at least about 2 weeks, at least about 3 weeks, at least about 1 month, or at least about 3 months. The d-MAPPS solution can be administered as eye drops 3-4 times per day (e.g., at about 10 μg/drop), every day. Changes in eye condition can be observed by measuring, for instance, ocular surface disease index, conjunctiva redness scores, ocular surface staining, best corrected visual acuity, tear secretion, tear meniscus height, tear film breakup time (TBUT), and/or tear film thickness.

TBUT is preferably less than about 10 seconds or less than about 5 seconds after one or more courses of treatment with the d-MAPPS solution. Improvements in TBUT may be seen in at least some patients after, for instance, 21 days after topical administration of the d-MAPPS solution. Such improvements may indicate improvement of, and/or restoration of, meibomian gland function.

Tear film thickness, which can be used to measure the liquid layer, the lipid layer, and/or a combination of the liquid layer and the lipid layer, can increase as TBUT increases. Tear film thickness can be measured by, for instance, (1) a slit lamp and a video camera, and/or (2) an optical interferometer (e.g., a wavelength-dependent optical interferometer), as described above herein. Specifically, multiple measurements can be taken of an area of a subject's eye using an optical interferometer. The area has a predetermined length and width (e.g., about 20 μm, about 30 μm, about 50 μm, about 100 μm) and may be located in a suitable area of the eye (e.g., the apex of the cornea). Multiple measurements (e.g., at least about 10, at least about 20, at least about 30, at least about 50) can be taken over a window of time (e.g., 20 seconds to 1 minute). Such tear film thickness measurements may be taken both before and after d-MAPPS solution-based treatment. Before treatment, tear film thickness may be, for example, less than about 2 microns, or less than about 1 micron. After treatment, tear film thickness may be, for example, about 5 microns, about 6 microns, about 8 microns, about 10 microns, about 12 microns, or more than about 12 microns.

Overall, significantly improved visual acuity, relieved ocular pain, and complete healing of corneal epithelial defects may be noticed in at least some patients. Further, the d-MAPPS solution can improve the viability of injured corneal epithelial cells and alleviate the symptoms elicited by corneal injury. In addition, the d-MAPPS solution may (e.g., after four weeks of treatment) result in remarkably improved visual acuity and in significantly decreased ocular pain.

### Example 2: Treatment of Dry Eye Disease (DED) Patients with d-MAPPS Solutions

### Materials and Methods

The d-MAPPS solutions used in this Example are bio-engineered biologic products obtained from amniotic fluid derived MSCs (AF-MSCs), previously collected from healthy human donors. Blood samples were given by the donor prior to, or at the time of, collection. These samples were tested by laboratories certified under the Clinical Laboratory Improvement Amendments (CLIA) and found negative using United States (U.S.) Food and Drug Administration (FDA) licensed tests for detection of at minimum: Hepatitis B Virus, Hepatitis C Virus, Human Immunodeficiency Virus Types 1/2, Treponema pallidum. AF samples were obtained with patient consent and stored at 4° C. until processed.

The d-MAPPS solutions are then bio-engineered as an AF-MSC-derived sterile product containing, among other components, AF-MSC-Exos and AF-MSC-derived cytokines and growth factors. The d-MAPPS solutions may contain additional components (e.g., osmoprotectants) as described above herein. The d-MAPPS solutions may also have specific properties (e.g., pH) as described above herein. The d-MAPPS solutions may also include one or more solutions as described specifically in Example 1. The d-MAPPS solutions are manufactured under current Good Manufacturing Practices (cGMP), regulated and reviewed by the FDA. The d-MAPPS solutions are then sterilized to provide for a safe, sterile product that can be introduced as eye drops into the eyes of patients.

Specifically, a total of 131 DED patients were recruited (27 male and 104 female), with a median age of 62 years (ranging from 19 to 85 years of age). Patients received d-MAPPS solutions and were followed up for 12 months. The principles of Good Clinical Practice and the Declaration of Helsinki were adhered to at all times during the study. All patients were under continuous medical supervision by either their ophthalmologist or optometrist.

### Results

Patients' subjective symptoms were graded numerically using VAS (Visual Analogue pain Score). The scale ranges from 0 (defined as the absence of pain) to 10 (defined as maximum pain). Patients were asked to describe their discomfort or pain using VAS.

The Standard Patient Evaluation of Eye Dryness Questionnaire (SPEED) questionnaire was also used to evaluate patients' dry eye-related symptoms. The SPEED questionnaire asks about symptoms including dryness or grittiness or scratchiness, soreness or irritation, burning or watering, and eye fatigue. The frequency that patients experience these symptoms is scored from 1 through 3. A score of 1 equates to a frequency of “sometimes,” a score of 2 equates to a frequency of “often,” and a score of 3 equates to a frequency of “constant.” The severity of the symptoms is scored from 0 through 4. A score of 0 represents “no problems or symptoms,” a score of 1 represents a “tolerable” severity, a score of 2 represents an “uncomfortable” severity, a score of 3 represents a “bothersome” severity, and a score of 4 represents an “intolerable” severity.

All patients treated with the d-MAPPS solutions experienced significantly reduced VAS and SPEED scores after 3 months (p<0.001). Specifically, VAS scores declined from a baseline of between 8-9 to between 3-4 after 3 months. SPEED scores declined from a baseline score of between 15-20 to between 5-10 after 3 months. These results indicate that the d-MAPPS solutions managed to improve patient symptoms including pain, dryness, grittiness, scratchiness, soreness, irritation, burning, watering and eye fatigue.

Importantly, the d-MAPPS solutions continued to have beneficial effects after 3 months and, indeed, during the entire 12-month observational period. Specifically, these beneficial effects appear to be significantly increased during the last 6 months of this 12-month period, with the highest reduction in VAS and SPEED scores observed after 12 months of d-MAPPS solution-based therapy. Indeed, patients' VAS and SPEED scores were lower after 12 months (p<0.001) than after 3 months or 6 months. These results indicate that d-MAPPS solution-based treatment provides long-lasting beneficial effects in alleviating ocular symptoms in DED patients.

### Example 3: Preparation of d-MAPPS Compositions Containing Human Amniotic Fluid

### Materials and Methods

Human amniotic fluid is collected from selected Caesarean sections, which make aspiration of the amniotic fluid in clean condition possible. Then the amniotic fluid is stored in refrigerated condition at 2° C. to 6° C. before the clarification and filtration process. The amniotic fluid is centrifuged at 5,000 to 10,000 rpm for 30 minutes to 1 hour in 50 mL to 250 mL swing out buckets. The supernatant is collected. When collecting the supernatant it is important to avoid detaching or aspirating insoluble components possibly coming from the pellet or from the fatty overlayer. If the supernatant still contains residual insoluble components, it may be pre-filtered with 5 to 10μ cellulose ester capsule pre-filters without TRITON® surfactant to avoid contamination in the filtration process. The liquid phase is collected and filtered with poly ether sulfone 1.0μ capsule filters and the liquid is collected. The liquid is then filtered with poly ether sulfone 0.2μ capsule filter. The filtrate is transferred to vials and sealed with stoppers aseptically. Four samples from the final filtrate are taken to test whether the sterile filtered human amniotic fluid retains growth factors, such as human growth hormone, transforming growth factor beta 1, vascular endothelial growth factor, epidermal growth factor, and transforming growth factor beta 3.

The amniotic fluid from the final filtration is aseptically transferred to syringes or vials, then kept in a deep freezer at about −80° C. to about −20° C. for long term storage. The sterile amniotic fluid is dried in the vial via lyophilisation in a built-in a sterile environment. The lyophilisate derived from the amniotic fluid is reconstituted with sterile water before its injection or topical administration. The lyophilisate can be stored at from +4° C. to about +25° C. (room temperature). All of this operation may be carried out in sterile condition and does not need additional sterilization methods such as a final irradiation.

If needed, the lyophilisate derived from amniotic fluid through lyophilisation may be irradiated by e-beam irradiation or gamma ray irradiation to add another guarantee for the final sterility of the powder. Irradiation of a lyophilisate is much less denaturing for proteins and peptides than irradiating aqueous solutions, because the absence of water considerably reduces the production of reactive superoxide anions and their diffusion during irradiation. Such superoxide anions are the main cause of splitting peptide bonds and chemically modifying amino acids of protein and peptides. After lyophilisation, the amniotic fluid is reconstituted by adding the initial volume of water. After gentle homogenization, the powder is quickly dissolved in about one minute.

### Results

The results show retention of growth factors. The concentration of the growth factors in the sterile filtered amniotic fluid is from about 30 μg/mL to about 2500 μg/mL. Except the vascular endothelial growth factor in sample 2, the concentrations of all the factors in the four samples are in the range of 30-150 pg/mL. Although part of growth differentiation factor 11 is lost in centrifugation and filtration, the final sterile filtered amniotic fluid still retains about 17% to 29% of growth differentiation factor from the raw human amniotic fluid.

The reconstituted amniotic liquid is transparent and may be used for wound healing, cosmetic, orthopedic, or ophthalmic applications, particularly for the treatment of dry eyes.

### Example 4: Treatment of Dry Eye Patients with Amniotic Fluid Solution

### Materials and Methods

d-MAPPS composition was prepared as described in Example 3. d-MAPPS compositions were distributed to select patients suffering from the discomfort and pain often accompanied with dry eyes.

The study was designed for ten patients. Three (3) patients entered too late to effectively chart their results. Ultimately, nine (9) patients were officially enrolled in the study.

Study patients were given a 30 day sample of the d-MAPPS composition and instructed to add the therapy of 1-2 drops of the d-MAPPS composition into both eyes twice daily (a.m. and p.m.), to their current prescribed treatments.

The study included the following visual conditions:
Glaucoma;
Chronic Dry Eye;
Moderate Dry Eye;
Mild Dry Eye;
Sjogren's Disease;
Declining sight; and
Ankylosing spondylitis (possible).

The following observations were tracked and recorded:
OSDI Scores;
Visual Acuity;
Redness;
Staining degree;
Tear Break-up Times;
Appearance;
Artificial Tears frequency of use; and
Patient comments.

The Ocular Surface Disease Index (OSDI) was used to determine the base degree of Dry Eye being experienced by the participants.

### Results

The OSDI scores showed consistent improvement with the addition of the d-MAPPS composition to the daily treatment plans. For example, one patient's base score was 47.7. After 2 weeks of treatment the score was reduced to 35, after 3 weeks; 27. This was the general trend with all participants in the study.

Nine (9) participants demonstrated improvement in their visual acuity and consistently demonstrated improvement in distance, visual clarity and reading ability. Improvements of one to several lines on the Snellen Eye Chart were also recorded. Visual acuity seemed to be correlated to corneal integrity levels.

Accordingly, the d-MAPPS composition tested appeared to have a beneficial impact on improving the corneal epithelial integrity which is important for visual acuity. A common complaint associated with Dry Eye is visual fluctuations. Irregularities in the corneal surface is the most accepted explanation for this phenomena and the d-MAPPS composition demonstrated positive assistance for this particular issue.

Redness of the eye is often associated with severe dry eyes. 8 of the 9 participants in this study were classed with severe dry eye and noted improvement in their level of injection.

Staining levels as rated by the Oxford Method, showed improvement in all participants. One participant listed as moderated dry eye, showed no signs of dry eye after the d-MAPPS composition therapy. Overall, reduction as opposed to elimination in staining would be most accurate in describing the universal results. The d-MAPPS composition demonstrated therapeutic benefit for corneal staining with the unexpected decline in the associated use of artificial tear solution for the participants.

Tear Break Up Times (TBUT) are difficult to measure in the limited time of the study. The return of goblet cells to normal levels required extended management of the patient's disease. However, the d-MAPPS composition that persists on the ocular surface for 90 seconds is likely to have a major contributory effect on the hypermolarity level.

Participants in this study, presented with signs of discomfort, high blinking frequency, squinting and other subnormal appearances to their eyes. Within 2 weeks of initiating therapy with the d-MAPPS composition, 8 of 9 participants had demonstrable improvement in their abnormalities. A surprising benefit of the d-MAPPS composition was the expedience in their desire to reduce the use and frequency of an artificial tears solution. One participate classed as Moderate Dry Eye, quit using her artificial tears 3 weeks into the therapy. Others expressed a desire to reduce or eliminate their use of regular artificial tear solutions as well.

Dry Eye Disease continues to be a condition that has no existing cure but must be managed to provide health, well-being and relief to its victims. There has been a widening gap in the therapeutic treatment options for severe dry eye, particularly for the autoimmune aqueous deficient patient. The d-MAPPS composition demonstrated benefits which could augment or possibly replace current forms of dry eye therapy for these patients, as well as milder forms of the disease.

### Example 5: Comparative Study on the Treatment of Corneal Inflammation

### Materials and Methods

The d-MAPPS composition of Example 3 was dissolved in 1 mL of sterile water to reconstitute 1 mL of the initial sterile filtered amniotic fluid. Two drops were applied on each eye of ten patients suffering from the dry eye syndrome. This treatment was repeated twice per day for ten days. Two other control groups of 10 patients similarly received either their own serum or a serum prepared from cord blood as described by Kyung-Chul Yoon (Umbilical cord and its blood: A perspective on its current and potential use in Ophthalmology, in “Regenerative Medicine Using Pregnancy-Specific Biological Substances,” Springer ed. 2011).

### Results

8 to 10 patients out of 10 in each group declared that they had experienced a significant benefit. For all patients, this clinical improvement was correlated with a partial or complete decrease of their initial corneal inflammation. Nine patient had “severe” dry eye, one was “moderate.” The latter is a 70 year old woman, still working, at a computer all day, who has had dry eye for many years, as well as a long history of allergies, asthma, uses an inhaler as well as an antihistamine, and has had the red eyes associated with this problem. The treatment eliminated the redness and significantly reduced light sensitivity and need for artificial tears. The treatment also restored her ability to read books.

### Example 6: Treatment of Glaucoma

A 90 year-old female patient with glaucoma had been on topical medications for glaucoma. Prior to the study, she had declining vision, persistent central corneal staining and suffered from general dry eye for many years due to incomplete blinking patterns and a tendency to sleep with her eyes partially open. She completed a six-week therapy of d-MAPPS composition drops (e.g., one or more d-MAPPS solutions as described above herein) (twice a day) along with artificial tears. Artificial tears were used eight times a day with a reduced frequency over the period of 6 weeks.

Staining patterns clearly improved after the six-week application but did not resolve completely. Her visual acuity and reading ability improved and as well as her comfort level.

| | | | OD | | OS | |
|---|---|---|---|---|---|---|
| | | | VA; TBUT (sec); | | VA; TBUT (sec); | |
| Patient | OSDI | | Schirmer | | Schirmer | |
| #1 | Before | After | Before | After | Before | After |
| G.G. | 52.5 | 21.87 | 20/40; | 20/30; | 20/40; | 20/30 − |
| | | | 4; — | 2; 12 | 2; — | 2; 2; 12 |

In the above table and in other tables in the Examples, OSDI refers to the dry eye ocular surface disease index; OD refers to oculus dexter, the right eye; OS refers to oculus sinister, the left eye; VA refers to visual acuity; and TBUT refers to the tear break up time, the time it takes for the tear film to start evaporating. The longer it takes for the tear film to break up, the more stable the tear. Schirmer's test determines whether the eye produces enough tears to keep it moist.

### Example 7: Treatment of Age-Related Eye Degeneration

An 81 year-old female patient presented with a poor physical appearance due to closed eyes, minimal eye contact and generally downward posture. Prior to the study, she constantly complained about eye discomfort and sensitivity to light. She had uncontrolled dry eye for the past 10 years and had tried multiple types of therapy with no obvious improvement.

Debris and scurf were observed on her eyelids and eyelashes. She had dementia and was under assisted living condition. She completed a four-week therapy of amniotic fluid drops (twice a day) along with artificial tears. Artificial tears were applied many times a day, depending on the aid's availability with a reduced frequency over the period of four weeks.

Additional methods were used along with the eye drops, including hot lid soaks, gentle cleaning and use of artificial tears of a preservative-free variety.

After the therapy, both the patient and her caregivers noticed significant improvement in the comfort level and life style.

| | | | OD | | OS | |
|---|---|---|---|---|---|---|
| | | | VA; TBUT (sec); | | VA; TBUT (sec); | |
| Patient | OSDI | | Schirmer | | Schirmer | |
| #2 | Before | After | Before | After | Before | After |
| E.G. | 58.3 | 33 | 20/40; 4; | 20/30; 4; 3 | 20/40; 4; | 20/30; 4; 3 |
| | | | 6 (unsure) | | 6 (unsure) | |

### Example 8: Treatment of Moderate Dry Eye

A 71 year-old female patient with moderate dry eye resulted from sustained work at a computer for the past 20 years. She had not attained a very comfortable level with the traditional dry eye treatment and had been seeking better therapy. She had a history of allergies. She completed a four-week therapy of amniotic fluid drops (twice a day) along with artificial tears. She used artificial tears more than eight times a day initially with a gradual declining frequency over time.

After the therapy, she observed great improvement in her eye condition. She reached homeostasis and her eyes were comfortable throughout the day. She was almost free of dry eye towards the end of her therapy period although she felt further improvement if the drops were used.

| | | | OD | | OS | |
|---|---|---|---|---|---|---|
| | | | VA; TBUT (sec); | | VA; TBUT (sec); | |
| Patient | OSDI | | Schirmer | | Schirmer | |
| #3 | Before | After | Before | After | Before | After |
| L.J. | 37.5 | 10.41 | 20/20; 5; | 20/20; 8; | 20/20; 5; | 20/20; 8; |
| | | | 10-11 | 16 | 11 | 16 |

### Example 9: Treatment of Sjorgen's Syndrome

A 77 year-old female patient presented with Sjogren's syndrome and dry eye for 20 years. She had an overall good appearance, mild injection and an anterior blepharitis grade-1 mild stye on superior left lid which was resolving. Prior to the study, she had declining vision and uncomfortable dry eyes.

She completed a five-week therapy of d-MAPPS composition drops (e.g., one or more d-MAPPS solutions as described above herein) along with artificial tears. The drops were applied two times a day for the first three weeks, then three times a day for the rest of the period. Artificial tears were applied inconsistently throughout.

This patient improved in a number of areas: comfort, appearance, light-sensitivity, ability to read, general seeing ability, clinical staining signs, and had a number of positive comments to say about the outcome. The patient noted improvement very early in the therapy, and cumulative improvement was appreciated by the patient in the above listed ways as the therapy progressed.

| | | | OD | | OS | |
|---|---|---|---|---|---|---|
| | | | VA; TBUT (sec); | | VA; TBUT (sec); | |
| Patient | OSDI | | Schirmer | | Schirmer | |
| #4 | Before | After | Before | After | Before | After |
| E.L. | 70.8 | 31.25 | 20/40; | 20/40 + 2; | 20/50; | 20/30; |
| | | | 2; 2 | 4; 2 | 2; 2 | 3; 1+ |

### Example 10: Treatment of Dry Eyes

A 64 year-old female patient with dry eyes as a result of her hysterectomy at the age of 38 was treated. She had been diabetic for the past 25 years and had been using metformin. She also had rheumatoid arthritis.

Prior to the study, she was less than comfortable in appearance and semi-squinting constantly. She also had complaints of scratchy, sore and burning eyes.

She completed a four-week therapy of d-MAPPS composition drops (e.g., one or more d-MAPPS solutions as described above herein) (twice a day) along with artificial tears.

The use of artificial tears declined over time. She had a much improved vision, sunlight sensitivity, comfort levels and appearance after therapy.

| | | | OD | | OS | |
|---|---|---|---|---|---|---|
| | | | VA; TBUT (sec); | | VA; TBUT (sec); | |
| Patient | OSDI | | Schirmer | | Schirmer | |
| #5 | Before | After | Before | After | Before | After |
| L.Z. | 77 | 8.3 | 20/40−; | 20/40; | 20/50; | 20/40−; |
| | | | 3; 3 | 4; 2 | 3; 1 | 4; 2 |

### Example 11: Treatment of Dry Eyes and Mouth

A 40 year-old female patient diagnosed with Sjogren's syndrome in 2003 was treated. She noted dry mouth and subsequently dry eye problems. She was overall in good health with no joint pain or swelling, although her appearance was uncomfortable with constant squinting and blinking. She had severe light sensitivity and burning sensation in her eyes. She preferred to keep her eyes closed if possible.

She completed a four-week therapy of d-MAPPS composition drops (e.g., one or more d-MAPPS solutions as described above herein) (twice a day) along with artificial tears, which were applied eight times a day for four weeks.

After the therapy, the patient reported improvement in redness and light sensitivity, comfort level and abilities. Clinical examination identified a significant staining present, suggesting analgesic benefits to the d-MAPPS drops that suppressed the clinical evidence of corneal staining.

| | | | OD | | OS | |
|---|---|---|---|---|---|---|
| | | | VA; TBUT (sec); | | VA; TBUT (sec); | |
| Patient | OSDI | | Schirmer | | Schirmer | |
| #6 | Before | After | Before | After | Before | After |
| B.M. | 47.7 | 12.5 | 20/50; | 20/40−; | 20/40; | 20/40; |
| | | | 1; 1 | 2; 1-2 | 1; 1 | 2; 1-2 |

### Example 12: Treatment of Dry Eye and Light Sensitivity

A 59 year-old female patient with questionable health conditions was treated. She had a recent weight loss with unexplained reasons, chronic back pain from previous injury as well as rheumatoid arthritis.

Prior to the study, she had dry eye for more than 10 years along with a severe light sensitivity. She also had mild redness in her eyes, swollen superior lid appearance and clumping of eyelashes due to anterior blepharitis. She complained of severe discomfort in her eyes and had no relief from traditional artificial tears. The chief source of her problem was the meibomian gland dysfunction of the “obstructive” type that rendered her inadequate protection of tear evaporation.

She completed a four-week therapy of d-MAPPS composition drops (e.g., one or more d-MAPPS solutions as described above herein) (twice a day) combined with artificial tears. Artificial tears was used 10 times a day but was later reduced to three times a day during the therapy period.

An improvement in appearance and comfort levels was observed upon the completion of the therapy.

| | | | OD | | OS | |
|---|---|---|---|---|---|---|
| | | | VA; TBUT (sec); | | VA; TBUT (sec); | |
| Patient | OSDI | | Schirmer | | Schirmer | |
| #7 | Before | After | Before | After | Before | After |
| B.D. | 95.8 | 54 | 20/40; | 20/40; | 20/40; | 20/30; |
| | | | 5; 4 | 8; 10 | immediately; | 8; 10 |
| | | | | | 5 | |

### Example 13: Treatment of Sjorgen's Syndrome

A 74 year old female patient with Sjogren's syndrome and a severe dry eye condition was treated. She had been forced to compromise some areas in her life such as driving, reading, etc.

After a five-week therapy of d-MAPPS composition drops (e.g., one or more d-MAPPS solutions as described above herein) (twice a day) combined with artificial tears (six to eight times a day), she commented that she was able to drive and that her light sensitivity improved after four and a half weeks after therapy. Further, after five weeks of therapy, she reported that she had started reading again after years of inability to do so.

| | | | OD | | OS | |
|---|---|---|---|---|---|---|
| | | | VA; TBUT (sec); | | VA; TBUT (sec); | |
| Patient | OSDI | | Schirmer | | Schirmer | |
| #8 | Before | After | Before | After | Before | After |
| J.P. | 58.3 | 20.8 | 20/30 | 20/25; | 20/20 − 2 | 20/20; |
| | | | (4 days into | 4-5; 1-2 | (4 days into | 4-5; 1-2 |
| | | | therapy); 4; | | therapy); 4; | |
| | | | 1 | | 1 | |

### Example 14: Treatment of Glaucoma

An 80 year-old female patient with glaucoma for 10 years, experiencing loss of vision and dry eye, was treated. After a five and a half-week therapy of d-MAPPS composition drops (e.g., one or more d-MAPPS solutions as described above herein) (twice a day) combined with artificial tears (six times a day), her reading ability, eye staining, dry eye symptoms and standard examination scores have improved.

She had been unable to read prior to therapy, and was back to reading after therapy. She had significant central and inferior corneal staining in punctate and patches prior to therapy, and the patches were all cleared with only less serious punctate fine staining after therapy. She had superficial cornea edema appearing three weeks after therapy, which vanished with a mild hypertonic solution. For alleviating edema, the topical glaucoma medication could be removed and changed to oral acetazolamide in the future.

| | | | OD | | OS | |
|---|---|---|---|---|---|---|
| | | | VA; TBUT (sec); | | VA; TBUT (sec); | |
| Patient | OSDI | | Schirmer | | Schirmer | |
| #9 | Before | After | Before | After | Before | After |
| E.D. | 54 | 22.7 | 20/60; 4; 9 | 20/50+; | 20/60 + 3; | 20/25; 6; 12 |
| | | | | 6; 10 | 4; 6 | |

### Example 15: Management and Treatment of GVHD and oGVHD

Approximately 23,000 hematopoietic stem cell transplants (HSCT) are performed annually in the U.S. and it is estimated that chronic graft versus host disease (cGVHD) occurs in 40-70% of the adult allogeneic HSCT. The risk of cGVHD is significantly lower in the pediatric population (22-29%) than in adults. The incidence of ophthalmologic manifestations of disease is very high, reported to occur in 60-90% of patients with diagnosed cGVHD. The most common manifestation of ocular GVHD (oGVHD) is keratoconjunctivitis sicca (KCS), or dry eye, which occurs in 40-75% of all patients with ocular signs and typically develops within 6-9 months post-HSCT. Ocular GVHD (oGVHD) is characterized by a T cell-mediated immune response that leads to immune cell infiltration and inflammation of ocular structures, including the lacrimal glands, eyelids, cornea and conjunctiva.

A 21 year old male underwent a bone marrow transplant following chemotherapy for Acute Myelogenous Leukemia (AML). He developed severe graft versus host disease that involved his skin, oral mucosa, eyes, and liver. He had bilateral blepharconjunctitivitis with severe dry eye and corneal epithelial changes. His Schirmer testing was 0 in both eyes. He was treated with restasis, artificial tears and periodic steroid eye drops. For previous non-healing corneal ulcers he was also treated with contact lens impregnated with amniotic membrane (Prokera) in both eyes. Because of persistent symptoms and superficial punctate keratitis, he was treated with the d-MAPPS composition eye drops (e.g., one or more d-MAPPS solutions as described above herein) 4 times a day. Following 4 weeks of treatment, the patient noted symptomatic improvement with reduced need for artificial tears and improved clarity of vision. His punctate keratitis improved in both eyes and he is continuing the eye drops. His Schirmer test remained at 0, but that is unlikely to improve in view of his severe GVHD.

Ocular GVHD involves both cell-mediated and humoral immunity that leads to infiltration and inflammation of the lacrimal gland, conjunctiva and ocular surface. The d-MAPPS composition eye drops is a combination eye drop that includes, for instance, cytokines, chemokines and growth factors. Due to the immune nature of ocular GVHD, there is potential for such eye drops to be effective in managing this condition. Larger prospective trials will be useful for investigating the utility of d-MAPPS composition eye drops in the management and treatment of ocular GVHD.

### Overall Summary of the Studies

The d-MAPPS compositions (e.g., d-MAPPS solutions that include MSC, MSC-Exos, and/or one or more MSC-sourced growth factors and/or immunoregulatory proteins), including those described above herein administered in drop form, provide definite and real improvement for THO, dry eye disease, dry eye discomfort, tear hyperosmolarity, and/or tear hyperosmolarity-induced changes in patients suffering from dry eye discomfort. Artificial tears have been the mainstay of dry eye therapy and patients would report that these artificial tears are of no help to their condition, while most clinicians feel they offer no therapeutic benefit. The d-MAPPS compositions feature immediate benefits, e.g., within four days of use, and cumulative improvement as therapy progresses. Patients quickly begin to make lifestyle changes by venturing out more, are not as hindered, note improvements in performance and sustainability during tasks such as using a computer or the ability to stay up later in the evening. Patients' attitudes improve and expectations rise as they sense greater comfort and greater freedom in life, and people are pleased now and at a point of homeostasis. Cosmetic enhancements are noted with all patients due to less injection of bulbar and palpebral conjunctiva. Improvements are noted among a difficult subset of people knows as severe dry eye patients.

Severe dry eye patients often present with compromised appearances due extreme discomfort. Indications of this are habitual squinting, gaze in downward position versus straight ahead, listening to conversation with eyes closed instead of eyes open with good eye contact, high blinking frequency, etc.

A noticeable change in the appearance was apparent in patients in these studies by the end of two weeks of therapy. Other people would comment to these patients that their eyes were looking better. Most patients expressed improvements and increased comfort with therapy. Most patients expressed satisfaction and interest in continuing on the therapy. The majority of the 9 patients studied showed improvements in light sensitivity. One patient reported after two weeks of therapy being able to return to driving after years of avoiding it due to eye discomfort from dryness, sunlight, etc.

The dry eye ocular surface disease index (OSDI) showed a general trend of improvement in OSDI scores as therapy continued.

Frequency of artificial tear use among patients showed a general trend that patients will use less artificial tears after initiating this therapy. This was a surprise early in therapy, often volunteered without prompting. Despite the patients “feeling” like they do not need their previous artificial tears as much as prior to amniotic eye drop therapy, there is objective evidence the patient may benefit from the use more than they are aware. The advantages some artificial tears are meant to provide seem to still benefit the patient, even when the patients are experiencing a new level of soothing and comfort from the use of the d-MAPPS compositions. Supplemental therapy with artificial tears for the moderate dry eye patient, who had no objective clinical evidence of dry eye remaining after three weeks of therapy, showed further improvement in comfort when artificial tears were applied. This observation verifies the hypothesis of what may or may not be accomplished in dry eye therapy. For instance, the forces of evaporation still present challenges to the ocular surface, which are aided by this type of therapy control and management.

Improved reading performance was noted in the majority of the patients, while the other patients had early cataracts developed prior to therapy. Improvements in visual acuity (VA) were noted in the majority of the patients with at least one line on the Snellen chart and in others, two or more. Visual acuity improvements seem closely correlated to corneal integrity levels. When central corneal integrity is compromised as evidenced by corneal staining, visual acuity levels are also compromised. As corneal integrity improves with good therapy, visual acuity also improves as indicated. The d-MAPPS compositions used help heal the corneal surface integrity issues, but are not expected to rehydrate these tissues, and traditional methods of dry eye care may still be advantageous to treat this aspect of dry eye disease. All patients demonstrated improvements in palpebral and bulbar injection levels in essentially all patients within the study.

These and other objectives and features of the invention are apparent in the disclosure, which includes the above and ongoing written specification.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated.

The invention is not limited to the particular embodiments illustrated in the drawings and described above in detail. Those skilled in the art will recognize that other arrangements could be devised. The invention encompasses every possible combination of the various features of each embodiment disclosed. One or more of the elements described herein with respect to various embodiments can be implemented in a more separated or integrated manner than explicitly described, or even removed or rendered as inoperable in certain cases, as is useful in accordance with a particular application. While the invention has been described with reference to specific illustrative embodiments, modifications and variations of the invention may be constructed without departing from the spirit and scope of the invention as set forth in the following claims.

What is claimed is:

1. A method of preventing and/or treating tear hyperosmolarity, the method comprising:
administering to the eye of a subject an effective amount of a topical solution, the topical solution comprising:
one or more types of mesenchymal stem cells (MSC), one or more types of MSC-derived exosomes, one or more MSC-sourced growth factors and/or immunoregulatory proteins, and/or sterile non-heated de-cellularized human amniotic fluid (D-HAF), thereby treating, alleviating, and/or preventing tear hyperosmolarity.

2. The method of claim 1, wherein the administering results in a reduction of activation of transient receptor potential vanilloid 1 (TRPV1) signaling pathway in one or more corneal epithelial cells of the subject.

3. The method of claim 2, wherein the administering results in a reduction of activation of nuclear factor kappa B (NF-κB).

4. The method of claim 3, wherein the administering results in a reduction of expression of one or more inflammatory cytokines, wherein the one or more inflammatory cytokines are selected from the group consisting of: interleukin (IL)-1β, tumor necrosis factor alpha (TNF-α), interferon (IFN)-γ, IL-17, and combinations thereof.

5. The method of claim 1, wherein the administering results in a reduction of one or more selectins on one or more membranes of one or more endothelial cells in a subject, and wherein the one or more selectins are selected from the group consisting of: E selectins, P selectins, and combinations thereof.

6. The method of claim 1, wherein the administering results in a reduction of expression of one or more matrix metalloproteinases.

7. The method of claim 1, wherein the administering results in a reduction of release of one or more neuropeptides, wherein the one or more neuropeptides are selected from the group consisting of: substance P, calcitonin gene-related peptide (CGRP), and combinations thereof.

8. The method of claim 1, wherein the administering results in a reduction of unfolded protein response (UPR) activity in one or more eye cells of the subject.

9. The method of claim 1, wherein the administering results in a reduction of activity of DNA-damage-inducible transcript 3 protein (DDIT3).

10. The method of claim 1, wherein the administering results in a reduction of generation of one or more pro-inflammatory immune cells in the subject.

11. The method of claim 1, wherein the administering results in a reduction of one or more apoptotic pathways in one or more corneal epithelial cells of the subject.

12. The method of claim 1, wherein the administering results in a reduction in neutrophil extracellular trap (NET) formation and/or NETosis.

13. The method of claim 1, wherein the administering results in a reduction of stimulation of natural killer (NK) and natural killer T (NKT) cells.

14. The method of claim 1, further comprising:
administering to the subject one or more additional agents in combination with the topical solution, wherein the one or more additional agents are selected from the group consisting of: an adjuvant, an antigen, an excipient, a vaccine, an allergen, an antibiotic, a gene therapy vector, a kinase inhibitor, a co-stimulatory molecule, a Toll-like receptor (TLR) agonist, a TLR antagonist, a therapeutic agent, a prophylactic agent, a diagnostic agent, an antimicrobial agent, an analgesic, a local anesthetic, an anti-inflammatory agent, an anti-oxidant agent, an immunosuppressant agent, an anti-allergenic agent, an enzyme cofactor, an essential nutrient, a growth factor, and combinations thereof.

15. The method of claim 1, wherein the administering further comprises:
administering, with the topical solution, a pharmaceutically acceptable carrier.

16. The method of claim 1, wherein the topical solution is administered prior to, in conjunction with, subsequent to, or alternating with, one or more therapeutic, prophylactic, and/or diagnostic agents.

17. The method of claim 16, wherein the one or more therapeutic, prophylactic, and/or diagnostic agents is selected from the group consisting of: an anti-glaucoma agent, an anti-angiogenesis agent, an anti-infective agent, an anti-inflammatory agent, an analgesic agent, a local anesthetic, a growth factor, an immunosuppressive agent, an immunomodulatory agent, an anti-allergic agent, an anti-oxidant, a cytokine, and combinations thereof.

18. A topical solution comprising:
one or more types of mesenchymal stem cells (MSC), one or more types of MSC-derived exosomes, one or more MSC-sourced growth factors and/or immunoregulatory proteins, and/or a sterile de-cellularized filtered human amniotic fluid (D-HAF); and
one or more pharmaceutically acceptable excipients,
wherein the topical solution is hypoosmotic relative to an osmolarity of an eye in a subject with tear hyperosmolarity.

19. A kit comprising:
a container containing one or more single, sterile unit doses of the topical solution of claim 18.

20. The kit of claim 19, wherein the one or more single, sterile unit doses is about 0.1 cubic centimeters (cc) to about 10.0 cc, and wherein the one or more single, sterile unit doses is in the form of a solution or equivalent in the form of a lyophilized powder.
